(19) United States
(12) Patent Application Publication
(10) Pub. No.: US 2023/0181268 A1
(43) Pub. Date: Jun. 15, 2023

(54) GUIDED COORDINATED BED MOTION FOR INTRA-OPERATIVE PATIENT POSITIONING IN ROBOTIC SURGERY

(71) Applicant: Auris Health, Inc., Redwood City, CA (US)

(72) Inventors: Yanan HUANG, Sunnyvale, CA (US); Yan WANG, Union City, CA (US); Jason W. CURRIE, Redwood City, CA (US); Sean Patrick KELLY, San Francisco, CA (US); Ryan J. MURPHY, Mountain View, CA (US); Alexander Tarek HASSAN, San Francisco, CA (US); Kai QIAN, Foster City, CA (US); Ying MAO, San Mateo, CA (US)

(21) Appl. No.: 18/106,881

(22) Filed: Feb. 7, 2023

Related U.S. Application Data
(63) Continuation of application No. PCT/IB2021/058610, filed on Sep. 21, 2021.
(60) Provisional application No. 63/086,038, filed on Sep. 30, 2020.

(51) Int. Cl. A61B 34/30; A61B 34/20

(52) U.S. Cl. CPC A61B 34/30; A61B 34/20; A61B 2034/2051; A61B 2034/2059

(57) ABSTRACT

Certain aspects relate to systems and techniques for a patient platform system that includes a table and one or more kinematic chains that are coupled to the table. The table includes a rigid base and a table top that is movable relative to the rigid base. One or more processors initiate first movement of the table top relative to the rigid base in accordance with a user request, and move the one or more kinematic chains relative to the rigid base in coordination with the first movement of the table top such that one or more preset conditions are maintained during the first movement of the table top.

### RELATED APPLICATIONS

This application is a continuation of PCT Patent Application No. PCT/IB2021/058610, filed Sep. 21, 2021, entitled “Guided Coordinated Bed Motion For Intra-Operative Patient Positioning In Robotic Surgery,” which claims priority to U.S. Provisional Patent Application No. 63/086,038, filed Sep. 30, 2020, entitled “Guided Coordinated Bed Motion For Intra-Operative Patient Positioning In Robotic Surgery,” all of which are hereby incorporated by reference herein in their entireties.

### TECHNICAL FIELD

The systems and methods disclosed herein are directed to robotic medical systems, and more particularly to robotic medical systems that include patient platform systems (e.g., a surgical table or bed).

### BACKGROUND

Patient platform systems can be used in robotic medical procedures, such as an imaging procedure or a surgical procedure, to support a patient. The position of a table top of the patient platform system may be adjusted during a medical procedure to improve visibility or accessibility of a particular anatomical part of the patient.

In certain procedures, robotic arms of a robotic medical system may be used to control placement, insertion, and/or manipulation of one or more medical tools. However, when such robotic arms are used for a patient positioned on a patient platform system, the robotic arms are retrieved during repositioning of the patient, which can interrupt imaging or surgical procedures.

### SUMMARY

Disclosed herein is a patient platform system that provides coordinated motion between movement of the patient platform system and robotic arms of a robotic medical system that is coupled to or used in conjunction with the patient platform system so that retrieval of the robotic arms during the patient repositioning is reduced or eliminated.

A patient platform system with a table and one or more kinematic chains is configurable to perform a variety of surgical or medical procedures on a patient. The table includes a rigid base and a table top that is movable relative to the rigid base in accordance with a user request (e.g., horizontally and/or vertically). The table includes mechanisms that allow the table to be translated relative to the rigid base, and rotated with respect to a transverse axis (pitch) and a longitudinal axis (roll) of the table top. In response to initiating movement of the table top relative to the rigid base in accordance with the user request, the one or more kinematic chains are moved relative to the rigid base in coordination with the movement of the table top such that one or more preset conditions are maintained during the first movement of the table top.

In accordance with some embodiments, a patient platform system includes a table with a rigid base and a table top that is movable relative to the rigid base, one or more kinematic chains that are coupled to the table, one or more processors, and memory storing instructions. The instructions, when executed by the one or more processors, cause the one or more processors to initiate first movement of the table top relative to the rigid base in accordance with a user request, and move the one or more kinematic chains relative to the rigid base in coordination with the first movement of the table top such that one or more preset conditions are maintained during the first movement of the table top.

In some embodiments, the one or more kinematic chains include at least a first robotic arm.

In some embodiments, the one or more preset conditions that are maintained during the first movement of the table top include a preset condition that limits movement of a first distal portion of a first kinematic chain to less than a threshold amount of movement relative to the table top.

In some embodiments, the preset condition that limits the movement of the first distal portion of the first kinematic chain to less than the threshold amount of movement relative to the table top includes maintaining a remote center of motion associated with the first kinematic chain relative to the table top.

In some embodiments, the one or more preset conditions that are maintained during the first movement of the table top include a preset condition that prohibits one or more of: the one or more kinematic chains and the table top from reaching within a threshold distance of one another during the first movement of the table top, or kinematic chains of the one or more kinematic chains from reaching within a threshold distance of one another during the first movement of the table top.

In some embodiments, the one or more preset conditions that are maintained during the first movement of the table top include a preset condition that prevents the one or more kinematic chains from reaching within a threshold distance of one or more objects adjacent to the patient platform system.

In some embodiments, the one or more kinematic chains include a first kinematic chain that includes a first joint. The one or more preset conditions that are maintained during the first movement of the table top include a preset condition that prevents the first joint from reaching beyond a joint limit.

In some embodiments, the one or more kinematic chains include at least a first robotic arm that has an attached medical tool at its distal end during the first movement of the table top.

In some embodiments, the one or more preset conditions that are maintained during the first movement of the table top include a preset condition that maintains a fixed spatial relationship between the attached medical tool and the table top during the first movement of the table top.

In some embodiments, the one or more preset conditions include maintaining an aligned spatial relationship to a tele-operation input device of the attached medical tool relative to the table top.

In some embodiments, the one or more kinematic chains include a first robotic arm and an adjustable arm support on which the first robotic arm is positioned.

In some embodiments, the instructions, when executed by the one or more processors, cause the one or more processors to move the adjustable arm support and the first robotic arm in coordination with the first movement of the table top such that the one or more preset conditions are maintained during the first movement of the table top.

In some embodiments, the one or more kinematic chains include one or more robotic arms that are in an undocked state relative to the table top. The one or more preset conditions permit spatial relationships between the table top and respective distal portions of the one or more robotic arms to change by more than a threshold amount during the first movement of the table top.

In some embodiments, the one or more kinematic chains include one or more robotic arms that are in an undocked state relative to the table top. The one or more undocked robotic arms remain in non-interfering configurations during the first movement of the table top.

In accordance with some embodiments, a method of operating a patient platform is disclosed. The patient platform system includes a table with a rigid base and a table top. The method includes receiving user request to move the table top relative to the rigid base. The method also includes initiating first movement of the table top relative to the rigid base in accordance with the user request, and moving one or more kinematic chains relative to the rigid base in coordination with the first movement of the table top such that one or more preset conditions are maintained during the first movement of the table top, the one or more kinematic chains being coupled to the table.

In accordance with some embodiments, a computer readable storage medium stores one or more programs configured for execution by one or more processors. The one or more programs include instructions for receiving a user request to move a table top of a patient platform system. The patient platform system includes a table with the table top and a rigid base, and the table top is movable relative to a rigid base. The one or more programs also include instructions for initiating first movement of the table top relative to a rigid base in accordance with the user request, and moving one or more kinematic chains relative to the rigid base in coordination with the first movement of the table top such that one or more preset conditions are maintained during the first movement of the table top. The one or more kinematic chains are coupled to the table.

In accordance with some embodiments, a patient platform system includes a table with a rigid base and a table top that is movable relative to the rigid base, a first robotic arm that is coupled to the table, one or more processors, and memory storing instructions. The instructions, when executed by the one or more processors, cause the one or more processors to initiate first movement of the table top relative to the rigid base, and constrain a change in spatial relationship between a first distal portion of the first robotic arm and the table top during the first movement of the table top.

In some embodiments, constraining the change in spatial relationship between the first distal portion of the first robotic arm and the table top during the first movement of the table top includes, in accordance with the first movement of the table top, moving at least a portion of the first robotic arm relative to the table top in a manner that limits movement of the first distal portion of the first robotic arm to less than a threshold amount of movement relative to the table top.

In some embodiments, constraining the change in spatial relationship between the first distal portion of the first robotic arm and the table top during the first movement of the table top includes maintaining a remote center of motion associated with the first robotic arm relative to the table top.

In some embodiments, the patient platform system further includes an adjustable arm support, and the first robotic arm is movably coupled to the adjustable arm support. Constraining the change in spatial relationship between a first distal end of the first robotic arm and the table top during the first movement of the table top includes, in accordance with the first movement of the table top, moving the adjustable arm support relative to the table top in a manner that limits movement of the first distal portion of the first robotic arm to less than a threshold amount of movement relative to the table top.

In some embodiments, the patient platform system further includes an adjustable arm support, and the first robotic arm is movably coupled to the adjustable arm support. Constraining the change in spatial relationship between a first distal end of the first robotic arm and the table top during the first movement of the table top includes, in accordance with the first movement of the table top, coordinating movement of the first robotic arm relative to the adjustable arm support and movement of the adjustable arm support relative to the table top in a manner that limits movement of the first distal portion of the first robotic arm to less than a threshold amount of movement relative to the table top.

In some embodiments, the first robotic arm includes at least one kinematically redundant joint that is configured to move while the change in spatial relationship between the first distal portion of the first robotic arm and the table top is constrained.

In some embodiments, the patient platform system further includes a second robotic arm in addition to the first robotic arm. The stored instructions, when executed by the one or more processors, cause the one or more processors to perform any of (a) moving any of the first robotic arm and the second robotic arm in a coordinated manner such that collision between the first robotic arm and the second robotic arm is avoided during the first movement of the table top, (b) moving any of the first robotic arm and the second robotic arm to avoid self-collision, (c) moving any of the first robotic arm and the second robotic arm for joint limit avoidance, and (d) moving any of the first robotic arm and the second robotic arm to avoid collision with any of the table and one or more structures that are operable coupled to the table.

In some embodiments, the patient platform system further includes an adjustable arm support configured to support at least one of the first robotic arm or the second robotic arm. The stored instructions, when executed by the one or more processors, cause the one or more processors to perform any of (e) moving the adjustable arm support and at least one of the first robotic arm or the second robotic arm to avoid collision with any of the table and one or more structures that are operable coupled to the table, and (f) moving the adjustable arm support and at least one of the first robotic arm or the second robotic arm to avoid collision with a ground that supports the table.

In accordance with some embodiments, a method of operating a patient platform system is disclosed. The patient platform system includes a table with a rigid base and a table top. The method includes initiating first movement of a table top relative to a rigid base, and constraining a change in spatial relationship between a first distal portion of a first robotic arm and the table top during the first movement of the table top. The first robotic arm is coupled to the table.

In accordance with some embodiments, a non-transitory computer readable storage medium stores one or more programs configured for execution by one or more processors. The one or more programs include instructions for initiating first movement of a table top of a patient platform system. The patient platform system includes a table with a rigid base and the table top, and the table top is movable relative to the rigid base. The one or more programs also include instructions for constraining a change in spatial relationship between a first distal portion of a first robotic arm and the table top during the first movement of the table top. The first robotic arm is coupled to the table.

In accordance with some embodiments, a patient platform system includes a table with a rigid base and a table top that is movable relative to the rigid base, a first robotic arm, one or more sensors that are positioned to detect one or more forces exerted on the first robotic arm, one or more processor, and memory storing instructions. The instructions, when executed by the one or more processors, cause the one or more processors to initiate first movement of the table top relative to the rigid base, move the first robotic arm in coordination with the first movement of the table top, and obtain sensor information from the one or more sensors. The sensor information includes information regarding one or more forces exerted on the first robotic arm during the first movement of the table top and movement of the first robotic arm in coordination with the first movement of the table top.

In some embodiments, the one or more forces exerted on the first robotic arm include a force component associated with gravity of a patient positioned on the table top.

In some embodiments, the stored instructions, when executed by the one or more processors, cause the one or more processors to, in accordance with the sensor information, constrain a change in spatial relationship between a first distal portion of the first robotic arm and the table top during the first movement of the table top.

In some embodiments, constraining the change in spatial relationship between the first distal portion of the first robotic arm and the table top during the first movement of the table top includes: in accordance with a determination that the sensor information meets first criteria, constraining movement of the first distal portion of the first robotic arm relative to the table top based on a first constraint, and in accordance with a determination that the sensor information does not meet the first criteria, constraining movement of the first distal portion of the first robotic arm relative to the table top based on a second constraint that is different from the first constraint.

In some embodiments, the first robotic arm includes an attached surgical tool that is retracted from the first distal portion of the first robotic arm away from the table top.

In some embodiments, the one or more forces exerted on the first robotic arm include a force component associated with impact from an object external to the patient platform system.

In some embodiments, the stored instructions further include instructions, which, when executed by the one or more processors, cause the one or more processors to, in accordance with the sensor information, activate power-assisted movement of the first robotic arm during the first movement of the table top.

In accordance with some embodiments, a method of operating the patient platform system is disclosed. The patient platform system includes a first robotic arm, a table with a rigid base and a table top, and one or more sensors positioned to detect one or more forces exerted on the first robotic arm. The method includes initiating first movement of the table top relative to the rigid base, and moving the first robotic arm in coordination with the first movement of the table top. The method also includes obtaining, from the one or more sensors, sensor information sensor regarding one or more forces exerted on the first robotic arm during the first movement of the table top and movement of the first robotic arm in coordination with the first movement of the table top.

In accordance with some embodiments, a computer readable storage medium stores one or more programs configured for execution by one or more processors. The one or more programs include instructions for initiating first movement of a table top of a patient platform system. The patient platform system includes a first robotic arm, a table with a rigid base and a table top, and one or more sensors positioned to detect one or more forces exerted on the first robotic arm. The table top is movable relative to the rigid base. The one or more programs also include instructions for moving the first robotic arm in coordination with the first movement of the table top, and obtaining sensor information from one or more sensors that are positioned to detect one or more forces exerted on the first robotic arm. The sensor information includes information regarding one or more forces exerted on the first robotic arm during the first movement of the table top and movement of the first robotic arm in coordination with the first movement of the table top.

In accordance with some embodiments, a patient platform system includes a table with a rigid base and a table top that is movable relative to the rigid base, a first robotic arm, one or more processors, and memory storing instructions. The instructions, when executed by the one or more processors, cause the one or more processors to move the first robotic arm in coordination with first movement of the table top relative to the rigid base and halt at least one of movement of first robotic arm or the first movement of the table top in accordance with a determination that one or more criteria are met.

In some embodiments, halting the at least one of the movement of first robotic arm or the first movement of the table top in accordance with the determination that the one or more criteria are met includes halting the at least one of the movement of first robotic arm or the first movement of the table top in accordance with receipt of a user input corresponding to a request to halt the at least one of the movement of the first robotic arm or the first movement of the table top.

In some embodiments, halting the at least one of the movement of first robotic arm or the first movement of the table top in accordance with the determination that the one or more criteria are met includes halting the at least one of the movement of first robotic arm or the first movement of the table top in accordance with detection of a collision with the first robotic arm or the table top or anticipation of a collision with the first robotic arm or the table top that are not resolvable with permitted movement of the first robotic arm or the table top.

In some embodiments, halting the at least one of the movement of first robotic arm or the first movement of the table top in accordance with a determination that the one or more criteria are met includes halting the at least one of the movement of first robotic arm or the first movement of the table top in accordance with determining that at least one of the first robotic arm or the table top has reached an associated joint limit.

In some embodiments, halting the at least one of the movement of first robotic arm or the first movement of the table top in accordance with a determination that the one or more criteria are met includes halting the at least one of the movement of first robotic arm or the first movement of the table top in accordance with detecting that a force exerted on the first robotic arm has exceeded a preset force threshold.

In some embodiments, the patient platform system further includes a display. The stored instructions further include instructions, which, when executed by the one or more processors, cause the one or more processors to, after halting the at least one of movement of the first robotic arm or the first movement of the table top and in accordance with the determination that one or more criteria are met, present information regarding the one or more criteria that are met at the display, and/or present a graphical user interface for user intervention of movement of the first robotic arm and/or movement of the table top.

In accordance with some embodiments, a method of operating a patient platform system is disclosed. The patient platform system includes a first robotic arm and a table with a rigid base and a table top. The method includes moving the first robotic arm in coordination with first movement of the table top relative to the rigid base, and halting at least one of movement of first robotic arm or the first movement of the table top in accordance with a determination that one or more criteria are met.

In accordance with some embodiments, a computer readable storage medium stores one or more programs configured for execution by one or more processors. The one or more programs include instructions for moving a first robotic arm in coordination with first movement of a table top relative to a rigid base, and halting at least one of movement of first robotic arm or the first movement of the table top in accordance with a determination that one or more criteria are met.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an embodiment of a cart-based robotic system arranged for diagnostic and/or therapeutic bronchoscopy procedure(s).

FIG. 2 depicts further aspects of the robotic system of FIG. 1.

FIG. 3 illustrates an embodiment of the robotic system of FIG. 1 arranged for ureteroscopy.

FIG. 4 illustrates an embodiment of the robotic system of FIG. 1 arranged for a vascular procedure.

FIG. 5 illustrates an embodiment of a table-based robotic system arranged for a bronchoscopy procedure.

FIG. 6 provides an alternative view of the robotic system of FIG. 5.

FIG. 7 illustrates an example system configured to stow robotic arm(s).

FIG. 8 illustrates an embodiment of a table-based robotic system configured for a ureteroscopy procedure.

FIG. 9 illustrates an embodiment of a table-based robotic system configured for a laparoscopic procedure.

FIG. 10 illustrates an embodiment of the table-based robotic system of FIGS. 5-9 with pitch or tilt adjustment.

FIG. 11 provides a detailed illustration of the interface between the table and the column of the table-based robotic system of FIGS. 5-10.

FIG. 12 illustrates an alternative embodiment of a table-based robotic system.

FIG. 13 illustrates an end view of the table-based robotic system of FIG. 12.

FIG. 14 illustrates an end view of a table-based robotic system with robotic arms attached thereto.

FIG. 15 illustrates an exemplary instrument driver.

FIG. 16 illustrates an exemplary medical instrument with a paired instrument driver.

FIG. 17 illustrates an alternative design for an instrument driver and instrument where the axes of the drive units are parallel to the axis of the elongated shaft of the instrument.

FIG. 18 illustrates an instrument having an instrument-based insertion architecture.

FIG. 19 illustrates an exemplary controller.

FIG. 20 depicts a block diagram illustrating a localization system that estimates a location of one or more elements of the robotic systems of FIGS. 1-10, such as the location of the instrument of FIGS. 16-18, in accordance to an example embodiment.

FIG. 21 illustrates a patient platform system in accordance with some embodiments.

FIGS. 22A and 22B illustrate translation of the table top of the patient platform system of FIG. 21 in accordance with some embodiments.

FIGS. 22C and 22D illustrate rotation of the table top of the patient platform system of FIG. 21 in accordance with some embodiments.

FIG. 23 illustrates a robotic arm of the patient platform system of FIG. 21 in accordance with some embodiments.

FIGS. 24A-24C illustrate an example of coordinated motion between a table top and robotic arms of the patient platform system of FIG. 21 in accordance with some embodiments.

FIGS. 25A-25C illustrate another example of coordinated motion between a table top and robotic arms of the patient platform system of FIG. 21 in accordance with some embodiments.

FIG. 26 illustrates an input device for controlling movement of the patient platform of FIG. 21 in accordance with some embodiments.

FIGS. 27A-27E show a flowchart for operating the patient platform system of FIG. 21 in accordance with some embodiments.

FIGS. 28A-28D show a flow diagram illustrating a method of performing coordinated motion by a patient platform system in accordance with some embodiments.

FIGS. 29A-29C show a flow diagram illustrating a method of operating a patient platform system in accordance with some embodiments.

FIG. 30 shows a flow diagram illustrating a method of operating a patient platform system in accordance with some embodiments.

FIGS. 31A-31B show a flow diagram illustrating a method of operating a patient platform system in accordance with some embodiments.

FIG. 32 is a schematic diagram illustrating electronic components of the patient platform system in accordance with some embodiments.

### DETAILED DESCRIPTION

### 1. Overview

Aspects of the present disclosure may be integrated into a robotically-enabled medical system capable of performing a variety of medical procedures, including both minimally invasive, such as laparoscopy, and non-invasive, such as endoscopy, procedures. Among endoscopy procedures, the system may be capable of performing bronchoscopy, ureteroscopy, gastroscopy, etc.

In addition to performing the breadth of procedures, the system may provide additional benefits, such as enhanced imaging and guidance to assist the physician. Additionally, the system may provide the physician with the ability to perform the procedure from an ergonomic position without the need for awkward arm motions and positions. Still further, the system may provide the physician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification.

### A. Robotic System—Cart.

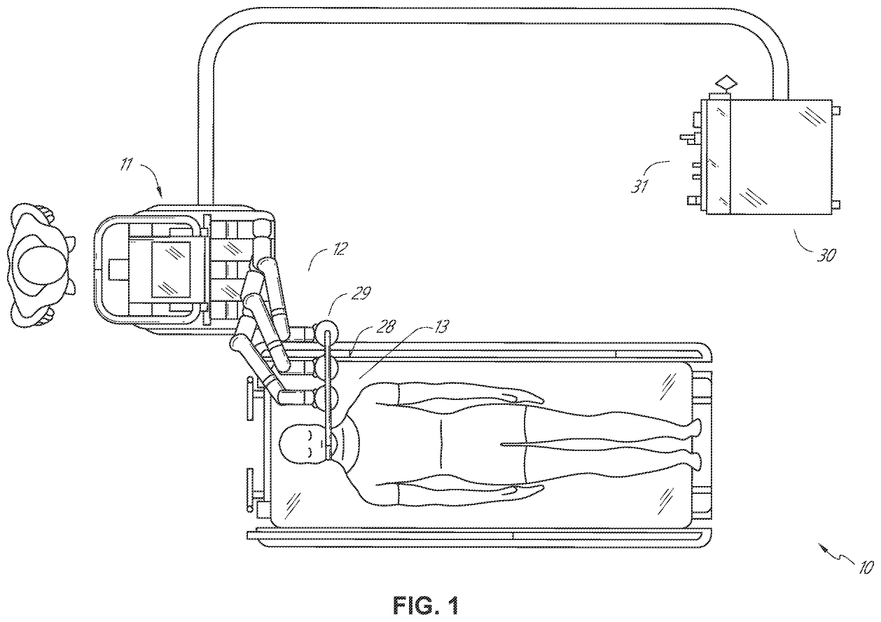

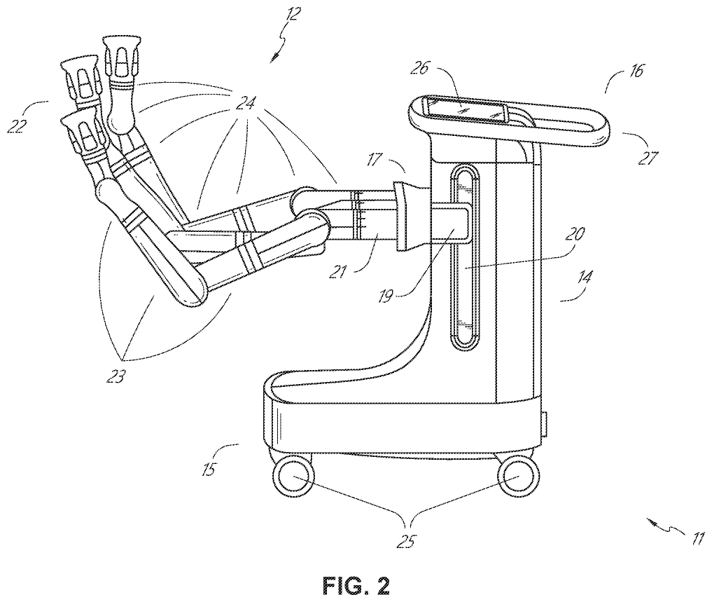

The robotically-enabled medical system may be configured in a variety of ways depending on the particular procedure. FIG. 1 illustrates an embodiment of a cart-based robotically-enabled system 10 arranged for a diagnostic and/or therapeutic bronchoscopy procedure. During a bronchoscopy, the system 10 may comprise a cart 11 having one or more robotic arms 12 to deliver a medical instrument, such as a steerable endoscope 13, which may be a procedure-specific bronchoscope for bronchoscopy, to a natural orifice access point (i.e., the mouth of the patient positioned on a table in the present example) to deliver diagnostic and/or therapeutic tools. As shown, the cart 11 may be positioned proximate to the patient's upper torso in order to provide access to the access point. Similarly, the robotic arms 12 may be actuated to position the bronchoscope relative to the access point. The arrangement in FIG. 1 may also be utilized when performing a gastro-intestinal (GI) procedure with a gastroscope, a specialized endoscope for GI procedures. FIG. 2 depicts an example embodiment of the cart in greater detail.

With continued reference to FIG. 1, once the cart 11 is properly positioned, the robotic arms 12 may insert the steerable endoscope 13 into the patient robotically, manually, or a combination thereof. As shown, the steerable endoscope 13 may comprise at least two telescoping parts, such as an inner leader portion and an outer sheath portion, each portion coupled to a separate instrument driver from the set of instrument drivers 28, each instrument driver coupled to the distal end of an individual robotic arm. This linear arrangement of the instrument drivers 28, which facilitates coaxially aligning the leader portion with the sheath portion, creates a “virtual rail” 29 that may be repositioned in space by manipulating the one or more robotic arms 12 into different angles and/or positions. The virtual rails described herein are depicted in the Figures using dashed lines, and accordingly the dashed lines do not depict any physical structure of the system. Translation of the instrument drivers 28 along the virtual rail 29 telescopes the inner leader portion relative to the outer sheath portion or advances or retracts the endoscope 13 from the patient. The angle of the virtual rail 29 may be adjusted, translated, and pivoted based on clinical application or physician preference. For example, in bronchoscopy, the angle and position of the virtual rail 29 as shown represents a compromise between providing physician access to the endoscope 13 while minimizing friction that results from bending the endoscope 13 into the patient's mouth.

The endoscope 13 may be directed down the patient's trachea and lungs after insertion using precise commands from the robotic system until reaching the target destination or operative site. In order to enhance navigation through the patient's lung network and/or reach the desired target, the endoscope 13 may be manipulated to telescopically extend the inner leader portion from the outer sheath portion to obtain enhanced articulation and greater bend radius. The use of separate instrument drivers 28 also allows the leader portion and sheath portion to be driven independent of each other.

For example, the endoscope 13 may be directed to deliver a biopsy needle to a target, such as, for example, a lesion or nodule within the lungs of a patient. The needle may be deployed down a working channel that runs the length of the endoscope to obtain a tissue sample to be analyzed by a pathologist. Depending on the pathology results, additional tools may be deployed down the working channel of the endoscope for additional biopsies. After identifying a nodule to be malignant, the endoscope 13 may endoscopically deliver tools to resect the potentially cancerous tissue. In some instances, diagnostic and therapeutic treatments can be delivered in separate procedures. In those circumstances, the endoscope 13 may also be used to deliver a fiducial to “mark” the location of the target nodule as well. In other instances, diagnostic and therapeutic treatments may be delivered during the same procedure.

The system 10 may also include a movable tower 30, which may be connected via support cables to the cart 11 to provide support for controls, electronics, fluidics, optics, sensors, and/or power to the cart 11. Placing such functionality in the tower 30 allows for a smaller form factor cart 11 that may be more easily adjusted and/or re-positioned by an operating physician and his/her staff. Additionally, the division of functionality between the cart/table and the support tower 30 reduces operating room clutter and facilitates improving clinical workflow. While the cart 11 may be positioned close to the patient, the tower 30 may be stowed in a remote location to stay out of the way during a procedure.

In support of the robotic systems described above, the tower 30 may include component(s) of a computer-based control system that stores computer program instructions, for example, within a non-transitory computer-readable storage medium such as a persistent magnetic storage drive, solid state drive, etc. The execution of those instructions, whether the execution occurs in the tower 30 or the cart 11, may control the entire system or sub-system(s) thereof. For example, when executed by a processor of the computer system, the instructions may cause the components of the robotics system to actuate the relevant carriages and arm mounts, actuate the robotics arms, and control the medical instruments. For example, in response to receiving the control signal, the motors in the joints of the robotics arms may position the arms into a certain posture.

The tower 30 may also include a pump, flow meter, valve control, and/or fluid access in order to provide controlled irrigation and aspiration capabilities to the system that may be deployed through the endoscope 13. These components may also be controlled using the computer system of tower 30. In some embodiments, irrigation and aspiration capabilities may be delivered directly to the endoscope 13 through separate cable(s).

The tower 30 may include a voltage and surge protector designed to provide filtered and protected electrical power to the cart 11, thereby avoiding placement of a power transformer and other auxiliary power components in the cart 11, resulting in a smaller, more moveable cart 11.

The tower 30 may also include support equipment for the sensors deployed throughout the robotic system 10. For example, the tower 30 may include opto-electronics equipment for detecting, receiving, and processing data received from the optical sensors or cameras throughout the robotic system 10. In combination with the control system, such opto-electronics equipment may be used to generate real-time images for display in any number of consoles deployed throughout the system, including in the tower 30. Similarly, the tower 30 may also include an electronic subsystem for receiving and processing signals received from deployed electromagnetic (EM) sensors. The tower 30 may also be used to house and position an EM field generator for detection by EM sensors in or on the medical instrument.

The tower 30 may also include a console 31 in addition to other consoles available in the rest of the system, e.g., console mounted on top of the cart. The console 31 may include a user interface and a display screen, such as a touchscreen, for the physician operator. Consoles in system 10 are generally designed to provide both robotic controls as well as pre-operative and real-time information of the procedure, such as navigational and localization information of the endoscope 13. When the console 31 is not the only console available to the physician, it may be used by a second operator, such as a nurse, to monitor the health or vitals of the patient and the operation of system, as well as provide procedure-specific data, such as navigational and localization information. In other embodiments, the console 31 is housed in a body that is separate from the tower 30.

The tower 30 may be coupled to the cart 11 and endoscope 13 through one or more cables or connections (not shown). In some embodiments, the support functionality from the tower 30 may be provided through a single cable to the cart 11, simplifying and de-cluttering the operating room. In other embodiments, specific functionality may be coupled in separate cabling and connections. For example, while power may be provided through a single power cable to the cart, the support for controls, optics, fluidics, and/or navigation may be provided through a separate cable.

FIG. 2 provides a detailed illustration of an embodiment of the cart from the cart-based robotically-enabled system shown in FIG. 1. The cart 11 generally includes an elongated support structure 14 (often referred to as a “column”), a cart base 15, and a console 16 at the top of the column 14. The column 14 may include one or more carriages, such as a carriage 17 (alternatively “arm support”) for supporting the deployment of one or more robotic arms 12 (three shown in FIG. 2). The carriage 17 may include individually configurable arm mounts that rotate along a perpendicular axis to adjust the base of the robotic arms 12 for better positioning relative to the patient. The carriage 17 also includes a carriage interface 19 that allows the carriage 17 to vertically translate along the column 14.

The carriage interface 19 is connected to the column 14 through slots, such as slot 20, that are positioned on opposite sides of the column 14 to guide the vertical translation of the carriage 17. The slot 20 contains a vertical translation interface to position and hold the carriage at various vertical heights relative to the cart base 15. Vertical translation of the carriage 17 allows the cart 11 to adjust the reach of the robotic arms 12 to meet a variety of table heights, patient sizes, and physician preferences. Similarly, the individually configurable arm mounts on the carriage 17 allow the robotic arm base 21 of robotic arms 12 to be angled in a variety of configurations.

In some embodiments, the slot 20 may be supplemented with slot covers that are flush and parallel to the slot surface to prevent dirt and fluid ingress into the internal chambers of the column 14 and the vertical translation interface as the carriage 17 vertically translates. The slot covers may be deployed through pairs of spring spools positioned near the vertical top and bottom of the slot 20. The covers are coiled within the spools until deployed to extend and retract from their coiled state as the carriage 17 vertically translates up and down. The spring-loading of the spools provides force to retract the cover into a spool when carriage 17 translates towards the spool, while also maintaining a tight seal when the carriage 17 translates away from the spool. The covers may be connected to the carriage 17 using, for example, brackets in the carriage interface 19 to ensure proper extension and retraction of the cover as the carriage 17 translates.

The column 14 may internally comprise mechanisms, such as gears and motors, that are designed to use a vertically aligned lead screw to translate the carriage 17 in a mechanized fashion in response to control signals generated in response to user inputs, e.g., inputs from the console 16.

The robotic arms 12 may generally comprise robotic arm bases 21 and end effectors 22, separated by a series of linkages 23 that are connected by a series of joints 24, each joint comprising an independent actuator, each actuator comprising an independently controllable motor. Each independently controllable joint represents an independent degree of freedom available to the robotic arm. Each of the arms 12 have seven joints, and thus provide seven degrees of freedom. A multitude of joints result in a multitude of degrees of freedom, allowing for “redundant” degrees of freedom. Redundant degrees of freedom allow the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The cart base 15 balances the weight of the column 14, carriage 17, and arms 12 over the floor. Accordingly, the cart base 15 houses heavier components, such as electronics, motors, power supply, as well as components that either enable movement and/or immobilize the cart. For example, the cart base 15 includes rollable wheel-shaped casters 25 that allow for the cart to easily move around the room prior to a procedure. After reaching the appropriate position, the casters 25 may be immobilized using wheel locks to hold the cart 11 in place during the procedure.

Positioned at the vertical end of column 14, the console 16 allows for both a user interface for receiving user input and a display screen (or a dual-purpose device such as, for example, a touchscreen 26) to provide the physician user with both pre-operative and intra-operative data. Potential pre-operative data on the touchscreen 26 may include pre-operative plans, navigation and mapping data derived from pre-operative computerized tomography (CT) scans, and/or notes from pre-operative patient interviews. Intra-operative data on display may include optical information provided from the tool, sensor and coordinate information from sensors, as well as vital patient statistics, such as respiration, heart rate, and/or pulse. The console 16 may be positioned and tilted to allow a physician to access the console from the side of the column 14 opposite carriage 17. From this position, the physician may view the console 16, robotic arms 12, and patient while operating the console 16 from behind the cart 11. As shown, the console 16 also includes a handle 27 to assist with maneuvering and stabilizing cart 11.

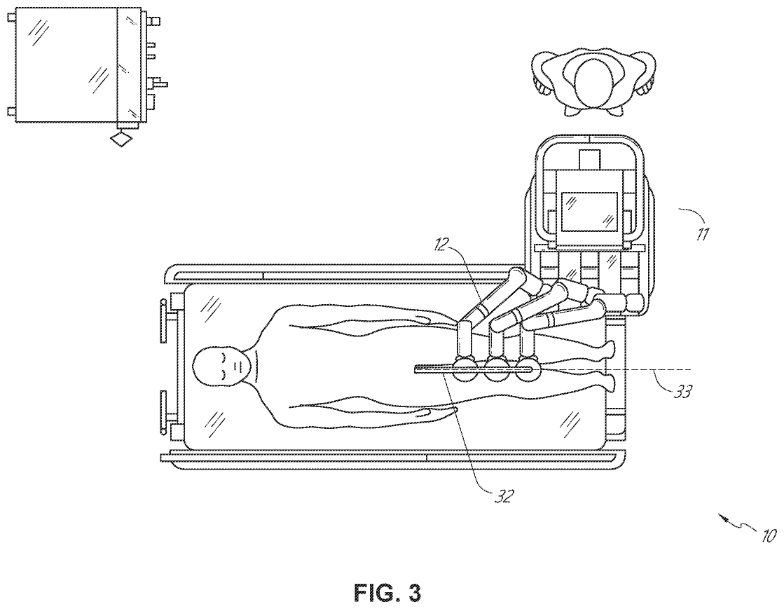

FIG. 3 illustrates an embodiment of a robotically-enabled system 10 arranged for ureteroscopy. In a ureteroscopic procedure, the cart 11 may be positioned to deliver a ureteroscope 32, a procedure-specific endoscope designed to traverse a patient's urethra and ureter, to the lower abdominal area of the patient. In a ureteroscopy, it may be desirable for the ureteroscope 32 to be directly aligned with the patient's urethra to reduce friction and forces on the sensitive anatomy in the area. As shown, the cart 11 may be aligned at the foot of the table to allow the robotic arms 12 to position the ureteroscope 32 for direct linear access to the patient's urethra. From the foot of the table, the robotic arms 12 may insert the ureteroscope 32 along the virtual rail 33 directly into the patient's lower abdomen through the urethra.

After insertion into the urethra, using similar control techniques as in bronchoscopy, the ureteroscope 32 may be navigated into the bladder, ureters, and/or kidneys for diagnostic and/or therapeutic applications. For example, the ureteroscope 32 may be directed into the ureter and kidneys to break up kidney stone build up using a laser or ultrasonic lithotripsy device deployed down the working channel of the ureteroscope 32. After lithotripsy is complete, the resulting stone fragments may be removed using baskets deployed down the ureteroscope 32.

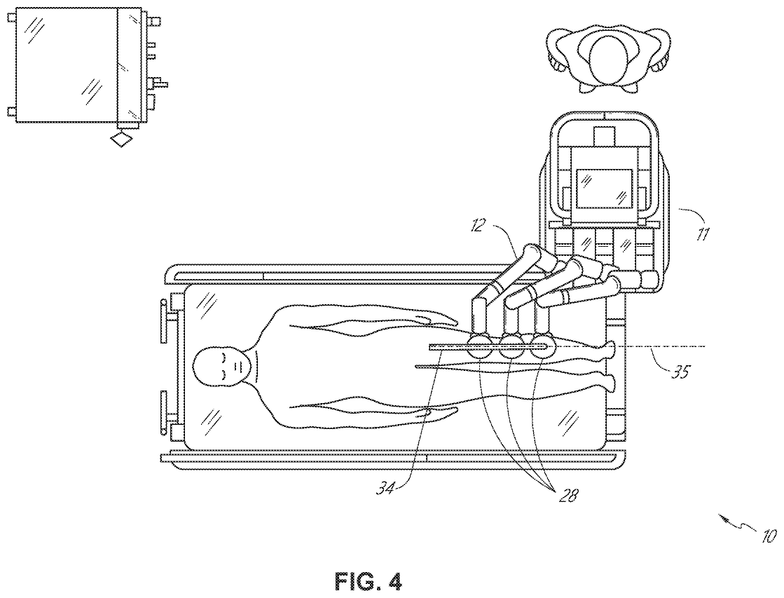

FIG. 4 illustrates an embodiment of a robotically-enabled system similarly arranged for a vascular procedure. In a vascular procedure, the system 10 may be configured such that the cart 11 may deliver a medical instrument 34, such as a steerable catheter, to an access point in the femoral artery in the patient's leg. The femoral artery presents both a larger diameter for navigation as well as a relatively less circuitous and tortuous path to the patient's heart, which simplifies navigation. As in a ureteroscopic procedure, the cart 11 may be positioned towards the patient's legs and lower abdomen to allow the robotic arms 12 to provide a virtual rail 35 with direct linear access to the femoral artery access point in the patient's thigh/hip region. After insertion into the artery, the medical instrument 34 may be directed and inserted by translating the instrument drivers 28. Alternatively, the cart may be positioned around the patient's upper abdomen in order to reach alternative vascular access points, such as, for example, the carotid and brachial arteries near the shoulder and wrist.

### B. Robotic System—Table.

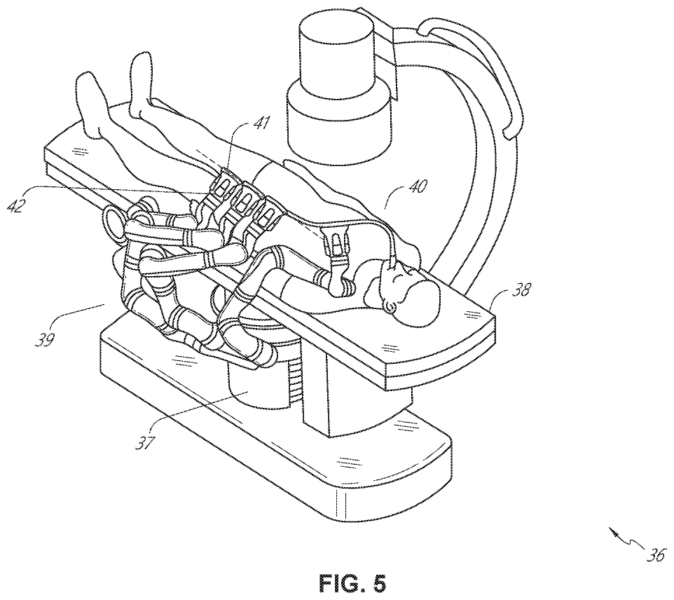

Embodiments of the robotically-enabled medical system may also incorporate the patient's table. Incorporation of the table reduces the amount of capital equipment within the operating room by removing the cart, which allows greater access to the patient. FIG. 5 illustrates an embodiment of such a robotically-enabled system arranged for a bronchoscopy procedure. System 36 includes a support structure or column 37 for supporting platform 38 (shown as a “table” or “bed”) over the floor. Much like in the cart-based systems, the end effectors of the robotic arms 39 of the system 36 comprise instrument drivers 42 that are designed to manipulate an elongated medical instrument, such as a bronchoscope 40 in FIG. 5, through or along a virtual rail 41 formed from the linear alignment of the instrument drivers 42. In practice, a C-arm for providing fluoroscopic imaging may be positioned over the patient's upper abdominal area by placing the emitter and detector around table 38.

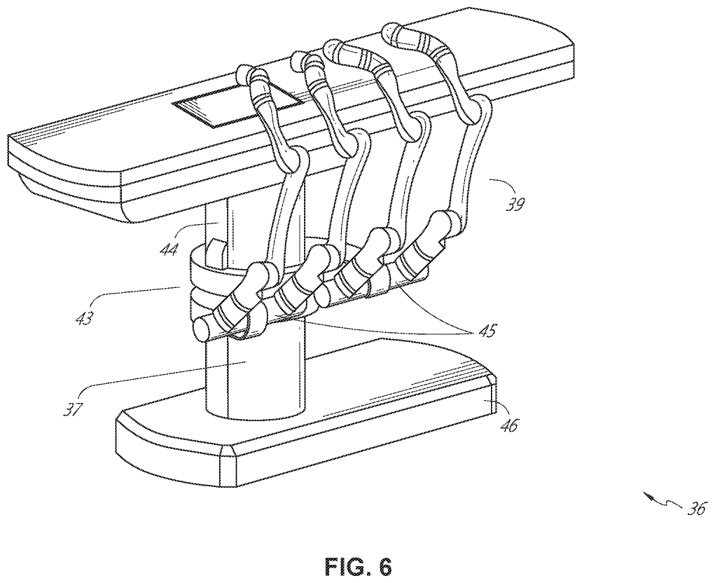

FIG. 6 provides an alternative view of the system 36 without the patient and medical instrument for discussion purposes. As shown, the column 37 may include one or more carriages 43 shown as ring-shaped in the system 36, from which the one or more robotic arms 39 may be based. The carriages 43 may translate along a vertical column interface 44 that runs the length of the column 37 to provide different vantage points from which the robotic arms 39 may be positioned to reach the patient. The carriage(s) 43 may rotate around the column 37 using a mechanical motor positioned within the column 37 to allow the robotic arms 39 to have access to multiples sides of the table 38, such as, for example, both sides of the patient. In embodiments with multiple carriages, the carriages may be individually positioned on the column and may translate and/or rotate independent of the other carriages. While carriages 43 need not surround the column 37 or even be circular, the ring-shape as shown facilitates rotation of the carriages 43 around the column 37 while maintaining structural balance. Rotation and translation of the carriages 43 allows the system to align the medical instruments, such as endoscopes and laparoscopes, into different access points on the patient. In other embodiments (not shown), the system 36 can include a patient table or bed with adjustable arm supports in the form of bars or rails extending alongside it. One or more robotic arms 39 (e.g., via a shoulder with an elbow joint) can be attached to the adjustable arm supports, which can be vertically adjusted. By providing vertical adjustment, the robotic arms 39 are advantageously capable of being stowed compactly beneath the patient table or bed, and subsequently raised during a procedure.

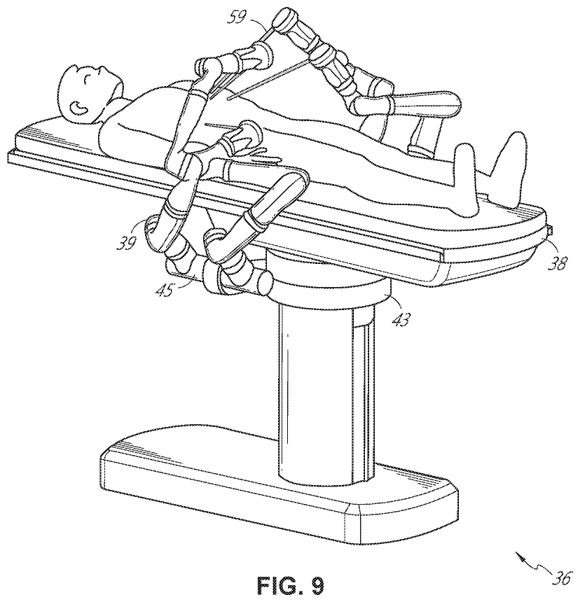

The arms 39 may be mounted on the carriages through a set of arm mounts 45 comprising a series of joints that may individually rotate and/or telescopically extend to provide additional configurability to the robotic arms 39. Additionally, the arm mounts 45 may be positioned on the carriages 43 such that, when the carriages 43 are appropriately rotated, the arm mounts 45 may be positioned on either the same side of table 38 (as shown in FIG. 6), on opposite sides of table 38 (as shown in FIG. 9), or on adjacent sides of the table 38 (not shown).

The column 37 structurally provides support for the table 38, and a path for vertical translation of the carriages. Internally, the column 37 may be equipped with lead screws for guiding vertical translation of the carriages, and motors to mechanize the translation of said carriages based the lead screws. The column 37 may also convey power and control signals to the carriage 43 and robotic arms 39 mounted thereon.

The table base 46 serves a similar function as the cart base 15 in cart 11 shown in FIG. 2, housing heavier components to balance the table/bed 38, the column 37, the carriages 43, and the robotic arms 39. The table base 46 may also incorporate rigid casters to provide stability during procedures. Deployed from the bottom of the table base 46, the casters may extend in opposite directions on both sides of the base 46 and retract when the system 36 needs to be moved.

Continuing with FIG. 6, the system 36 may also include a tower (not shown) that divides the functionality of system 36 between table and tower to reduce the form factor and bulk of the table. As in earlier disclosed embodiments, the tower may provide a variety of support functionalities to table, such as processing, computing, and control capabilities, power, fluidics, and/or optical and sensor processing. The tower may also be movable to be positioned away from the patient to improve physician access and de-clutter the operating room. Additionally, placing components in the tower allows for more storage space in the table base for potential stowage of the robotic arms. The tower may also include a master controller or console that provides both a user interface for user input, such as keyboard and/or pendant, as well as a display screen (or touchscreen) for pre-operative and intra-operative information, such as real-time imaging, navigation, and tracking information. In some embodiments, the tower may also contain holders for gas tanks to be used for insufflation.

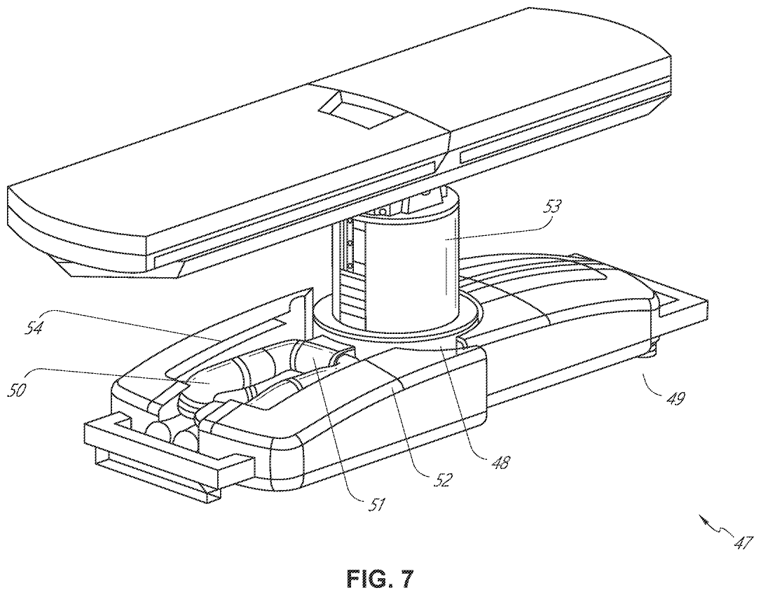

In some embodiments, a table base may stow and store the robotic arms when not in use. FIG. 7 illustrates a system 47 that stows robotic arms in an embodiment of the table-based system. In system 47, carriages 48 may be vertically translated into base 49 to stow robotic arms 50, arm mounts 51, and the carriages 48 within the base 49. Base covers 52 may be translated and retracted open to deploy the carriages 48, arm mounts 51, and arms 50 around column 53, and closed to stow to protect them when not in use. The base covers 52 may be sealed with a membrane 54 along the edges of its opening to prevent dirt and fluid ingress when closed.

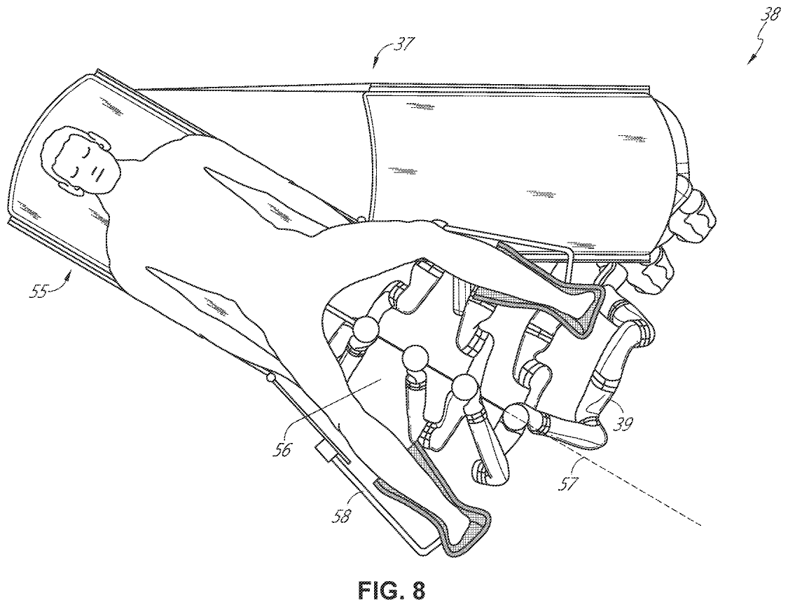

FIG. 8 illustrates an embodiment of a robotically-enabled table-based system configured for a ureteroscopy procedure. In a ureteroscopy, the table 38 may include a swivel portion 55 for positioning a patient off-angle from the column 37 and table base 46. The swivel portion 55 may rotate or pivot around a pivot point (e.g., located below the patient's head) in order to position the bottom portion of the swivel portion 55 away from the column 37. For example, the pivoting of the swivel portion 55 allows a C-arm (not shown) to be positioned over the patient's lower abdomen without competing for space with the column (not shown) below table 38. By rotating the carriage 35 (not shown) around the column 37, the robotic arms 39 may directly insert a ureteroscope 56 along a virtual rail 57 into the patient's groin area to reach the urethra. In a ureteroscopy, stirrups 58 may also be fixed to the swivel portion 55 of the table 38 to support the position of the patient's legs during the procedure and allow clear access to the patient's groin area.

In a laparoscopic procedure, through small incision(s) in the patient's abdominal wall, minimally invasive instruments may be inserted into the patient's anatomy. In some embodiments, the minimally invasive instruments comprise an elongated rigid member, such as a shaft, which is used to access anatomy within the patient. After inflation of the patient's abdominal cavity, the instruments may be directed to perform surgical or medical tasks, such as grasping, cutting, ablating, suturing, etc. In some embodiments, the instruments can comprise a scope, such as a laparoscope. FIG. 9 illustrates an embodiment of a robotically-enabled table-based system configured for a laparoscopic procedure. As shown in FIG. 9, the carriages 43 of the system 36 may be rotated and vertically adjusted to position pairs of the robotic arms 39 on opposite sides of the table 38, such that instrument 59 may be positioned using the arm mounts 45 to be passed through minimal incisions on both sides of the patient to reach his/her abdominal cavity.

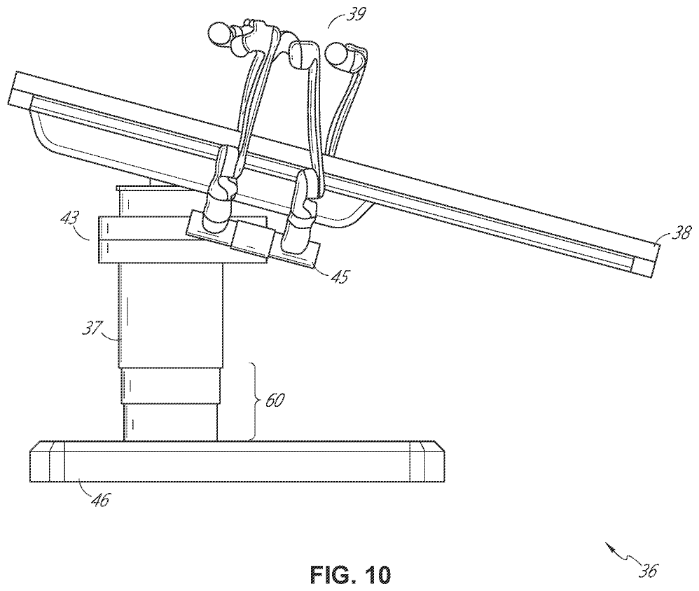

To accommodate laparoscopic procedures, the robotically-enabled table system may also tilt the platform to a desired angle. FIG. 10 illustrates an embodiment of the robotically-enabled medical system with pitch or tilt adjustment. As shown in FIG. 10, the system 36 may accommodate tilt of the table 38 to position one portion of the table at a greater distance from the floor than the other. Additionally, the arm mounts 45 may rotate to match the tilt such that the arms 39 maintain the same planar relationship with table 38. To accommodate steeper angles, the column 37 may also include telescoping portions 60 that allow vertical extension of column 37 to keep the table 38 from touching the floor or colliding with base 46.

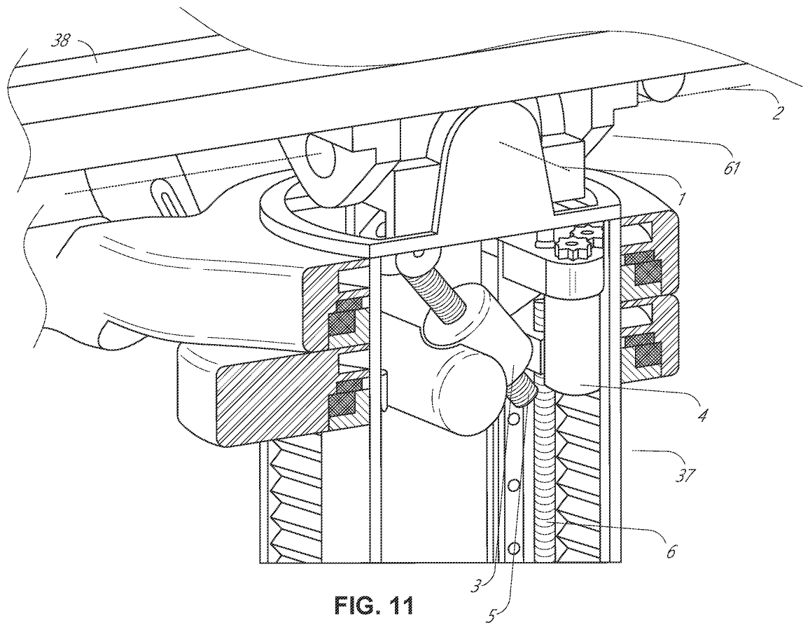

FIG. 11 provides a detailed illustration of the interface between the table 38 and the column 37. Pitch rotation mechanism 61 may be configured to alter the pitch angle of the table 38 relative to the column 37 in multiple degrees of freedom. The pitch rotation mechanism 61 may be enabled by the positioning of orthogonal axes 1, 2 at the column-table interface, each axis actuated by a separate motor 3, 4 responsive to an electrical pitch angle command. Rotation along one screw 5 would enable tilt adjustments in one axis 1, while rotation along the other screw 6 would enable tilt adjustments along the other axis 2. In some embodiments, a ball joint can be used to alter the pitch angle of the table 38 relative to the column 37 in multiple degrees of freedom.

For example, pitch adjustments are particularly useful when trying to position the table in a Trendelenburg position, i.e., position the patient's lower abdomen at a higher position from the floor than the patient's lower abdomen, for lower abdominal surgery. The Trendelenburg position causes the patient's internal organs to slide towards his/her upper abdomen through the force of gravity, clearing out the abdominal cavity for minimally invasive tools to enter and perform lower abdominal surgical or medical procedures, such as laparoscopic prostatectomy.

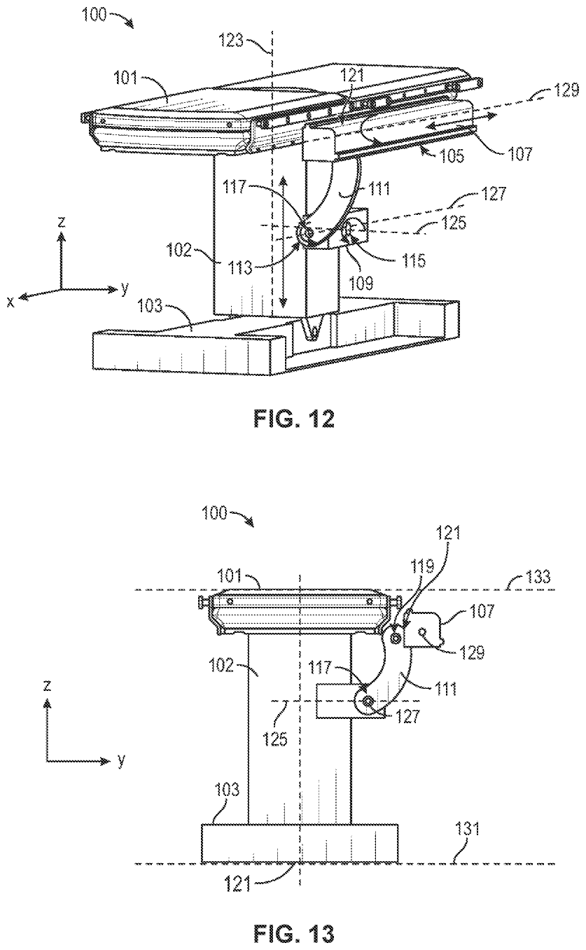

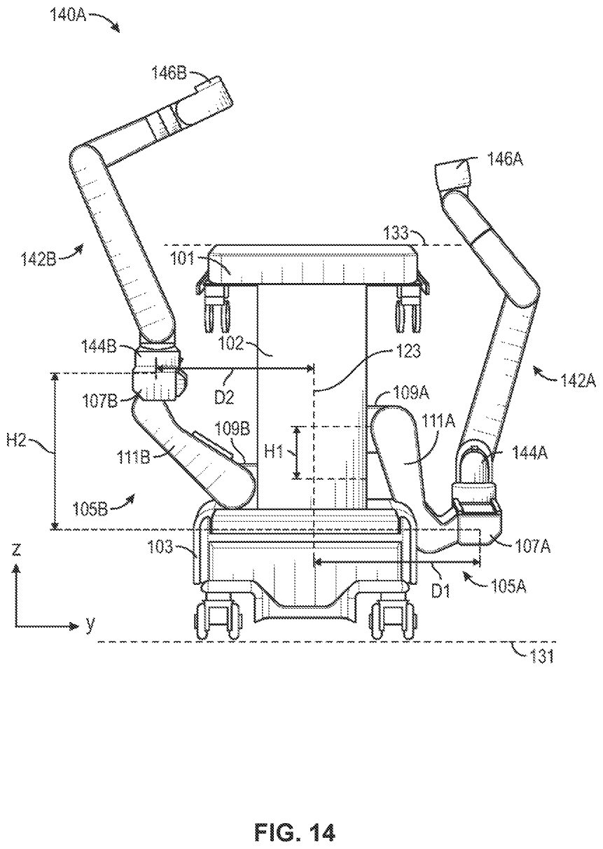

FIGS. 12 and 13 illustrate isometric and end views of an alternative embodiment of a table-based surgical robotics system 100. The surgical robotics system 100 includes one or more adjustable arm supports 105 that can be configured to support one or more robotic arms (see, for example, FIG. 14) relative to a table 101. In the illustrated embodiment, a single adjustable arm support 105 is shown, though an additional arm support can be provided on an opposite side of the table 101. The adjustable arm support 105 can be configured so that it can move relative to the table 101 to adjust and/or vary the position of the adjustable arm support 105 and/or any robotic arms mounted thereto relative to the table 101. For example, the adjustable arm support 105 may be adjusted one or more degrees of freedom relative to the table 101. The adjustable arm support 105 provides high versatility to the system 100, including the ability to easily stow the one or more adjustable arm supports 105 and any robotics arms attached thereto beneath the table 101. The adjustable arm support 105 can be elevated from the stowed position to a position below an upper surface of the table 101. In other embodiments, the adjustable arm support 105 can be elevated from the stowed position to a position above an upper surface of the table 101.

The adjustable arm support 105 can provide several degrees of freedom, including lift, lateral translation, tilt, etc. In the illustrated embodiment of FIGS. 12 and 13, the arm support 105 is configured with four degrees of freedom, which are illustrated with arrows in FIG. 12. A first degree of freedom allows for adjustment of the adjustable arm support 105 in the z-direction (“Z-lift”). For example, the adjustable arm support 105 can include a carriage 109 configured to move up or down along or relative to a column 102 supporting the table 101. A second degree of freedom can allow the adjustable arm support 105 to tilt. For example, the adjustable arm support 105 can include a rotary joint, which can allow the adjustable arm support 105 to be aligned with the bed in a Trendelenburg position. A third degree of freedom can allow the adjustable arm support 105 to “pivot up,” which can be used to adjust a distance between a side of the table 101 and the adjustable arm support 105. A fourth degree of freedom can permit translation of the adjustable arm support 105 along a longitudinal length of the table.

The surgical robotics system 100 in FIGS. 12 and 13 can comprise a table supported by a column 102 that is mounted to a base 103. The base 103 and the column 102 support the table 101 relative to a support surface. A floor axis 131 and a support axis 133 are shown in FIG. 13.

The adjustable arm support 105 can be mounted to the column 102. In other embodiments, the arm support 105 can be mounted to the table 101 or base 103. The adjustable arm support 105 can include a carriage 109, a bar or rail connector 111 and a bar or rail 107. In some embodiments, one or more robotic arms mounted to the rail 107 can translate and move relative to one another.

The carriage 109 can be attached to the column 102 by a first joint 113, which allows the carriage 109 to move relative to the column 102 (e.g., such as up and down a first or vertical axis 123). The first joint 113 can provide the first degree of freedom (“Z-lift”) to the adjustable arm support 105. The adjustable arm support 105 can include a second joint 115, which provides the second degree of freedom (tilt) for the adjustable arm support 105. The adjustable arm support 105 can include a third joint 117, which can provide the third degree of freedom (“pivot up”) for the adjustable arm support 105. An additional joint 119 (shown in FIG. 13) can be provided that mechanically constrains the third joint 117 to maintain an orientation of the rail 107 as the rail connector 111 is rotated about a third axis 127. The adjustable arm support 105 can include a fourth joint 121, which can provide a fourth degree of freedom (translation) for the adjustable arm support 105 along a fourth axis 129.

FIG. 14 illustrates an end view of the surgical robotics system 140A with two adjustable arm supports 105A, 105B mounted on opposite sides of a table 101. A first robotic arm 142A is attached to the bar or rail 107A of the first adjustable arm support 105B. The first robotic arm 142A includes a base 144A attached to the rail 107A. The distal end of the first robotic arm 142A includes an instrument drive mechanism 146A that can attach to one or more robotic medical instruments or tools. Similarly, the second robotic arm 142B includes a base 144B attached to the rail 107B. The distal end of the second robotic arm 142B includes an instrument drive mechanism 146B. The instrument drive mechanism 146B can be configured to attach to one or more robotic medical instruments or tools.

In some embodiments, one or more of the robotic arms 142A, 142B comprises an arm with seven or more degrees of freedom. In some embodiments, one or more of the robotic arms 142A, 142B can include eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and base 144A, 144B (1-degree of freedom including translation). In some embodiments, the insertion degree of freedom can be provided by the robotic arm 142A, 142B, while in other embodiments, the instrument itself provides insertion via an instrument-based insertion architecture.

### C. Instrument Driver & Interface.

The end effectors of the system's robotic arms comprise (i) an instrument driver (alternatively referred to as “instrument drive mechanism” or “instrument device manipulator”) that incorporate electro-mechanical means for actuating the medical instrument and (ii) a removable or detachable medical instrument, which may be devoid of any electro-mechanical components, such as motors. This dichotomy may be driven by the need to sterilize medical instruments used in medical procedures, and the inability to adequately sterilize expensive capital equipment due to their intricate mechanical assemblies and sensitive electronics. Accordingly, the medical instruments may be designed to be detached, removed, and interchanged from the instrument driver (and thus the system) for individual sterilization or disposal by the physician or the physician's staff. In contrast, the instrument drivers need not be changed or sterilized, and may be draped for protection.

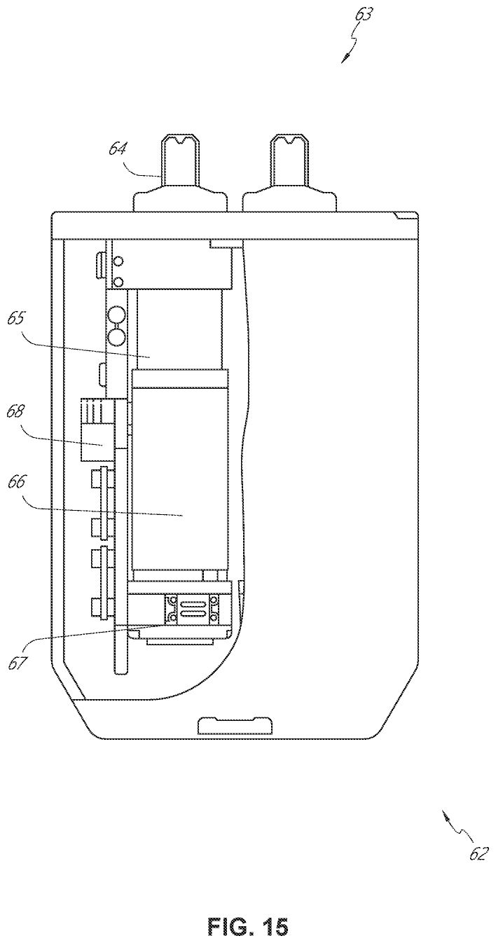

FIG. 15 illustrates an example instrument driver. Positioned at the distal end of a robotic arm, instrument driver 62 comprises of one or more drive units 63 arranged with parallel axes to provide controlled torque to a medical instrument via drive shafts 64. Each drive unit 63 comprises an individual drive shaft 64 for interacting with the instrument, a gear head 65 for converting the motor shaft rotation to a desired torque, a motor 66 for generating the drive torque, an encoder 67 to measure the speed of the motor shaft and provide feedback to the control circuitry, and control circuitry 68 for receiving control signals and actuating the drive unit. Each drive unit 63 being independent controlled and motorized, the instrument driver 62 may provide multiple (four as shown in FIG. 15) independent drive outputs to the medical instrument. In operation, the control circuitry 68 would receive a control signal, transmit a motor signal to the motor 66, compare the resulting motor speed as measured by the encoder 67 with the desired speed, and modulate the motor signal to generate the desired torque.

For procedures that require a sterile environment, the robotic system may incorporate a drive interface, such as a sterile adapter connected to a sterile drape, that sits between the instrument driver and the medical instrument. The chief purpose of the sterile adapter is to transfer angular motion from the drive shafts of the instrument driver to the drive inputs of the instrument while maintaining physical separation, and thus sterility, between the drive shafts and drive inputs. Accordingly, an example sterile adapter may comprise of a series of rotational inputs and outputs intended to be mated with the drive shafts of the instrument driver and drive inputs on the instrument. Connected to the sterile adapter, the sterile drape, comprised of a thin, flexible material such as transparent or translucent plastic, is designed to cover the capital equipment, such as the instrument driver, robotic arm, and cart (in a cart-based system) or table (in a table-based system). Use of the drape would allow the capital equipment to be positioned proximate to the patient while still being located in an area not requiring sterilization (i.e., non-sterile field). On the other side of the sterile drape, the medical instrument may interface with the patient in an area requiring sterilization (i.e., sterile field).

### D. Medical Instrument.

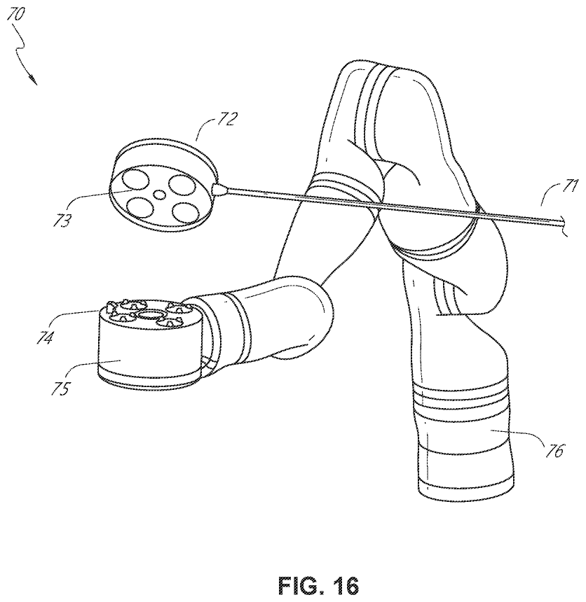

FIG. 16 illustrates an example medical instrument with a paired instrument driver. Like other instruments designed for use with a robotic system, medical instrument 70 comprises an elongated shaft 71 (or elongate body) and an instrument base 72. The instrument base 72, also referred to as an “instrument handle” due to its intended design for manual interaction by the physician, may generally comprise rotatable drive inputs 73, e.g., receptacles, pulleys or spools, that are designed to be mated with drive outputs 74 that extend through a drive interface on instrument driver 75 at the distal end of robotic arm 76. When physically connected, latched, and/or coupled, the mated drive inputs 73 of instrument base 72 may share axes of rotation with the drive outputs 74 in the instrument driver 75 to allow the transfer of torque from drive outputs 74 to drive inputs 73. In some embodiments, the drive outputs 74 may comprise splines that are designed to mate with receptacles on the drive inputs 73.

The elongated shaft 71 is designed to be delivered through either an anatomical opening or lumen, e.g., as in endoscopy, or a minimally invasive incision, e.g., as in laparoscopy. The elongated shaft 71 may be either flexible (e.g., having properties similar to an endoscope) or rigid (e.g., having properties similar to a laparoscope) or contain a customized combination of both flexible and rigid portions. When designed for laparoscopy, the distal end of a rigid elongated shaft may be connected to an end effector extending from a jointed wrist formed from a clevis with at least one degree of freedom and a surgical tool or medical instrument, such as, for example, a grasper or scissors, that may be actuated based on force from the tendons as the drive inputs rotate in response to torque received from the drive outputs 74 of the instrument driver 75. When designed for endoscopy, the distal end of a flexible elongated shaft may include a steerable or controllable bending section that may be articulated and bent based on torque received from the drive outputs 74 of the instrument driver 75.

Torque from the instrument driver 75 is transmitted down the elongated shaft 71 using tendons along the shaft 71. These individual tendons, such as pull wires, may be individually anchored to individual drive inputs 73 within the instrument handle 72. From the handle 72, the tendons are directed down one or more pull lumens along the elongated shaft 71 and anchored at the distal portion of the elongated shaft 71, or in the wrist at the distal portion of the elongated shaft. During a surgical procedure, such as a laparoscopic, endoscopic or hybrid procedure, these tendons may be coupled to a distally mounted end effector, such as a wrist, grasper, or scissor. Under such an arrangement, torque exerted on drive inputs 73 would transfer tension to the tendon, thereby causing the end effector to actuate in some way. In some embodiments, during a surgical procedure, the tendon may cause a joint to rotate about an axis, thereby causing the end effector to move in one direction or another. Alternatively, the tendon may be connected to one or more jaws of a grasper at distal end of the elongated shaft 71, where tension from the tendon cause the grasper to close.

In endoscopy, the tendons may be coupled to a bending or articulating section positioned along the elongated shaft 71 (e.g., at the distal end) via adhesive, control ring, or other mechanical fixation. When fixedly attached to the distal end of a bending section, torque exerted on drive inputs 73 would be transmitted down the tendons, causing the softer, bending section (sometimes referred to as the articulable section or region) to bend or articulate. Along the non-bending sections, it may be advantageous to spiral or helix the individual pull lumens that direct the individual tendons along (or inside) the walls of the endoscope shaft to balance the radial forces that result from tension in the pull wires. The angle of the spiraling and/or spacing there between may be altered or engineered for specific purposes, wherein tighter spiraling exhibits lesser shaft compression under load forces, while lower amounts of spiraling results in greater shaft compression under load forces, but also exhibits limits bending. On the other end of the spectrum, the pull lumens may be directed parallel to the longitudinal axis of the elongated shaft 71 to allow for controlled articulation in the desired bending or articulable sections.

In endoscopy, the elongated shaft 71 houses a number of components to assist with the robotic procedure. The shaft may comprise of a working channel for deploying surgical tools (or medical instruments), irrigation, and/or aspiration to the operative region at the distal end of the shaft 71. The shaft 71 may also accommodate wires and/or optical fibers to transfer signals to/from an optical assembly at the distal tip, which may include of an optical camera. The shaft 71 may also accommodate optical fibers to carry light from proximally-located light sources, such as light emitting diodes, to the distal end of the shaft.

At the distal end of the instrument 70, the distal tip may also comprise the opening of a working channel for delivering tools for diagnostic and/or therapy, irrigation, and aspiration to an operative site. The distal tip may also include a port for a camera, such as a fiberscope or a digital camera, to capture images of an internal anatomical space. Relatedly, the distal tip may also include ports for light sources for illuminating the anatomical space when using the camera.

In the example of FIG. 16, the drive shaft axes, and thus the drive input axes, are orthogonal to the axis of the elongated shaft. This arrangement, however, complicates roll capabilities for the elongated shaft 71. Rolling the elongated shaft 71 along its axis while keeping the drive inputs 73 static results in undesirable tangling of the tendons as they extend off the drive inputs 73 and enter pull lumens within the elongated shaft 71. The resulting entanglement of such tendons may disrupt any control algorithms intended to predict movement of the flexible elongated shaft during an endoscopic procedure.

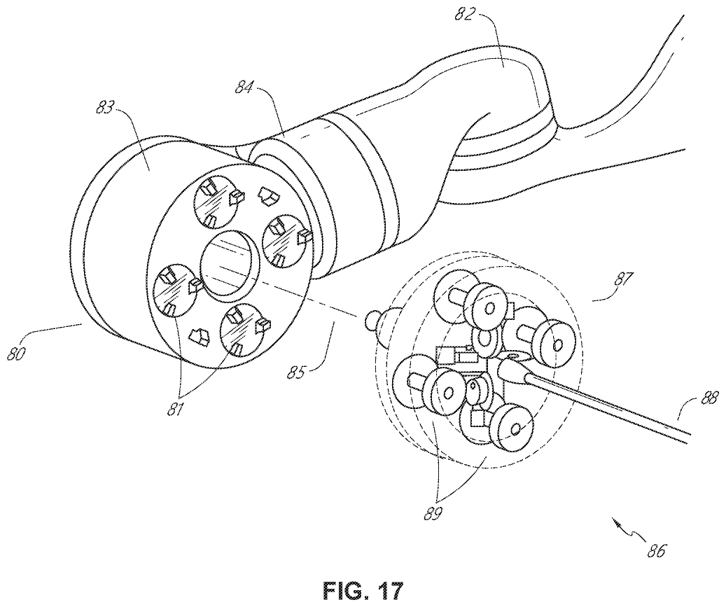

FIG. 17 illustrates an alternative design for an instrument driver and instrument where the axes of the drive units are parallel to the axis of the elongated shaft of the instrument. As shown, a circular instrument driver 80 comprises four drive units with their drive outputs 81 aligned in parallel at the end of a robotic arm 82. The drive units, and their respective drive outputs 81, are housed in a rotational assembly 83 of the instrument driver 80 that is driven by one of the drive units within the assembly 83. In response to torque provided by the rotational drive unit, the rotational assembly 83 rotates along a circular bearing that connects the rotational assembly 83 to the non-rotational portion 84 of the instrument driver. Power and controls signals may be communicated from the non-rotational portion 84 of the instrument driver 80 to the rotational assembly 83 through electrical contacts may be maintained through rotation by a brushed slip ring connection (not shown). In other embodiments, the rotational assembly 83 may be responsive to a separate drive unit that is integrated into the non-rotatable portion 84, and thus not in parallel to the other drive units. The rotational mechanism 83 allows the instrument driver 80 to rotate the drive units, and their respective drive outputs 81, as a single unit around an instrument driver axis 85.

Like earlier disclosed embodiments, an instrument 86 may comprise an elongated shaft portion 88 and an instrument base 87 (shown with a transparent external skin for discussion purposes) comprising a plurality of drive inputs 89 (such as receptacles, pulleys, and spools) that are configured to receive the drive outputs 81 in the instrument driver 80. Unlike prior disclosed embodiments, instrument shaft 88 extends from the center of instrument base 87 with an axis substantially parallel to the axes of the drive inputs 89, rather than orthogonal as in the design of FIG. 16.

When coupled to the rotational assembly 83 of the instrument driver 80, the medical instrument 86, comprising instrument base 87 and instrument shaft 88, rotates in combination with the rotational assembly 83 about the instrument driver axis 85. Since the instrument shaft 88 is positioned at the center of instrument base 87, the instrument shaft 88 is coaxial with instrument driver axis 85 when attached. Thus, rotation of the rotational assembly 83 causes the instrument shaft 88 to rotate about its own longitudinal axis. Moreover, as the instrument base 87 rotates with the instrument shaft 88, any tendons connected to the drive inputs 89 in the instrument base 87 are not tangled during rotation. Accordingly, the parallelism of the axes of the drive outputs 81, drive inputs 89, and instrument shaft 88 allows for the shaft rotation without tangling any control tendons.

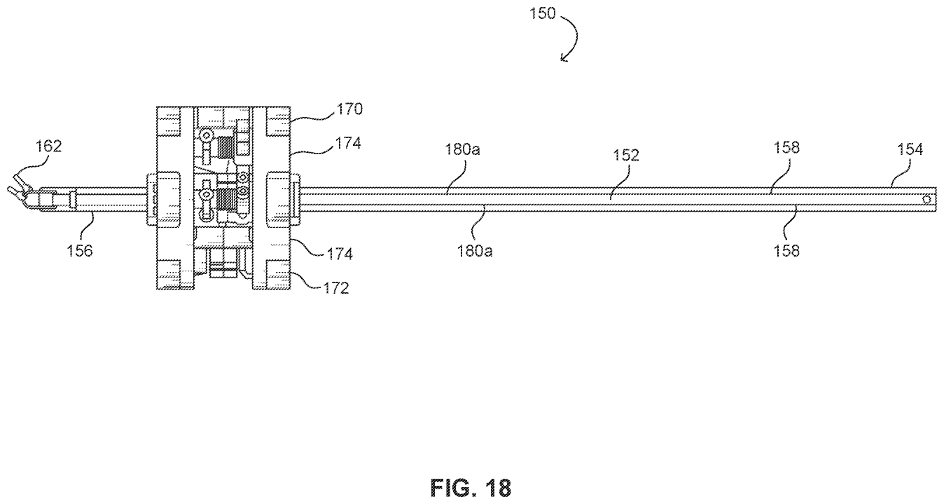

FIG. 18 illustrates an instrument having an instrument based insertion architecture in accordance with some embodiments. The instrument 150 can be coupled to any of the instrument drivers discussed above. The instrument 150 comprises an elongated shaft 152, an end effector 162 connected to the shaft 152, and a handle 170 coupled to the shaft 152. The elongated shaft 152 comprises a tubular member having a proximal portion 154 and a distal portion 156. The elongated shaft 152 comprises one or more channels or grooves 158 along its outer surface. The grooves 158 are configured to receive one or more wires or cables 180 therethrough. One or more cables 180 thus run along an outer surface of the elongated shaft 152. In other embodiments, cables 180 can also run through the elongated shaft 152. Manipulation of the one or more cables 180 (e.g., via an instrument driver) results in actuation of the end effector 162.

The instrument handle 170, which may also be referred to as an instrument base, may generally comprise an attachment interface 172 having one or more mechanical inputs 174, e.g., receptacles, pulleys or spools, that are designed to be reciprocally mated with one or more torque couplers on an attachment surface of an instrument driver.

In some embodiments, the instrument 150 comprises a series of pulleys or cables that enable the elongated shaft 152 to translate relative to the handle 170. In other words, the instrument 150 itself comprises an instrument-based insertion architecture that accommodates insertion of the instrument, thereby minimizing the reliance on a robot arm to provide insertion of the instrument 150. In other embodiments, a robotic arm can be largely responsible for instrument insertion.

### E. Controller.

Any of the robotic systems described herein can include an input device or controller for manipulating an instrument attached to a robotic arm. In some embodiments, the controller can be coupled (e.g., communicatively, electronically, electrically, wirelessly and/or mechanically) with an instrument such that manipulation of the controller causes a corresponding manipulation of the instrument e.g., via primary-secondary control (e.g., primary-follower control).

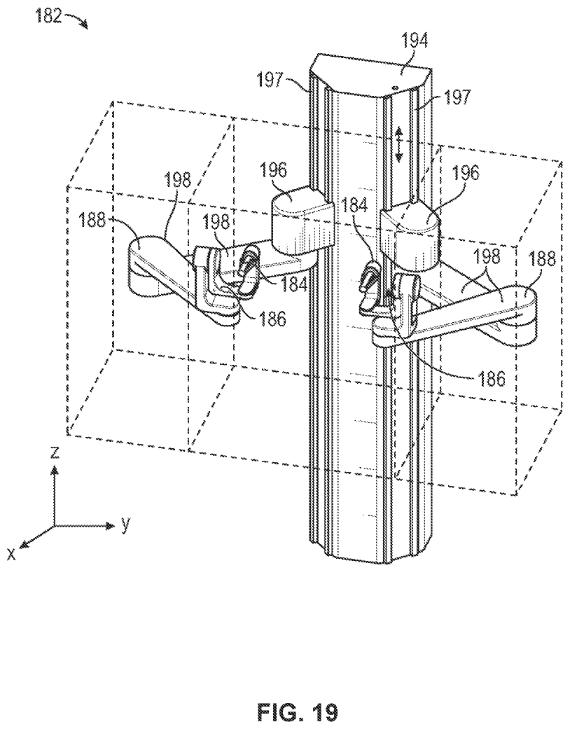

FIG. 19 is a perspective view of an embodiment of a controller 182. In the present embodiment, the controller 182 comprises a hybrid controller that can have both impedance and admittance control. In other embodiments, the controller 182 can utilize just impedance or passive control. In other embodiments, the controller 182 can utilize just admittance control. By being a hybrid controller, the controller 182 advantageously can have a lower perceived inertia while in use.

In the illustrated embodiment, the controller 182 is configured to allow manipulation of two medical instruments, and includes two handles 184. Each of the handles 184 is connected to a gimbal 186. Each gimbal 186 is connected to a positioning platform 188.

As shown in FIG. 19, each positioning platform 188 includes a SCARA arm (selective compliance assembly robot arm) 198 coupled to a column 194 by a prismatic joint 196. The prismatic joints 196 are configured to translate along the column 194 (e.g., along rails 197) to allow each of the handles 184 to be translated in the z-direction, providing a first degree of freedom. The SCARA arm 198 is configured to allow motion of the handle 184 in an x-y plane, providing two additional degrees of freedom.

In some embodiments, one or more load cells are positioned in the controller. For example, in some embodiments, a load cell (not shown) is positioned in the body of each of the gimbals 186. By providing a load cell, portions of the controller 182 are capable of operating under admittance control, thereby advantageously reducing the perceived inertia of the controller while in use. In some embodiments, the positioning platform 188 is configured for admittance control, while the gimbal 186 is configured for impedance control. In other embodiments, the gimbal 186 is configured for admittance control, while the positioning platform 188 is configured for impedance control. Accordingly, for some embodiments, the translational or positional degrees of freedom of the positioning platform 188 can rely on admittance control, while the rotational degrees of freedom of the gimbal 186 rely on impedance control.

### F. Navigation and Control.

Traditional endoscopy may involve the use of fluoroscopy (e.g., as may be delivered through a C-arm) and other forms of radiation-based imaging modalities to provide endoluminal guidance to an operator physician. In contrast, the robotic systems contemplated by this disclosure can provide for non-radiation-based navigational and localization means to reduce physician exposure to radiation and reduce the amount of equipment within the operating room. As used herein, the term “localization” may refer to determining and/or monitoring the position of objects in a reference coordinate system. Technologies such as pre-operative mapping, computer vision, real-time EM tracking, and robot command data may be used individually or in combination to achieve a radiation-free operating environment. In other cases, where radiation-based imaging modalities are still used, the pre-operative mapping, computer vision, real-time EM tracking, and robot command data may be used individually or in combination to improve upon the information obtained solely through radiation-based imaging modalities.

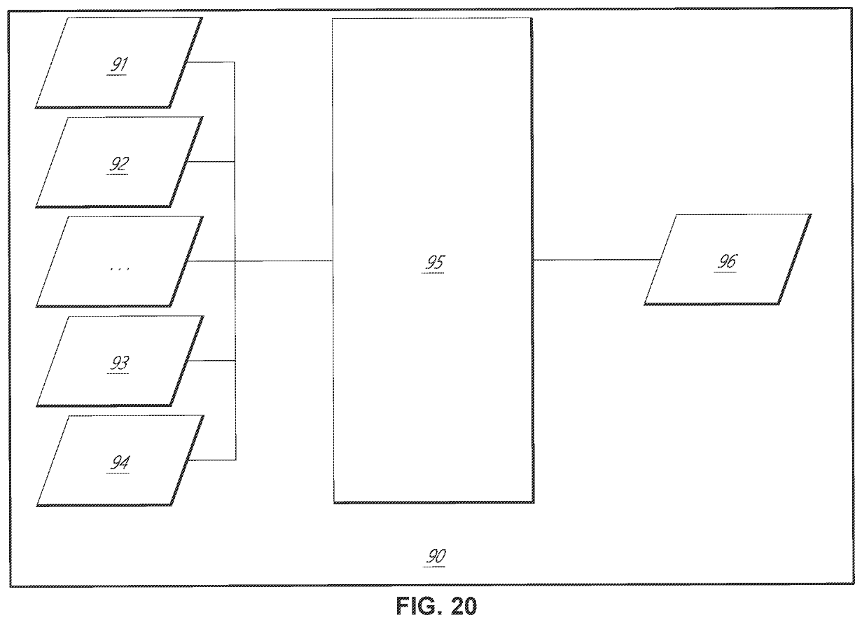

FIG. 20 is a block diagram illustrating a localization system 90 that estimates a location of one or more elements of the robotic system, such as the location of the instrument, in accordance to an example embodiment. The localization system 90 may be a set of one or more computer devices configured to execute one or more instructions. The computer devices may be embodied by a processor (or processors) and computer-readable memory in one or more components discussed above. By way of example and not limitation, the computer devices may be in the tower 30 shown in FIG. 1, the cart shown in FIGS. 1-4, the beds shown in FIGS. 5-14, etc.

As shown in FIG. 20, the localization system 90 may include a localization module 95 that processes input data 91-94 to generate location data 96 for the distal tip of a medical instrument. The location data 96 may be data or logic that represents a location and/or orientation of the distal end of the instrument relative to a frame of reference. The frame of reference can be a frame of reference relative to the anatomy of the patient or to a known object, such as an EM field generator (see discussion below for the EM field generator).

The various input data 91-94 are now described in greater detail. Pre-operative mapping may be accomplished through the use of the collection of low dose CT scans. Pre-operative CT scans are reconstructed into three-dimensional images, which are visualized, e.g. as “slices” of a cutaway view of the patient's internal anatomy. When analyzed in the aggregate, image-based models for anatomical cavities, spaces and structures of the patient's anatomy, such as a patient lung network, may be generated. Techniques such as center-line geometry may be determined and approximated from the CT images to develop a three-dimensional volume of the patient's anatomy, referred to as model data 91 (also referred to as “preoperative model data” when generated using only preoperative CT scans). The use of center-line geometry is discussed in U.S. patent application Ser. No. 14/523,760, the contents of which are herein incorporated in its entirety. Network topological models may also be derived from the CT-images, and are particularly appropriate for bronchoscopy.

In some embodiments, the instrument may be equipped with a camera to provide vision data 92. The localization module 95 may process the vision data to enable one or more vision-based location tracking. For example, the preoperative model data may be used in conjunction with the vision data 92 to enable computer vision-based tracking of the medical instrument (e.g., an endoscope or an instrument advance through a working channel of the endoscope). For example, using the preoperative model data 91, the robotic system may generate a library of expected endoscopic images from the model based on the expected path of travel of the endoscope, each image linked to a location within the model. Intra-operatively, this library may be referenced by the robotic system in order to compare real-time images captured at the camera (e.g., a camera at a distal end of the endoscope) to those in the image library to assist localization.

Other computer vision-based tracking techniques use feature tracking to determine motion of the camera, and thus the endoscope. Some features of the localization module 95 may identify circular geometries in the preoperative model data 91 that correspond to anatomical lumens and track the change of those geometries to determine which anatomical lumen was selected, as well as the relative rotational and/or translational motion of the camera. Use of a topological map may further enhance vision-based algorithms or techniques.

Optical flow, another computer vision-based technique, may analyze the displacement and translation of image pixels in a video sequence in the vision data 92 to infer camera movement. Examples of optical flow techniques may include motion detection, object segmentation calculations, luminance, motion compensated encoding, stereo disparity measurement, etc. Through the comparison of multiple frames over multiple iterations, movement and location of the camera (and thus the endoscope) may be determined.

The localization module 95 may use real-time EM tracking to generate a real-time location of the endoscope in a global coordinate system that may be registered to the patient's anatomy, represented by the preoperative model. In EM tracking, an EM sensor (or tracker) comprising of one or more sensor coils embedded in one or more locations and orientations in a medical instrument (e.g., an endoscopic tool) measures the variation in the EM field created by one or more static EM field generators positioned at a known location. The location information detected by the EM sensors is stored as EM data 93. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. These distances and orientations may be intra-operatively “registered” to the patient anatomy (e.g., the preoperative model) in order to determine the geometric transformation that aligns a single location in the coordinate system with a position in the pre-operative model of the patient's anatomy. Once registered, an embedded EM tracker in one or more positions of the medical instrument (e.g., the distal tip of an endoscope) may provide real-time indications of the progression of the medical instrument through the patient's anatomy.

Robotic command and kinematics data 94 may also be used by the localization module 95 to provide localization data 96 for the robotic system. Device pitch and yaw resulting from articulation commands may be determined during pre-operative calibration. Intra-operatively, these calibration measurements may be used in combination with known insertion depth information to estimate the position of the instrument. Alternatively, these calculations may be analyzed in combination with EM, vision, and/or topological modeling to estimate the position of the medical instrument within the network.

As FIG. 20 shows, a number of other input data can be used by the localization module 95. For example, although not shown in FIG. 20, an instrument utilizing shape-sensing fiber can provide shape data that the localization module 95 can use to determine the location and shape of the instrument.

The localization module 95 may use the input data 91-94 in combination(s). In some cases, such a combination may use a probabilistic approach where the localization module 95 assigns a confidence weight to the location determined from each of the input data 91-94. Thus, where the EM data may not be reliable (as may be the case where there is EM interference) the confidence of the location determined by the EM data 93 can be decrease and the localization module 95 may rely more heavily on the vision data 92 and/or the robotic command and kinematics data 94.

As discussed above, the robotic systems discussed herein may be designed to incorporate a combination of one or more of the technologies above. The robotic system's computer-based control system, based in the tower, bed and/or cart, may store computer program instructions, for example, within a non-transitory computer-readable storage medium such as a persistent magnetic storage drive, solid state drive, or the like, that, upon execution, cause the system to receive and analyze sensor data and user commands, generate control signals throughout the system, and display the navigational and localization data, such as the position of the instrument within the global coordinate system, anatomical map, etc.

### 2. Coordinated Motion of a Patient Platform System

As shown in several of the examples described above, robotic medical systems can include a table that includes a bed or table top. The table can be configured to support a patient during a medical procedure, such as robotic endoscopy, robotic laparoscopy, open procedures, or others (see, for example, FIGS. 1, 3, 4, 5, 8, and 9, described above). In some cases, the table top may need to be moved (translated or rotated) during a medical procedure in order to improve visibility or accessibility to anatomical parts of a patient and thus, coordinated motion of the table top and robotic arms of the robotic medical system can be used to maintain the position of medical tools with respect to a patient during movement of the table top.

Disclosed herein is a patient platform system that advantageously provides coordinated motion between a table top and one or more kinematic chains of the patient platform system. When the patient platform system is in use (e.g., supporting a patient during a medical procedure or transport), a change in a position and/or orientation of the table top can be accompanied by coordinated movement of one or more kinematic chains so that any medical tool(s) attached to the one or more kinematic chains maintain their position relative to the table top.

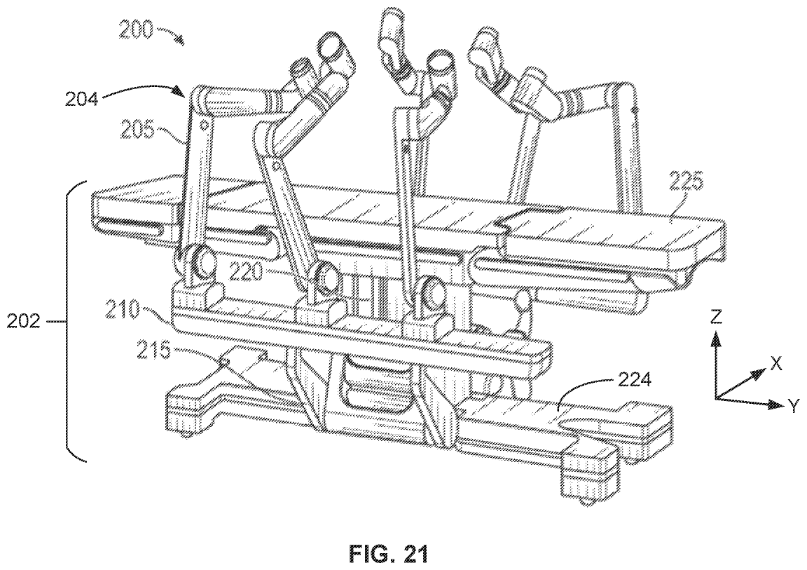

FIG. 21 illustrates a patient platform system 200 in accordance with some embodiments. The patient platform system 200 (e.g., medical platform system, robotic surgical system) includes a table 202 and one or more kinematic chains 204 that are coupled to the table 202. The table 202 includes a rigid base 224 and a table top 225 (e.g., surgical bed, surgical table, robotic surgical table) that is moveable relative to the rigid base 224. The rigid base 224 (e.g., a table base for a surgical bed, a rigid load-bearing housing, a chassis) is configured to support the table top 225 (e.g., with a bed column 220 included in the patient platform system 200). In some embodiments, the table top 225 can be translated, rotated (e.g., yawing), and/or tilted (e.g., pitching and/or rolling) with respect the rigid base 224. The table top 225 serves as a hospital bed or a surgical bed and provides a surface for supporting a patient during a medical procedure or patient transport.

FIG. 21 also illustrates an example x, y, z coordinate system that will be used to describe certain features of the patient platform system 200. It will be appreciated that this coordinate system is provided for purposes of example and explanation only and that other coordinate systems may be used. In the illustrated example, the x-direction or x-axis extends in a lateral direction across the patient platform system 200. For example, the x-direction extends across the table top 225 from one lateral side (e.g., the right side) to the other lateral side (e.g., the left side) when the table top 225 is parallel to the rigid base 224. The y-direction or y-axis extends in a longitudinal direction along the table top 225. For example, the y-direction extends along the table top 225 from one longitudinal end (e.g., the head end) to the other longitudinal end (e.g., the foot end) when the table top 225 is parallel to the rigid base 224. In the illustrated example, the z-direction or z-axis extends along the bed column 220 in a vertical direction (e.g., a direction that is perpendicular to the x-axis and the y-axis).

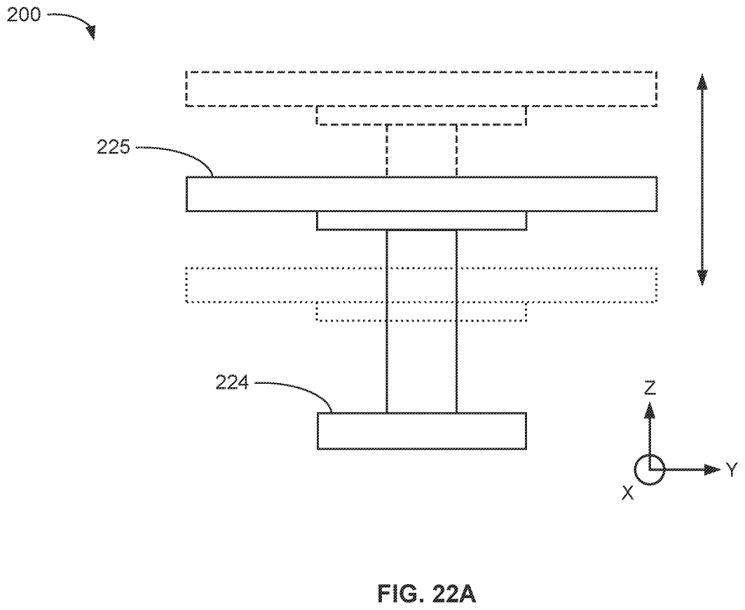

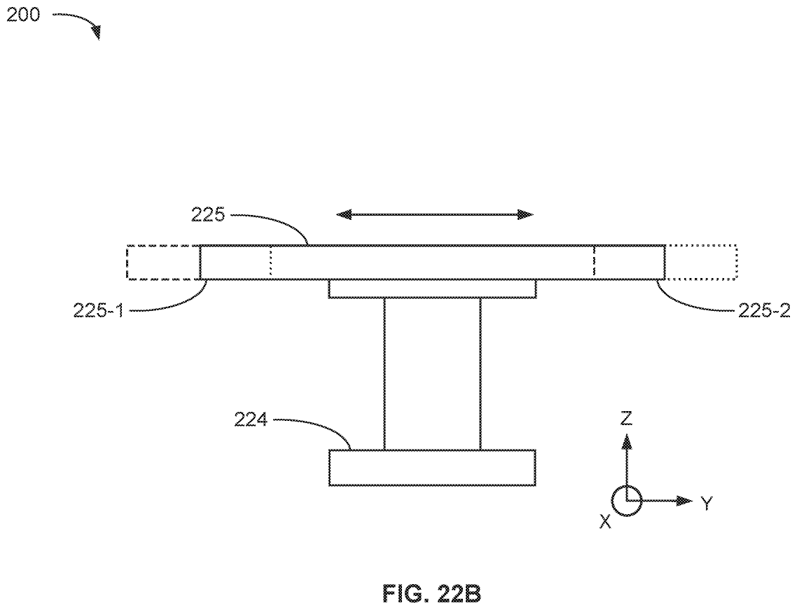

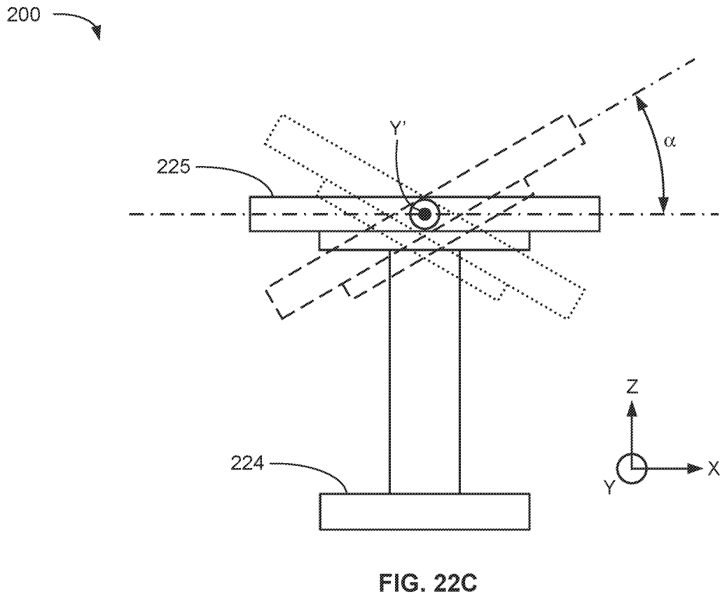

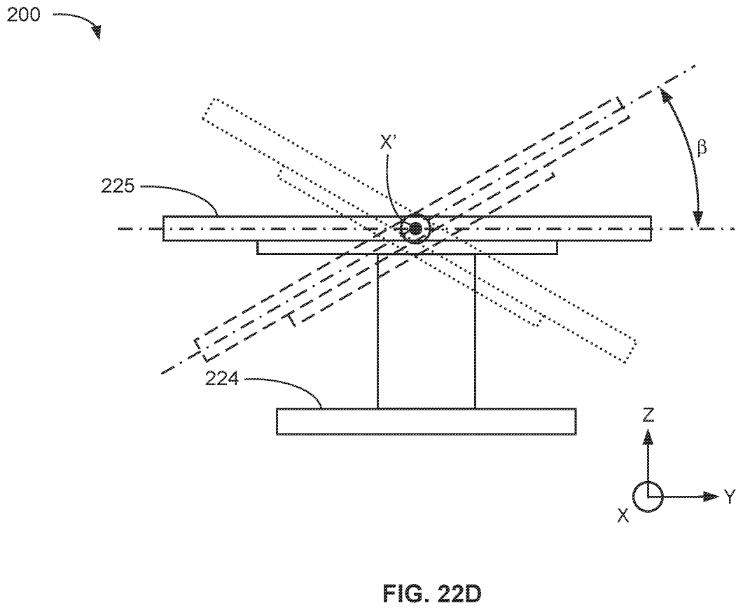

During certain medical procedures, it may be beneficial to change a position or orientation of the table top 225 while the patient is being supported by the table top 225. For example, during a cholecystectomy procedure, a physician may desire to rotate the table top 225 in order to access the patient's gallbladder. In another example, during a hysterectomy procedure, a physician may desire to tilt (e.g., pitch) the table top 225 with respect to a transverse axis (e.g., x-axis) of the table 202 in order to position the table top 225 in a reverse Trendelenburg position (with the patient's feet elevated above the patient's head) in order to access the patient's uterus. As described with reference to FIGS. 22A-22D, the table top 225 can be translated up and down (e.g., along the z-axis), translated longitudinally in the y-direction towards a head or feet of the table top 225 (e.g., along the y-axis), rolled by rotating the table top 225 about a longitudinal axis that is parallel to the y-axis, or pitched by rotating the table top 225 about a transverse axis that is parallel to the x-axis. In some embodiments, the table top 225 can be yawed by rotating the table top 225 about a vertical axis that is parallel to the z-axis.

The one or more kinematic chains 204 include one or more robotic arms 205 and one or more adjustable arm supports 210 configured to support one or more robotic arms 205. For example, the patient platform system 200 shown in FIG. 21 includes two kinematic chains, and each kinematic chain 204 includes an adjustable arm support 210 and three robotic arms 205.

In some embodiments, the patient platform system 200 also includes one or more set-up joints 215. Each of the robotic arms 205 may be supported by one of the adjustable arm supports 210 and the adjustable arm support(s) 210 may be in turn supported by the set-up joint(s) 215. The set-up joint(s) 215 allow movement (e.g., translation and/or rotation) of the adjustable arm support(s) 210 relative to the rigid base 224.

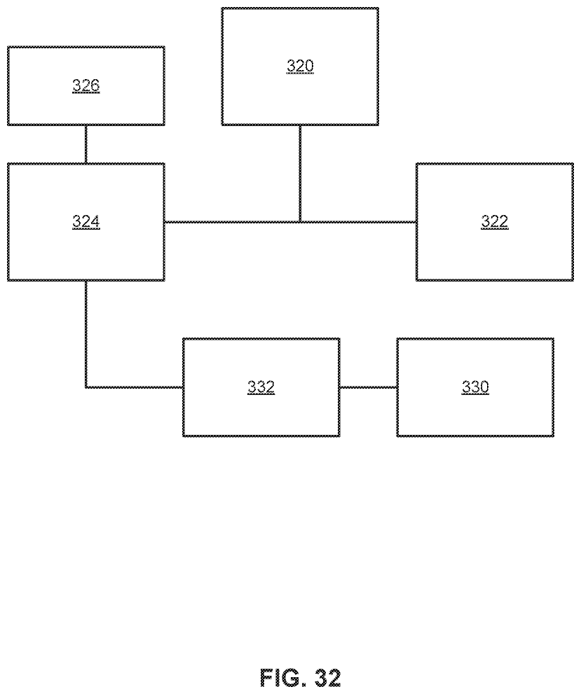

The patient platform system 200 also includes, or is coupled to, one or more processors and memory storing instructions, which, when executed by the one or more processors, cause the processors to operate or move the table top 225 in accordance with a user request and operate or move the one or more robotic arms 205 in accordance with movement of the table top 225. The one or more processors and the memory may be included in the patient platform system 200 or in another system (e.g., a controller or a tower) that is separate from the patient platform system. Electrical connection of the one or more processors and memory is described with respect to FIG. 32. The one or more processors control coordinated movement of any components of the patient platform system 200, such as the table top 225, the bed column 220, the adjustable arm supports 210, and/or the robotic arms 205, as needed in order to achieve coordinated movement of the table top 225 and the kinematic chains in accordance with a user requested movement.

FIG. 22A is a side view of the patient platform system 200, with the x-axis extending into the page, that illustrates axial translation along the z-direction of the table top 225 relative to the rigid base 224. Movement of the table top 225 in the axial direction, represented by the double-ended arrow, allows the table top 225 to be raised or lowered relative to a position of the rigid base 224. The table top 225 is illustrated in three positions: the solid lines represent the table top 225 in an untranslated position, the dashed lines represent the table top 225 in a raised position, and the dotted lines represent the table top 225 in a lowered position.

FIG. 22B is a side view of the patient platform system 200, with the x-axis extending into the page, that illustrates longitudinal translation (along the y-direction) of the table top 225 relative to the rigid base 224. Movement of the table top 225 along the y-direction, as represented by the double-ended arrows, allows the table top 225 to slide towards a head end 225-1 or a feet end 225-2 of the patient platform system 200 relative to the rigid base 224. The table top 225 is illustrated in three positions: the solid lines represent the table top 225 in an untranslated position (e.g., a center of the table top 225 is aligned with a center of the rigid base 224), the dashed lines represent a position where the table top 225 is translated towards the head end 225-1 of the patient platform system 200, the dotted lines represent a position where the table top 225 is translated towards the feet end 225-2 of the patient platform system 200.

FIG. 22C is an end view of the patient platform system 200, with the y-axis extending into the page, that illustrates rolling the table top 225 around a longitudinal axis Y′ that is parallel to the y-axis (e.g., extends in the y-direction) of the patient platform system 200. This allows the table top 225 to be rolled or rotated left to right. The table top 225 is illustrated in three positions: the solid lines representing in an un-rotated position where the table top 225 is substantially parallel to the rigid base 224, the dashed lines representing a first rotated position, and the dotted lines representing a second rotated position. In the first rotated position, the table top 225 forms an angle α with respect to the x-axis, and in the second rotated position, the table top 225 forms a negative angle α with respect to the x-axis. In some embodiments, the table top 225 can be configured to allow at least a rotational angle α of about 5 degrees, 10 degrees, 15 degrees, 20 degrees, 25 degrees, 30 degrees, 35 degrees, 40 degrees, 45 degrees or more relative to the un-rotated position. In some embodiments, the table top 225 can be rotated in both transverse directions from the un-rotated position (e.g., the rotational angle α can be positive or negative). In some embodiments, the table top 225 can be rotated to any angle between the positions illustrated in dashed and dotted lines.

FIG. 22D is a side view of the patient platform system 200, with the x-axis extending into the page, that illustrates pitching the table top 225 around a transverse axis X′ that is parallel to the x-axis (e.g., extends in the x-direction) of the patient platform system 200. This allows the table top 225 to be pitched or tilted such the head of the table top 225 is elevated relative to the feet of the table top 225, or vice versa. The table top 225 is illustrated in three positions: the solid lines representing an un-tilted position where the table top 225 is substantially parallel to the rigid base 224, the dashed lines representing a first tilted position, and the dotted lines representing a second tilted position. In the first tilted position, the table top 225 forms an angle β with respect to the y-axis, and in the second tilted position, the table top 225 forms a negative angle β with respect to the y-axis. In some embodiments, the table top 225 can be configured to allow at least a tilt angle β of about 15 degrees, 20 degrees, 25 degrees, 30 degrees, 35 degrees, 40 degrees, 45 degrees, 50 degrees, 55 degrees, 60 degrees or more relative to the un-tilted position. In some embodiments, the table top 225 can be tilted in both longitudinal directions from the un-tilted position (e.g., the lateral tilt angle β can be positive or negative). In some embodiments, the table top 225 can be tilted to any angle between the positions illustrated in dashed and dotted lines.

Additional details regarding mechanisms associated with movement of the table top 225 are described in application U.S. patent application Ser. No. 16/810,469, filed Mar. 5, 2020, which is incorporated by reference herein in its entirety.

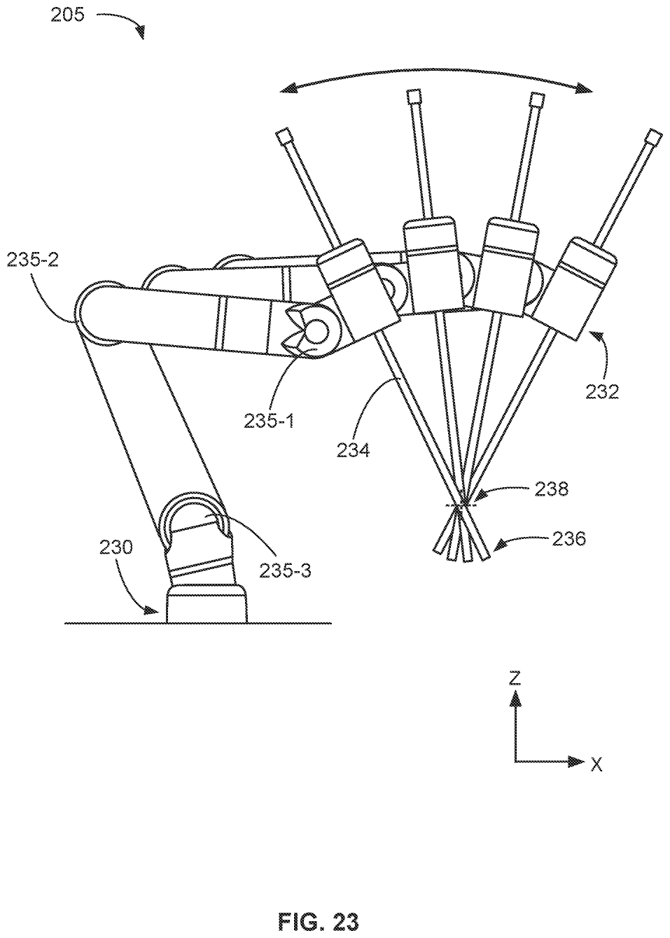

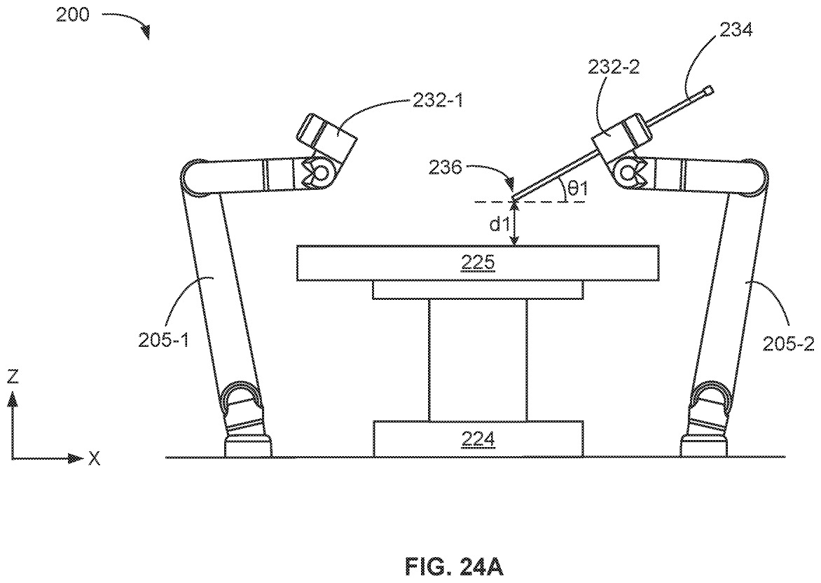

The patient platform system 200 is configured to move the table top 225 in any of the translational and rotational motions described with respect to FIGS. 22A-22D. Two or more of the above-described motions can be performed consecutively or simultaneously. Additionally, in combination with the various translational and rotational movements that can be performed by the table top 225, the one or more kinematic chains 204 of the patient platform system 200 are capable of both independent movement (e.g., manipulating a medical tool around a reference point during a medical procedure) and coordinated movement (e.g., moving the reference point along with the movement of the table top 225 during a medical procedure) with respect to movement of the table top 225. Details regarding operation of the robotic arm 205 are described with respect to FIG. 23.

FIG. 23 illustrates a robotic arm 205 of the patient platform system 200 of FIG. 21 in accordance with some embodiments. The robotic arm 205 includes a first end 230 that is coupled to the table 202, optionally via an adjustable arm support 210 and/or a set-up joint 215, and an active device manipulator (ADM) 232 including one or more drive mechanisms that is configurable to allow a medical tool 234 (e.g., a diagnostic or imaging device, a scope, a surgical instrument) to be attached to the robotic arm 205. The robotic arm 205 includes one or more joints that provide the robotic arm 205 with multiple degrees of freedom and allow the robotic arm 205 to perform various movements. For example, the robotic arm 205 shown in FIG. 23 includes a distal joint 235-1, an intermediate joint 235-2, and a base joint 235-3. In some embodiments, the robotic arm 205 includes one or more kinematically redundant joints to provide additional degrees of freedom.

In particular, one or more joints 235-1, 235-2, 235-3 can control an orientation of the ADM 232 (e.g., a distal portion) of the robotic arm 205. The robotic arm 205 is configurable to move independently of other components of the patient platform system 200, thereby allowing the robotic arm 205 to independently control and manipulate a medical tool 234 attached to the ADM 232 of the robotic arm 205. While movement of the robotic arm 205 can be controlled independently from movements of other components of the patient platform system 200, movement of the robotic arm 205 can also be coordinated with movements of other components of the patient platform system 200, such as movement of the table top 225. A distal end or a distal portion of a kinematic chain may correspond to any of: an ADM 232, a tool tip 236 of a medical tool 234 (while attached to the kinematic chain via the ADM 232 of a robotic arm 205), or a remote center of motion (RCM) 238 of the robotic arm 205.

In some embodiments, movement of a kinematic chain, including a robotic arm 205, can be coordinated with lateral and/or rotational movement of the table top 225 such that one or more preset conditions are maintained prior to initiating movement of the kinematic chain 204 and/or the table top 225, while any of the kinematic chain 204 and the table top 225 is in motion, and/or after movement of any of the kinematic chain 204 and the table top 225. In some embodiments, the one or more preset conditions include a constraint or requirement that a position and/or orientation of a tool tip 236 of a medical tool 234 attached to the kinematic chain (e.g., attached to a robotic arm 205 of the kinematic chain) is maintained relative to a position and orientation of the table top 225. In some embodiments, the one or more preset conditions include a requirement that the position of the remote center of motion 238 of a robotic arm 205 is maintained relative to the table top 225. In some embodiments, a remote center of motion 238 of the robotic arm 205 and the tool tip position of the medical tool 234 attached to the robotic arm 205 coincide with one another (e.g., the remote center of motion 238 correspond to the tool tip position of the medical tool 234). In some embodiments, the one or more preset conditions limit movement of the distal end (e.g., ADM 232, tool tip 236 of a medical tool 234 attached to the robotic arm 205) of the kinematic chain 204 to less than a threshold amount of movement relative to the position of the table top 225.

In some embodiments, the one or more preset conditions prohibit any portion of the kinematic chain, such as a portion of the robotic arm 205, and the table top 225 from reaching within a threshold distance of each other during movement (e.g., motion) of any of the kinematic chain 204 and the table top 225. For example, if a user requests rotation of the table top 225 to a position in which the table top 225 would collide with any portion of a robotic arm 205, the patient platform system 200 would either prohibit the movement or move the robotic arm 205 (if possible without violating any other preset conditions) out of the way of a path of the table top 225 in order to prevent collision or contact between the table top 225 and the robotic arm 205. In some embodiments, the one or more preset conditions prohibit a first robotic arm and a second robotic arm, of the one or more robotic arms coupled to the same table 202, from reaching within a threshold distance of one another during movement (e.g., motion) of any of the first robotic arm and the second robotic arm. For example, if a user requests movement of the table top 225 to a position in which coordinated movement of the kinematic chains would result in a first and a second robotic arm (that are each coupled to the same table 202) colliding or coming into contact with each other in order to maintain any of the other preset conditions (e.g., maintaining a position of the tool tip 236 of the medical tool 234 relative to the table top 225), the patient platform system 200 would either prohibit movement of the table top 225 or move at least one of the first or second robotic arms (if possible without violating any other preset conditions) in order to prevent collision or contact between the first and second robotic arms.

In some embodiments, the one or more preset conditions prevent the robotic arm 205 from reaching within a threshold distance (e.g., a fixed distance, a dynamically variable threshold distance based on a potential well) of one or more objects adjacent to the patient platform system 200 (e.g., to avoid collision or impact between the patient platform system 200 and the patient or medical personnel nearby). In some embodiments, to maintain the preset condition, one or more of the robotic arms 205 are moved out of the way to avoid a robotic arm 205 from getting too close (e.g., reaching within a threshold distance) to a location of a physical object, such as the patient, medical personnel, or additional medical equipment, such as a vital signs monitoring device, pumps, etc. In some embodiments, the location of the patient on the table top 225 is specified by a restricted zone relative to movement of the table top 225, and the robotic arms 205 are automatically moved out of the restricted zone by the one or more processors during the first movement of the table top 225, and/or prevented from entering the restricted zone during the coordinated movement executed by the robotic arms 205 in accordance with the first movement of the table top 225. In some embodiments, undocked robotic arms are moved (e.g., through translation and/or rotations of various joints along the robotic arms relative to the base or arm support, and/or translation and/or rotation of the adjustable arm support coupled to the base) to avoid regions of the physical environment where medical personnel is likely to be present and/or where unexpected impact with the patient platform system 200 had previously occurred in a prior movement. In some embodiments, in order to maintain the preset condition, the configuration of one or more of the robotic arms 205 (e.g., while the base joints of the robotic arms 205 are kept stationary relative to the adjustable arm support, and/or during translation along the adjustable arm support) are changed to prevent the robotic arms 205 from getting too close (e.g., reaching within a threshold distance) to restricted zones and/or regions with high probability of impact.

In some embodiments, the one or more preset conditions require that a fixed spatial relationship (e.g., fixed position and/or orientation) between the medical tool 234 (e.g., a scope, a surgical instrument, in a retracted state or an un-retracted state) and the table top 225 is maintained during movement of the table top 225. In some embodiments, portions of the first robotic arm 205 are moved relative to the table top 225 in a null-space operation to maintain the relative position and/or relative orientation between the attached medical tool 234 and the table top 225. In a null-space operation, a robotic arm 205 includes at least one redundant joint that enables it to assume various positions and orientations for a state of the robotic arm. For example, in some embodiments, it may be desirable to maintain a remote center of motion associated with the robotic arm, while moving the table top. The robotic arm can maintain a certain state (e.g., its remote center of motion), while its links and joints move in various configurations in the null-space.

In some embodiments, the one or more preset conditions require that an aligned spatial relationship between the table top 225 and a tele-operation input device of the medical tool 234 attached to the robotic arm 205 is maintained during movement of the table top 225.

In some embodiments, the one or more preset conditions permit spatial relationships between the table top 225 and a distal portion of the one or more kinematic chain to change by more than a threshold amount during movement of the table top 225. For example, if the robotic arm 205 of a kinematic chain is not deployed (e.g., not in use, not in a docked state, is not attached to a medical tool 234, is in a retracted state, is attached to a medical tool 234 that is in a retracted state), the relative position of the distal end of the kinematic chain (e.g., an ADM 232 of the robotic arm 205 or a medical tool 234 attached to the robotic arm 205) to the table top 225 may be allowed to change during movement of the table top 225. In this case, since the robotic arm 205 and any medical tool 234 attached to the robotic arm 205 are not involved in a medical procedure and therefore not in use or in contact with a patient, a change in the relative position of the distal end of the kinematic chain (e.g., change in the relative position of the ADM 232 of the robotic arm 205, change in the relative position of any medical tool 234 attached to the robotic arm 205) to the table top 225 does not have an adverse effect on the medical procedure or the patient. Additionally, in some cases, the undocked robotic arm 205 may be moved to compensate for a shift in a center of gravity of the patient during movement of the table top 225, alleviate pressure due to contact of the patient with a portion of the robotic arm 205, or prevent contact of the robotic arm 205 with the patient during movement of the table top 225.

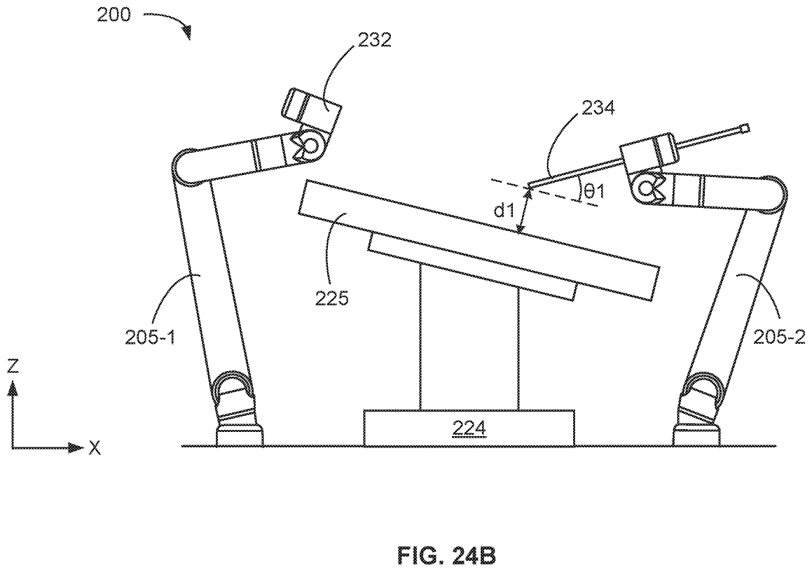

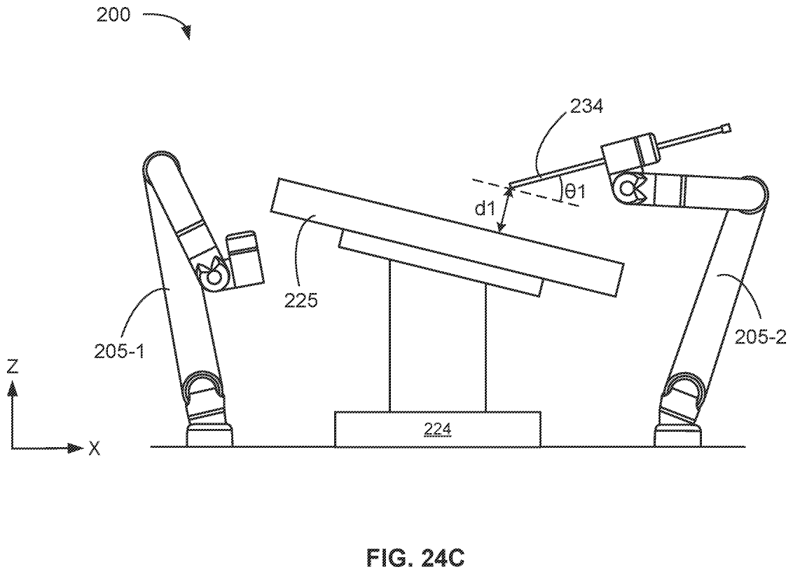

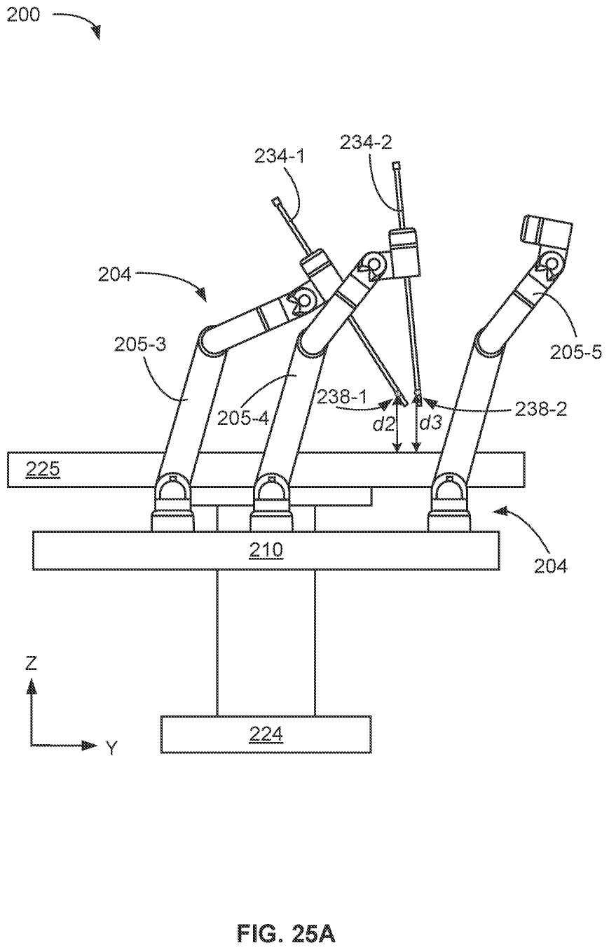

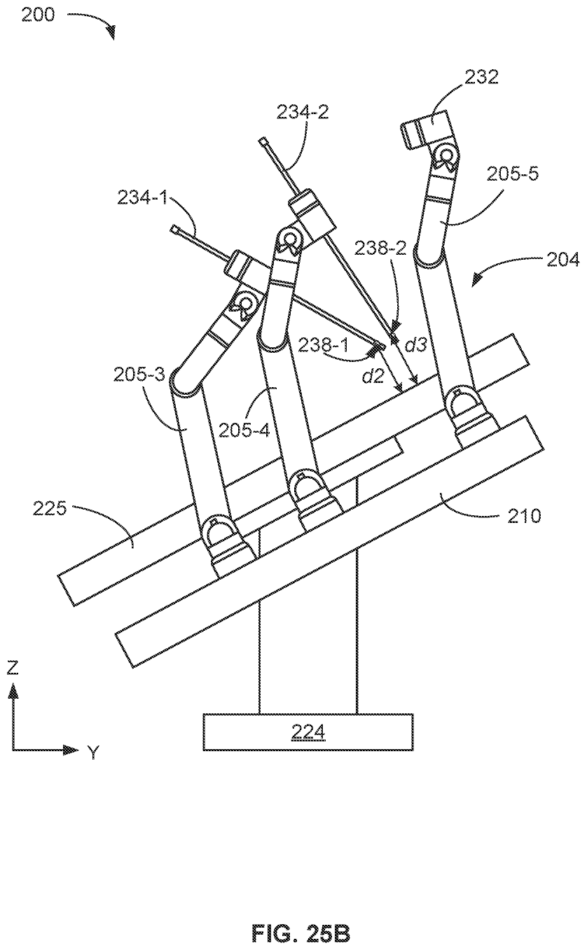

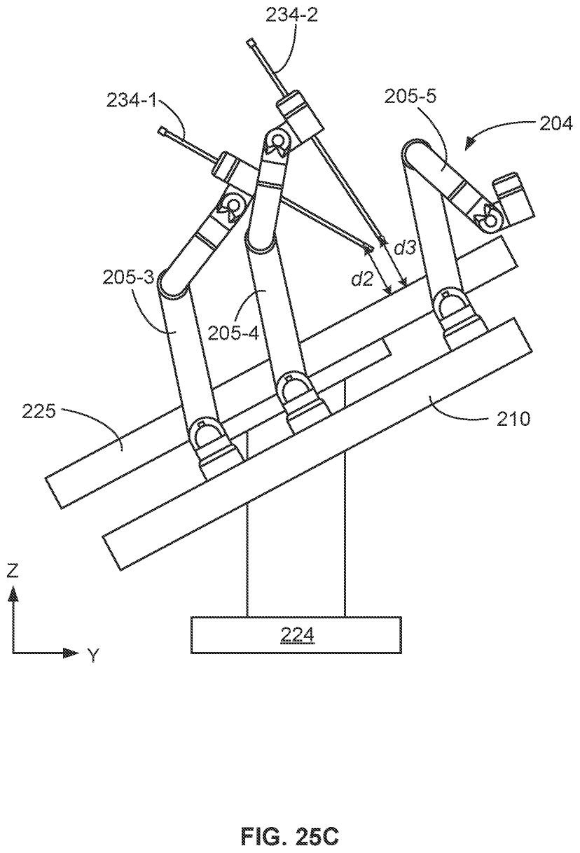

Two examples of coordinated motion between the table top 225 and robotic arms 205 of the patient platform system 200 are described with respect to FIGS. 24A-24C and FIGS. 25A-25C.

FIGS. 24A-24C illustrate end views of the patient platform system 200 and show coordinated motion of two robotic arms 205-1 and 205-2 with the table top 225 of the patient platform system 200 in accordance with some embodiments. FIG. 24A shows the table top 225 in a starting position where the table top 225 is substantially parallel to the rigid base 224, the robotic arm 205-1 in an undocked state (e.g., not-in-use state), and the robotic arm 205-2 is in a docked state (e.g., an in-use state, a deployed state) with a medical tool 234 attached to an ADM 232 of the robotic arm 205-2. The tool tip 236 of the medical tool 234 has a relative position and orientation with respect to the table top 225. In particular, the tool tip 236 of the medical tool 234 is located at a distance d1 from a surface of the table top 225 and is oriented at an angle θ1 with respect to a plane, represented by a dashed line, that is parallel with a plane of the table top 225. While movement of the table top 225 and both the robotic arms 205-1 and 205-2 must meet one or more preset condition(s), the robotic arm 205-1 is subject to a first set of the preset condition(s) that apply to robotic arms that are in an undocked state. In contrast, the robotic arm 205-2 is subject to a second set of the preset condition(s) that apply to robotic arms that are in a docked state. The first set of preset conditions is different from the second set of preset conditions. Some of the preset condition(s), such as a condition that prohibits movement that will result in collision (e.g., contact) between two robotic arms and/or collision between a robotic arm 205 and the table top 225, are applicable to all robotic arms regardless of their state (e.g., applicable to both docked and undocked robotic arms), and such preset conditions are part of both the first set and the second set of the preset conditions.

FIG. 24B shows the table top 225 in a tilted position where the table top 225 is rotated (e.g., rolled) on an x-z plane (e.g., rotated around a longitudinal axis parallel to the y-axis). FIG. 24B may represent the patient platform system 200 during motion of the table top 225 or represent a final state where a requested or desired position of the table top 225 is achieved. Since the robotic arm 205-2 is in a docked state, the robotic arm 205-2 is moved in coordination with movement of the table top 225 such that the one or more preset conditions are met. In this example, the one or more preset conditions include maintaining a relative position and orientation of the tool tip 236 of a medical tool 234 attached to the robotic arm 205-2 with respect to the table top 225. Thus, the robotic arm 205-2 is moved in coordination with movement of the table top 225 such that the position and orientation of the of the tool tip 236 of the medical tool 234 attached to the robotic arm 205-2 is maintained during movement of the table top 225. In contrast to the robotic arm 205-2, the robotic arm 205-1 is in an undocked or not-in-use position and thus, the robotic arm 205-1 can be moved without restriction or limitation regarding a distal end position of the robotic arm 205-1. In some embodiments, the robotic arm 205-1 is not moved in response to movement of the table top 225 and movement of the robotic arm 205-2. In this example, the initial position of the robotic arm 205-1 complies with the one or more preset conditions (e.g., collision avoidance) while the table top 225 and the robotic arm 205-2 are moved. Thus, in some embodiments, the robotic arm 205-1 can remain stationary during movement of the table top 225 and the robotic arm 205-2. In other embodiments, as shown in FIG. 24B, the robotic arm 205-1 is moved in response to movement of the table top 225 and movement of the robotic arm 205-2 to avoid collision while the table top 225 and the robotic arm 205-2 are moved.

In some embodiments, as shown in FIG. 24C, it may be desirable to move the robotic arm 205-1 even if the robotic arm 205-1 can remain stationary during movement of the table top 225 and the robotic arm 205-2. In this example, robotic arm 205-1 is moved into a retracted position. It may be desirable to move the robotic arm 205-1, for example, to reduce any potential interference with a medical procedure being performed or in order to redistribute weight across the patient platform system 200 thereby reducing stress and strain (and wear and tear) of components of the patient platform system 200. Additionally, while the one or more preset conditions may be met during movement of the table top 225 and movement of the robotic arm 205-2, a medical attendant may require the robotic arm 205-1 to be moved in order to access an anatomical part of the patient. The robotic arm 205-1 may be manually moved by a user of the patient platform system 200 (e.g., when the robotic arm 205-1 is in a low-impedance mode) or be automatically moved in accordance with user input at an input device configured to provide user commands (e.g., commands or instructions corresponding to user requested movement and/or user requested position) to the patient platform system 200.

While two robotic arms 205-1 and 205-2 are shown in FIGS. 24A-24C, the patient platform system 200 may include additional robotic arms 205 (e.g., three, four, five, six, or more robotic arms in total) and medical tools 234 that are not included in FIGS. 24A-24C. In some configurations, the patient platform system 200 includes only one robotic arm 205. While coordinated movement of the robotic arms 205 may involve movement in a subset (e.g., less than all) of the robotic arms 205 of the patient platform system 200, in some embodiments, all of the robotic arms 205 of the patient platform system 200 comply with the one or more preset conditions during movement of the table top 225. It is also possible, in some embodiments, that all robotic arms 205 of the patient platform system 200 are moved during coordinated motion of the robotic arms 205 with movement of the table top 225.

FIGS. 25A-25C illustrate side views of the patient platform system 200 and show coordinated motion between a kinematic chain 204 and the table top 225 of the patient platform system 200 in accordance with some embodiments. In FIG. 25A, the kinematic chain 204 includes an adjustable arm support 210 and three robotic arms 205-3, 205-4, and 205-5. FIG. 25A also shows that the table top 225 is in an initial position where the table top 225 is substantially parallel to the rigid base 224. The robotic arm 205-3 is in a docked state (e.g., an in-use state, a deployed state) with a medical tool 234-1 attached to an ADM of the robotic arm 205-3, the robotic arm 205-4 is in a docked state (e.g., an in-use state, a deployed state) with a medical tool 234-2 attached to an ADM of the robotic arm 205-4, and the robotic arm 205-5 in an undocked state (e.g., not-in-use state). The respective remote centers of motion 238-1 and 238-2 of the robotic arm 205-3 and 205-4 have respective relative positions with respect to the table top 225. In particular, the remote center of motion 238-1 of the robotic arm 205-3 is located at a distance d2 from a surface of the table top 225 and the remote center of motion 238-2 of the robotic arm 205-4 is located at a distance d3 from the surface of the table top 225. While movement of the table top 225 and movement of any of the three robotic arms 205-3, 205-4, and 205-5 must meet one or more preset condition(s), the robotic arms 205-3 and 205-4 are subject to a first set of the preset condition(s) that apply to robotic arms that are in a docked state. In contrast, the robotic arm 205-5 is subject to a second set of the preset condition(s) that apply to robotic arms that are in an undocked state, where the second set of the preset conditions is different from the first set of the preset conditions. Some of the preset condition(s), such as a condition that prohibits movement that will result in collision (e.g., contact) between robotic arms, between a robotic arm and the table top 225, and/or between a robotic arm and the patient, are applicable to all robotic arms regardless of their state (e.g., applicable to both docked and undocked robotic arms).

FIG. 25B shows the table top 225 in a tilted position where the table top 225 is rotated (e.g., pitched) with respect to the y-axis (e.g., rotated around a transverse axis parallel to the x-axis). The kinematic chain 204 is moved in coordination with movement of the table top 225 such that the one or more preset conditions are met. In this example, the one or more preset conditions include maintaining a relative position of remote center of motion 238 of the docked robotic arms 205 with respect to the table top 225. Movement of the kinematic chain 204 includes movement of the adjustable arm support 210 as well as movement of the robotic arms 205-3 and 205-4. The adjustable arm support 210 has been tilted in coordination with tilting of the table top 225, and the robotic arms 205-3 and 205-4 are moved in coordination with movement of the table top 225 such that the relative positions of the remote centers of motion 238-1 and 238-2 of the robotic arms 205-3 and 205-4, respectively, are maintained during movement of the table top 225. In contrast, the robotic arm 205-5 is in a undocked or not-in-use position and thus, the robotic arm 205-5 can be moved without restriction or limitation regarding a position of the remote center of motion 238 or an ADM 232 of the robotic arm 205-5. In some embodiments, as shown in FIG. 25B, the robotic arm 205-5 is not moved (relative to the adjustable arm support 210) in response to movement of the table top 225 and the robotic arms 205-3 and 205-4.

In some embodiments, as shown in FIG. 25C, the robotic arm 205-5 may need to be moved in order to meet the one or more preset conditions. For example, during movement of the table top 225, a patient supported by the table top 225 may have shifted in position, leading to the patient making contact with the robotic arm 205-5 such that a force is exerted upon the robotic arm 205-5 due to the weight and position of the patient (or a portion of the patient such as a patient's arm or leg). In such cases, the robotic arm 205-5 may be moved to alleviate or reduce the amount of force exerted upon the robotic arm 205-5 by the patient. In this example, the robotic arm 205-5 is moved into a retracted state in order to avoid or alleviate contact with the patient. In some embodiments, the robotic arm 205-5 is moved in response to detecting, by one or more sensors located on the robotic arm 205-5, one or more forces exerted on the robotic arm 205-5. Similarly, the shifting of the patient position may exert on the medical tool 234-1 and/or the medical tool 234-2 a force, which may be detected by one or more sensors associated with the robotic arms 205-3 and 205-4, and the robotic arms 205-3 and/or 205-4 may be moved to alleviate or reduce the amount of force exerted upon the medical tools 234-1 and/or 234-2 by the patient. During such movement of the robotic arms 205-3 and/or 205-4, the robotic arms 205-3 and 205-4 substantially maintain respective remote centers of motion (e.g., the medical tools 234-1 and 234-2 may deviate from the respective remote centers of motion by a distance less than a threshold distance).

While single kinematic chain 204 (including adjustable arm support 210 and robotic arms 205-3, 205-4, and 205-5) is shown in FIGS. 25A-25C, the patient platform system 200 may include additional kinematic chain(s), including additional adjustable arm supports 210, additional robotic arms 205, and additional medical tools 234 that are not illustrated in FIGS. 25A-25C. Each robotic arm 205 of the patient platform system 200 may be engaged in coordinated motion in response to movement of the table top 225 such that all of the robotic arms 205 of the patient platform system 200 comply with the one or more preset conditions during movement of the table top 225.

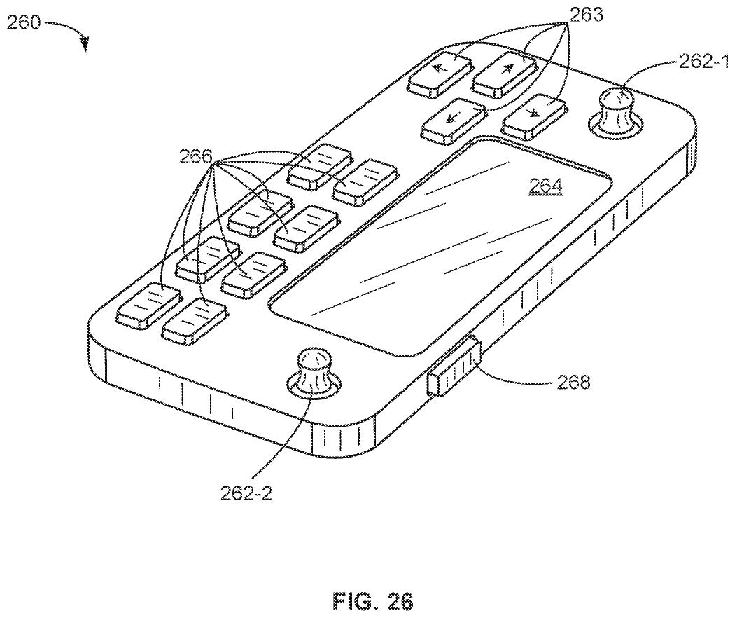

Movement of the patient platform system 200, including movement of the table top 225 can be controlled by a user or operator of the patient platform system 200 via one or more input devices. Addition movement of the kinematic chains 204 may also be controlled via input device(s). FIG. 26 illustrates an input device 260 for receiving user request for movement of the patient platform system 200 of FIG. 21 in accordance with some embodiments.

The input device 260 may communicate with the patient platform system 200 via a wireless connection, such as Bluetooth or over a wireless network, or via one or more wired electrical connections. Thus, the input device 260 may be implemented in different ways—the input device 260 may be a handheld pendant, a controller, a joystick controller, a computer, or even a device with a touch screen surface, such as a tablet or a smart phone. For example, the input device 260 may be located or mounted onto the patient platform system 200 and the input device 260 may be electrically connected to mechanisms configured for moving the table top 225 and mechanisms for moving the one or more robotic arms 205. In another example, the input device may be a smart phone or a tablet that is in communication with the patient platform system 200 via a wireless network or a Bluetooth connection. The smart phone or tablet may include an application that is configured to allow a user to control movement of the patient platform system 200 via user inputs at a touch screen or affordance of the smart phone or tablet. In some embodiments, the input device 260 is able to communicate with the patient platform system 200 within a predefined range of operation (e.g., the patient platform system 200 and the input device are within 5 feet, 10 feet, 20 feet, 50 feet of one another). In yet another example, the input device 260 may be a computer that is configured to run a computer application that allows the user to control movements of the table top 225, and optionally, movements of the one or more robotic arms 205.

In some embodiments, as shown in FIG. 26, the input device 260 includes one or more joysticks 262 and/or one or more directional affordances 263 configured to control translation (e.g., along the z- and y-directions) and rotation (e.g., roll, pitch, or yaw) of the table top. For example, a z-position, y-position, pitch (e.g., rotation around a transverse axis parallel to the x-axis), a roll (e.g., rotation around a longitudinal axis parallel to the y-axis), and a yaw (e.g., rotation around a vertical axis parallel to the z-axis) of the table top 225 can be controlled via one or more user inputs provided using any combination of the joysticks 262 and/or the directional affordances 263. User input can be received simultaneously at multiple directional affordances 263 and/or joysticks 262 in order to achieve a desired motion or a targeted position of the table top 225. In some embodiments, the joysticks 262-1 and 262-2 may be implemented via a touch screen or a touch pad or replaced by additional directional affordances (e.g., affordances corresponding to left, right, forward, and backward directions).

In some embodiments, the input device 260 includes a display 264. The display 264 may present a representation of the table top 225, the one or more kinematic chains 204 (e.g., robotic arms 205 and/or adjustable arm supports 210), and/or additional information regarding the table top 225, such as any warning or error messages, for example, a collision warning or a prohibited motion warning. The input device 260 optionally includes one or more additional affordances 266. An affordance of the one or more additional affordances 266 may correspond to predefined settings of the patient platform system 200, such as preset table top position where the table top 225 is moved to (e.g., a Trendelenburg position or a reverse Trendelenburg position).

In some embodiments, the input device 260 includes a motion affordance 268 and the patient platform system 200 may require that the motion affordance 268 be activated (e.g., pressed) to initiate movement of the table top 225 and/or that the motion affordance 268 be continuously maintained (e.g., continuously pressed) during movement of the table top 225 and coordinated movement of the robotic arms 205. The motion affordance 268 serves as a safety precaution to prevent unintentional movement of the table top 225.

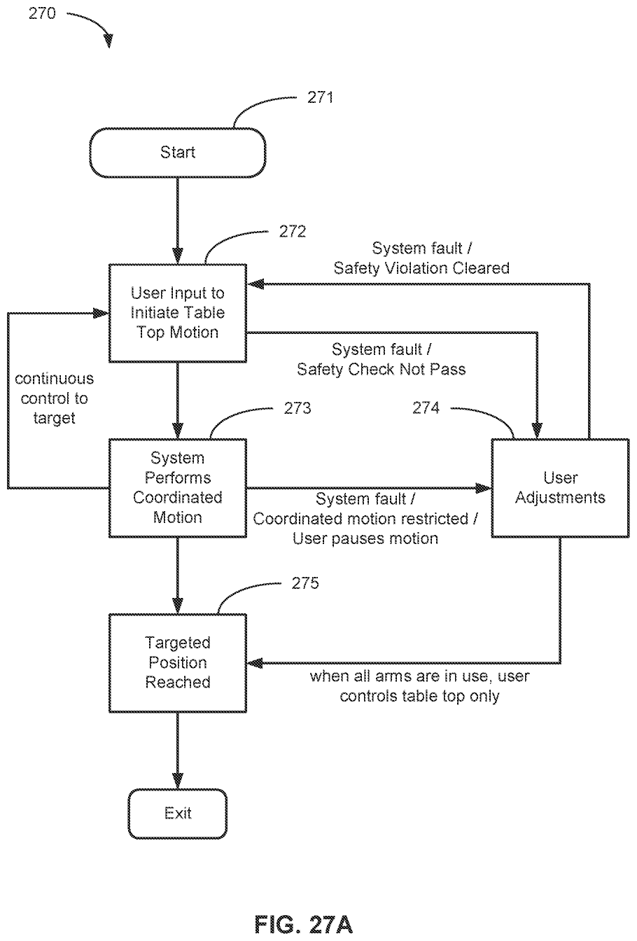

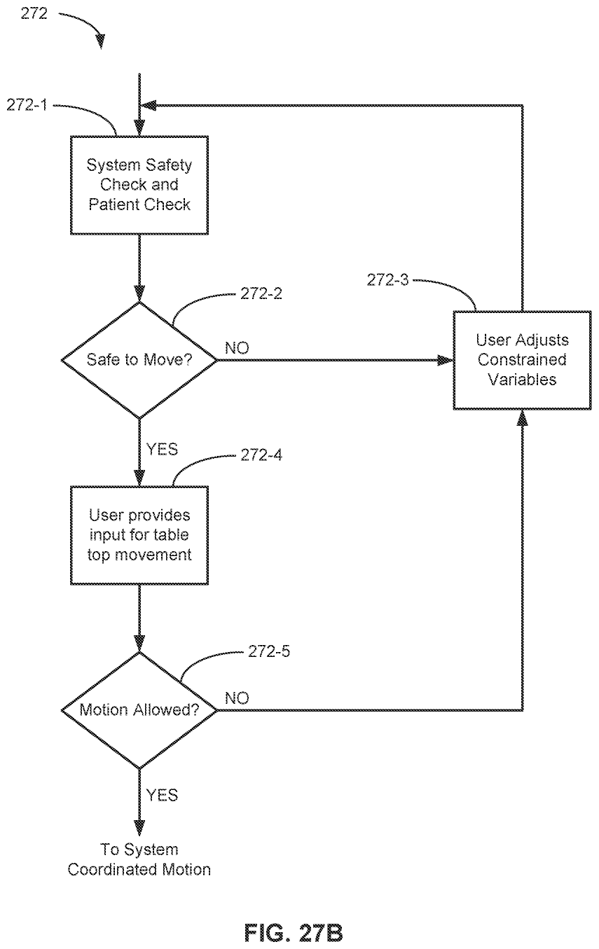

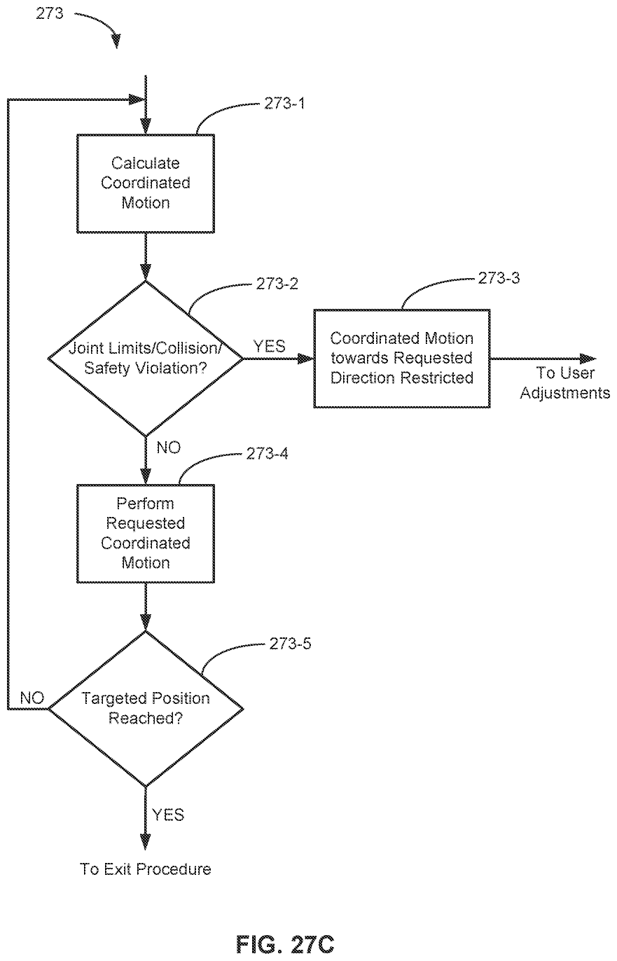

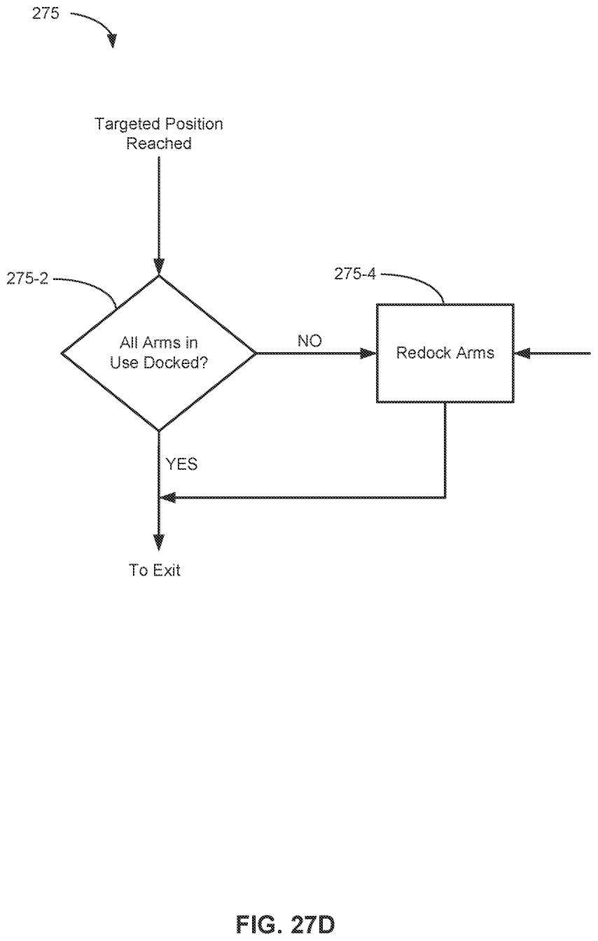

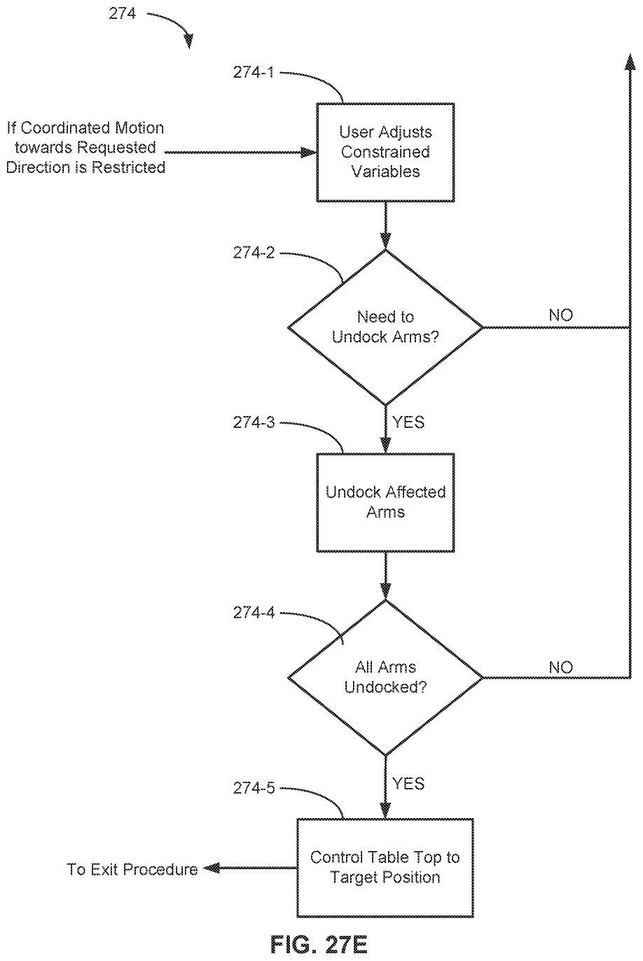

FIGS. 27A-27E show a flowchart 270 for operating the patient platform system 200 in accordance with some embodiments. In FIG. 27A, movement of the table top 225 of the patient platform system 200 begins (271) when a user request to initiate movement of the table top 225 is received (272) by the patient platform system 200. In response to receiving the user request, a system check is performed in order to confirm that all of the applicable preset conditions for movement of the table top 225 are and/or will be met prior to initiating movement of the table top 225 and/or movement of the one or more kinematic chains 204. In accordance with a determination that all preset conditions are and/or will be met (e.g., any safety faults or violations of preset conditions being cleared or resolved, either by the patient platform system 200 or by the user), the patient platform system 200 proceeds to move (273) the table top 225 in accordance with the user input and coordinate movement of the one or more kinematic chains 204 in accordance with movement of the table top 225. When one or more preset conditions for coordinated motion of the table top 225 are not met, either prior to initiating table top movement or during table top movement and/or coordinated movement of the one or more kinematic chains 204, the table top movement and coordinated movement of the one or more kinematic chains 204 are halted until the violations can be resolved and the preset conditions are met via one or more user adjustments (274). Once the patient platform system 200 is in compliance with the preset conditions, movement of the table top 225 and coordinated movement of the one or more robotic arms 205 can be resumed. This process is repeated until a targeted final position of the table top 225 is reached (275).

FIG. 27B illustrates details of the system check performed in connection with initiating coordinated movement. The user performs (272-1) a safety check and confirms (272-2) that all safety requirements for movement of the table top 225 are met. For example, the user may check the position of a patient, including the position of the patient's extremities to make sure that they are secure and in a safe position, and that the patient is safely secured (e.g., fastened) to the table top 225. The user may also check port tension on the ADMs 232 of robotic arms 205, check that cables are undamaged and in a safe location, check that there are no potential obstacles in a pathway of the table top 225 and any potential pathways of the one or more kinematic chains 204, check that there are no potential obstacles in a restricted zone determined in accordance with the requested movement, check that any medical tools 234 attached to the one or more robotic arms 205 are in view of the user and not in contact with the patient (e.g., not in contact with patient tissue), and check that any instruments that are not in use are removed from the vicinity of the patient platform system 200. In the case where at least one of the safety requirements or preset conditions are not met, the user takes action (272-3) to meet all of the safety requirements and the preset conditions before movement of the table top 225 and/or kinematic chains 204 may begin. For example, the user may move one or more robotic arms 205 (in a null-space mode or secondary/follower clutch mode) that are in contact with a patient away from the patient, manually untangle cables and clear the area around the patient platform system 200 of any obstacles, and/or clear any existing system faults. Some of the adjustments performed by the user, such as movement of the one or more robotic arms 205 in a null-space mode, are assisted by functionalities of the patient platform system 200. Additional details regarding null-space motion of robotic arms 205 are described in U.S. patent application Ser. No. 17/010,586, filed Sep. 2, 2020, which is incorporated by reference herein in its entirety.

After checking that all the safety requirements are met, the user provides (272-4) one or more user requests for movement of the table top 225 via one or more user devices (such as user device 260). The patient platform system 200 performs (272-5) one or more checks to confirm that the user requested movement meets the preset conditions and is allowed. For example, the patient platform system 200 may check that the system is not in a fault mode, that all medical tools 234 attached to a robotic arm 205 are in view and under active master control, check a tool tip position of a medical tool 234 if the medical tool 234 is in contact with the patient, check that adjustable arm supports 210, robotic arms 205, and the table top 225 are not in contact with one another, check that the requested motion does not exceed joint limits of joints 235 of a robotic arm 205 (and joint/movement limits of other movable components, such as the adjustable arm supports 210, the set-up joints 215, the bed column 220, the table top 225, etc.), and check that the cannula port tension of a cannula coupled to the robotic arm 205 does not exceed a predetermined threshold tension. In response to a confirmation that the user requested movement meets the preset conditions and is allowed, the patient platform system 200 proceeds to perform coordinated movement of the table top 225 and the one or more kinematic chains 204.

FIG. 27C illustrates details regarding performing (273) coordinated movement of the patient platform system 200. The patient platform system 200 calculates (273-1) coordinated motion of the one or more kinematic chains 204 in accordance with the user requested movement of the table top 225. Coordinated movement includes movement of the table top 225 in accordance with the user request, and movement of one or more kinematic chains 204, such as movement of one or more robotic arms 205 and/or one or more adjustable arm supports 210. The patient platform system 200 calculates coordinated movement of the patient platform system 200, including using inverse kinematics to determine position(s) of any of the table top 225, the robotic arms 205, and the adjustable arm support 210.

Once the coordinated motion of the patient platform system 200 is calculated, the patient platform system 200 determines (273-2) whether or not the calculated motion violates any of the preset conditions. For example, the patient platform system 200 may check to see if the calculated motion will result in collision or contact between any components of the patient platform system 200, such as contact between the table top 225 and a robotic arm 205 or collision between two or more robotic arms 205. The patient platform system 200 may also check to see if any of the calculated movement exceeds joint limits of the joints 235 of the one or more robotic arms 205 (and joint/movement limits of other movable components, such as the adjustable arm supports 210, the set-up joints 215, the bed column 220, the table top 225, etc.), and/or if any of the calculated movement results in a change in the remote center of motion 238 of a docked robotic arm 205 by more than a predetermined movement amount (e.g., more than 5 mm, 10 mm, 15 mm, 20 mm, 25 mm). The patient platform system 200 may also check to see if any of the calculated movement may cause the cannula port tension exerted on an ADM 232 of a robotic arm 205 to exceed the predetermined threshold tension, if any objects are in contact with any of the robotic arms 205, and confirm that the calculated movement does not violate any primary-secondary (e.g., primary-follower) movement conditions or thresholds. In accordance with a determination, by the patient platform system 200, that the calculated movement would violate at least one of the preset conditions, the patient platform system 200 restricts (273-3) movement of the table top 225 and the requested movement is not executed or is halted (in the case where movement has already begun). In such cases, the user may be able to make one or more manual adjustments (274) to clear the path of the calculated movement so that the calculated movement no longer violates the preset conditions. Details and examples of user adjustments (274) are provided with respect to FIG. 27E.

In accordance with a determination, by the patient platform system 200, that the calculated movement complies with (e.g., does not violate) the preset conditions, the patient platform system 200 sends out motion commands for execution by mechanisms of the patient platform system 200 (e.g., drivers) that are responsible for movement of the table top 225 and/or movement of the adjustable arm support 210 and the robotic arms 205 (as applicable). The patient platform system 200 performs (273-4) the coordinated motion in accordance with the commands. During execution of the coordinated motion, the patient platform system 200 continuously monitors a status of the different components (e.g., table top 225, robotic arms 205, adjustable arm supports 210) of the patient platform system 200 to ensure that the preset conditions are met during the entire coordinated movement of kinematic chains 204 with movement of the table top 225. In some embodiments, the user may provide a manual override to pause the coordinated motion by providing a “pause motion” input (such as activating or pressing a pause affordance, like a stop foot pedal or a halt button) via a user input device (such as user input device 260) or by releasing a motion affordance on the user input device (e.g., the motion affordance 268 shown in FIG. 26). For example, the user may pause coordinated motion of the patient platform system 200 due to one or more medical tools 234 moving out of view of a camera, or if the user notices an obstruction in the path of the calculated movement that has gone undetected by the patient platform system 200, such as an arm of a nearby medical personnel. The patient platform system 200 continues to execute (273-5) the calculated coordinated motion of the one or more kinematic chains 204 and the table top 225 until the patient platform system 200 determines that the targeted position, as requested by the user, has been achieved. Once the targeted position of the table top 225 has been reached (275), the patient platform system 200 performs a final check process, shown in FIG. 27D, prior to exiting operation of the coordinated motion of the patient platform system 200.

Referring to FIG. 27D, in accordance with the patient platform system 200 achieving the calculated motion such that the targeted position of the table top 225 has been reached, the user checks (275-2) that all robotic arms 205 that are in use (e.g., required for the medical procedure) are in a docked or deployed position. In the case where one or more robotic arms 205 that are required for the medical procedure are not in the docked position, the user may deploy (275-4) the robotic arms (either manually or through robotic control of the robotic arm 205). In order to re-dock any arms that have been retracted for the coordinated motion, the one or more robotic arms may enter a low-impedance mode that allows the user to position the robotic arms 205 with ease, either manually or through robotic control. In accordance with a determination that any robotic arms 205 required for the medical procedure are in a docked or deployed state, the patient platform system 200 exits the coordinated motion procedure and coordinated motion of the patient platform system 200 is considered to be completed.

As discussed above with respect to FIG. 27C, in some embodiments, one or more user adjustments (274) may be required during coordinated motion of the patient platform system 200. Referring to FIG. 27E, in the case where coordinated motion of the patient platform system 200 is restricted (273-3), the user may adjust (274-1) one or more constrained variables of the patient platform system 200, such as any of: checking robotic arm joints 235 that are near joint limits, checking robotic arms 205 that are in collision with a portion of the patient platform system 200 (including self-collision) or within a predetermined threshold distance from the patient platform system 200, relaxing constraints on ADMs 232 and/or cannulas coupled thereto that are at or near the tension threshold, and removing obstacles that may be in contact with a moveable portion of the patient platform system 200 (such as equipment that is in contact with a robotic arm 205). The patient platform system 200 then makes a determination (274-2) as to whether or not undocking (e.g., retracting) a robotic arm 205 that is involved in violation of the preset condition should be selected (e.g., the undocking is the only possible solution). If undocking one or more robotic arms 205 is selected, the identified robotic arm(s) 205 are undocked (274-3) as necessary. Otherwise, user adjustment is conducted until the violation(s) are resolved and the preset conditions are met.

The patient platform system 200 also determines (274-4) if all of the robotic arms 205 of the patient platform system 200 are undocked. If not, the user can perform additional adjustments to the docked robotic arm(s) 205 as needed to clear the violation. In accordance with a determination that all of the robotic arms 205 of the patient platform system 200 are undocked, the patient platform system 200 allows the user to control (274-5) movement of the table top 225 via user input device(s) while the robotic arms 205 remain in an undocked position. In this case, the patient platform system 200 moves the table top 225 to the targeted position in accordance with the user requested movement. Once the table top 225 reaches (275) the targeted position, the patient platform system 200 performs final checks, as described with respect to FIG. 27D, before exiting the table top motion procedure.

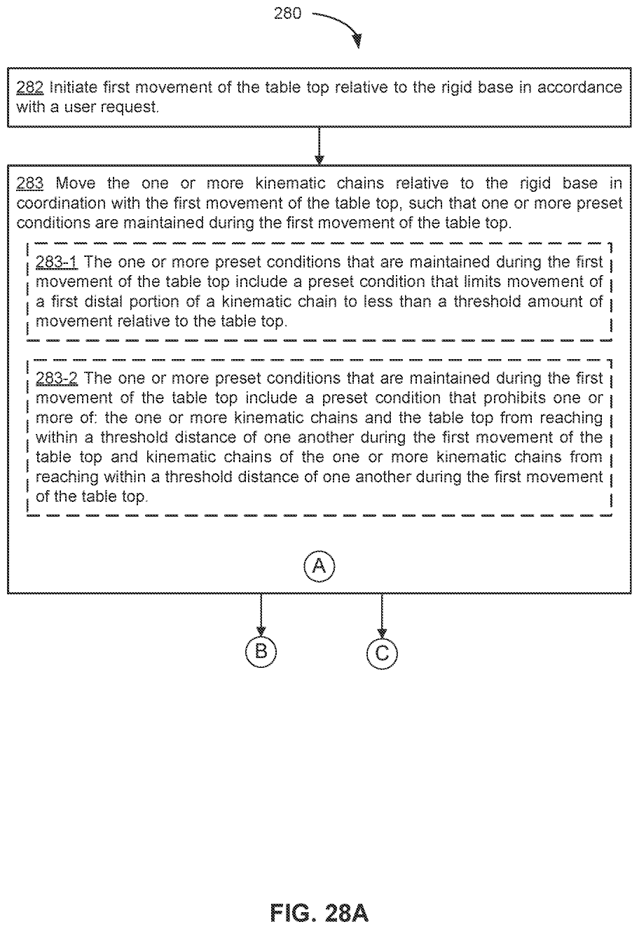

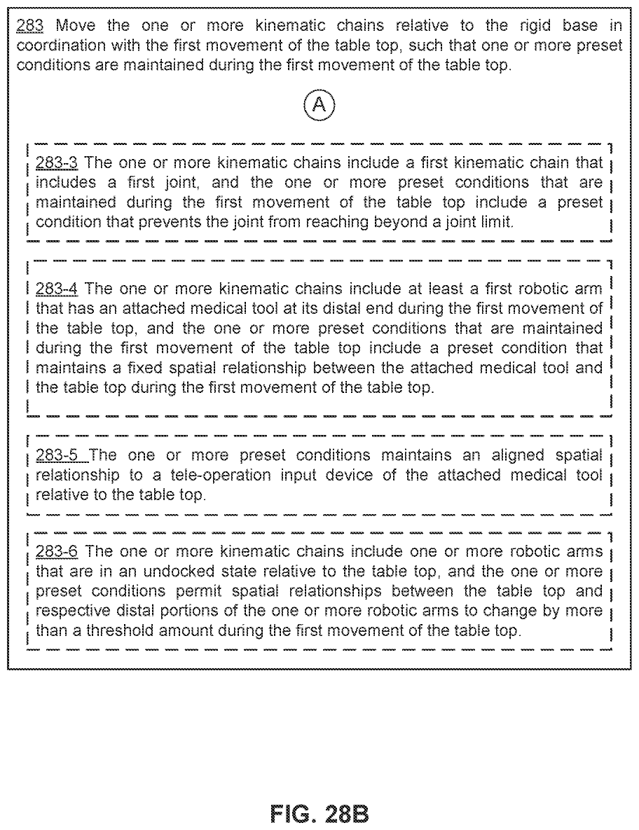

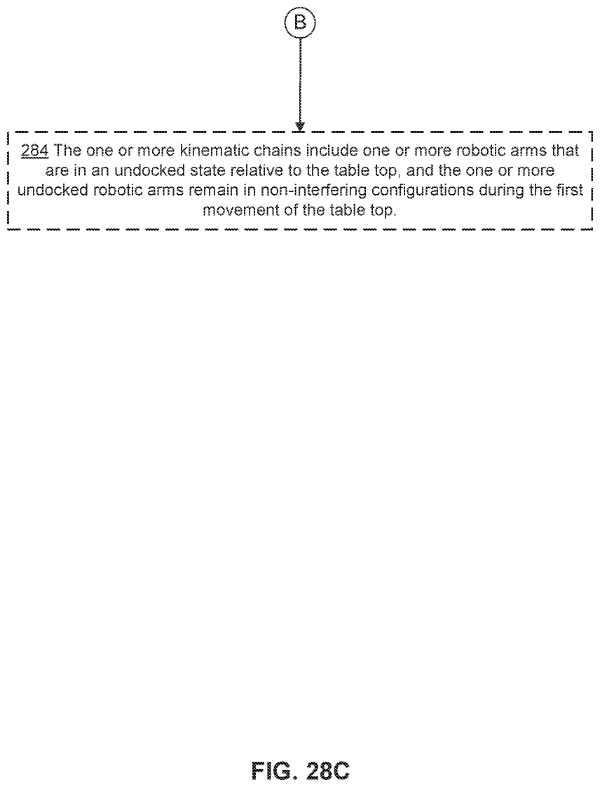

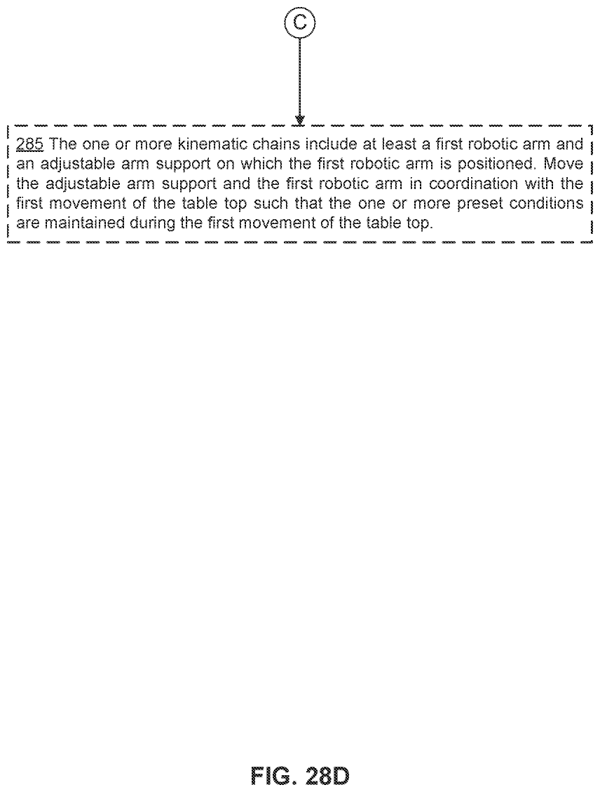

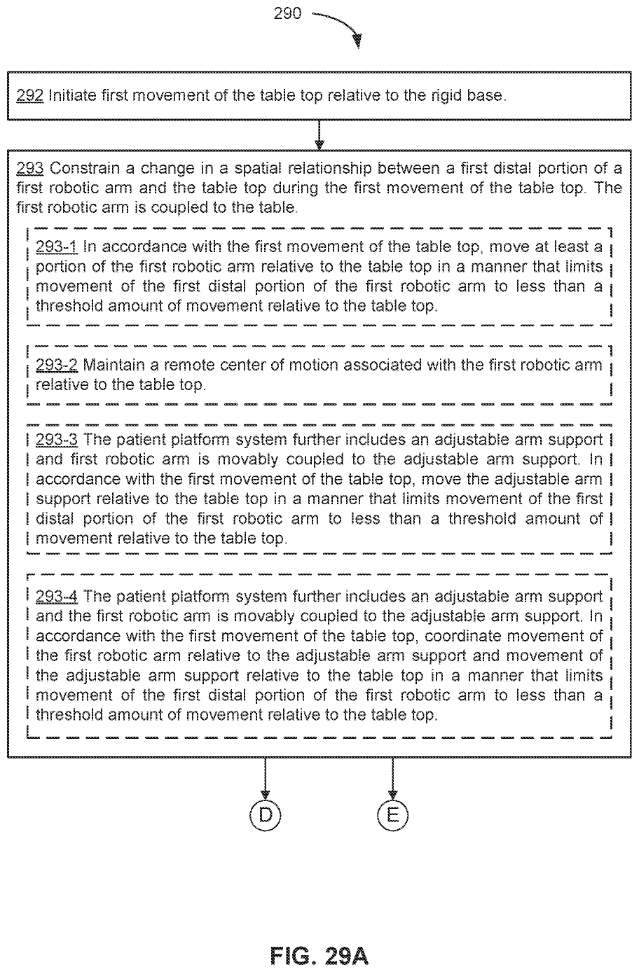

FIGS. 28A-28D show a flow diagram illustrating a method 280 of performing coordinated motion by a patient platform system 200 in accordance with some embodiments. The method 280 is performed at a computer system that is in communication with the patient platform system 200. The computer system includes one or more processors and memory storing one or more programs configured for execution by the one or more processors.

The patient platform system 200 includes a table 202 (e.g., a surgical table, a surgical bed) and one or more kinematic chains 204 that are coupled (e.g., mechanically coupled, movably coupled) to the table 202. The one or more kinematic chains 204 may be directly coupled to the rigid base 224 of the table 202, or coupled to the rigid base 224 via another movable kinematic chain such as an adjustable arm support 210. The table 202 has a rigid base 224 and a table top 225 that is movable (e.g., manually, tele-operatively, and/or automatically movable, capable of tilting, panning, rotating, and translating) relative to the rigid base 224. In some embodiments, the one or more kinematic chains 204 include at least a first robotic arm 205. In some embodiments, the one or more kinematic chains 204 include a first robotic arm 205 and an adjustable arm support 210 on which the first robotic arm 205 is positioned. In some embodiments, the one or more kinematic chains 204 include one or more robotic arms 205, and optionally, adjustable arm support(s) 210 that are respectively coupled to the robotic arms 205. Any of the one or more robotic arm 205 may be in any of: a docked state (e.g., deployed state, in-use state) with or without an attached medical tool 234, and an undocked state (e.g., not-in-use state, retracted state) with or without an attached medical tool 234. In some embodiments, a first robotic arm of the one or more robotic arms 205 is moveably coupled to an adjustable arm support 210 (e.g. a moveable arm support, an arm support that is configured to move relative to the rigid base 224 with one or more degrees of freedom) such that the first robotic arm is movably coupled to the adjustable arm support 210 by a base joint 235-3 that is configured to move relative to the adjustable arm support 210 with one or more degrees of freedom. In some embodiments, the first robotic arm is one of multiple robotic arms that are moveably coupled to the table 202 (e.g., via an adjustable arm support 210). The kinematic chains 204 can be moved and configured manually (e.g., through pure manual manipulation, power-assisted manual manipulation), teleoperatively, and/or automatically based on preprogrammed rules and real-time conditions and sensor information.

The method 280 includes initiating (282) first movement of the table top 225 relative to the rigid base 224 in accordance with a user request, and moving (283) (e.g., automatically moving) the one or more kinematic chains relative to the rigid base 224 in coordination with (e.g., with coordinated timing, movement distance(s), movement direction(s), movement type(s), movement sequence(s)) the first movement of the table top 225 such that one or more preset conditions are maintained during the first movement of the table top 225. For example, the one or more preset conditions may include that movement of the table top 225 and one or more kinematic chains 204 does not result in any of: a collision between kinematic chains 204, a collision between robotic arms 205 of the one or more kinematic chains 204, a load on an ADM 232 of a robotic arm 205 and/or cannula port coupled thereto exceeding a threshold level, movement of the remote center of motion of docked arms with attached tools beyond a threshold amount and a force being exerted on a kinematic chain 204 that is above a threshold force.

In some embodiments, the first movement of the table top 225 includes any movement, such as a preprogrammed fixed movement, a dynamically optimized movement into a preset configuration relative to the rigid base 224, or a movement through direct manual manipulation. For example, the preset configuration may be a Trendelenburg position or another configuration selected or specified by user inputs received via a control user interface or a user input device (such as input device 260).

In some embodiments, automatically moving the one or more kinematic chains in coordination with the first movement of the table top 225 includes determining one or more of a current position, a current movement direction, a current movement type (e.g., rotation, tilt, pan, translation), and/or a current configuration (e.g., overall posture, respective positions and orientations of various portions thereof) of the table top 225 during the first movement of the table top 225, and selecting an updated position and/or configuration (e.g., overall posture, respective positions and orientations of various portions thereof) for at least one of the one or more kinematic chains to maintain the one or more preset conditions.

In some embodiments, automatically moving the one or more kinematic chains in coordination with the first movement of the table top 225 includes determining one or more of a requested position and configuration of the table top 225, generating a first motion sequence to be executed by the table top 225 and second motion sequences to be executed by the one or more kinematic chains. The first motion sequence and the second motion sequences are coordinated in one or more of position, timing, movement direction, movement type, instantaneous configuration, to maintain the one or more preset conditions. The one or more processors then cause the first movement sequence and the second movement sequences to be executed by the table top 225 and the one or more kinematic chains, respectively.

In some embodiments, a respective kinematic chain of the one or more kinematic chains includes one or more sensors for determining a position and/or configuration of the respective kinematic chain. The respective one or more sensors of the respective kinematic chain are configured to provide information regarding any of a position, an orientation, and a load on the respective kinematic chain so that the one or more processors control coordinated movement of the one or more kinematic chains based on the information received from the respective one or more sensors.

In some embodiments, the method 280 includes updating a configuration of the first robotic arm 205 in accordance with the first movement of the table top 225 and in accordance with the one or more preset conditions to be maintained during the first movement of the table top 225.

In some embodiments, the positions and/or configurations of the one or more kinematic chains are determined from commands, instructions, and/or control signals previously provided to respective kinematic chains and/or actuators of the respective kinematic chains.

In some embodiments, the one or more preset conditions that are maintained during the first movement of the table top 225 include a preset condition that limits (283-1) movement of a first distal portion of a kinematic chain (e.g., an ADM 232 of a robotic arm 205 or a medical tool 234 attached to a robotic arm 205) to less than a threshold amount of movement (e.g., threshold amount of change in position and/or orientation) relative to the table top 225.

In some embodiments, the method 280 includes, in accordance with the preset condition, maintaining a remote center of motion 238 of a first robotic arm 205 within a threshold distance of its initial position relative to the table top 225 (e.g., an original position before the first movement of the table top 225 is initiated) while allowing some movement within the threshold distance if the first robotic arm 205 is in a docked state without an attached surgical tool 234, or is in a docked state with a retracted surgical tool 234 and the load on the distal end of the first robotic arm 205 (e.g., on a cannula port attached thereto exceeds a first threshold load (optionally, not exceeding a second threshold greater than the first threshold load). In some embodiments, the preset condition applies to robotic arms 205 that are in a docked state relative to the table top 225 and that do not currently have an attached surgical tool 234. In some embodiments, the preset condition applies to robotic arms 205 that are in a docked state relative to the table top 225 and that have an attached surgical tool 234 that is retracted or not in contact with a patient. In some embodiments, the preset condition applies to robotic arms 205 that are docked relative to the table top 225 and include a surgical tool 234 that is in contact with the patient, for example, extended into the patient or in contact with tissue of the patient. In some embodiments, the patient platform system 200 has multiple robotic arms, and the preset condition is applied to a first subset of the multiple robotic arms (e.g., the robotic arms that are in a docked state and that do not have attached surgical tools at their distal ends, or have retracted tools at their distal ends) and is not applied to a second subset of the multiple robotic arms (e.g., the robotic arms that are in an undocked state relative to the table top 225). In some embodiments, the robotic arms 205 that are undocked relative to the table top 225 are permitted to move without being constrained by their original positions and orientations relative to the table top 225. In some embodiments, the robotic arms 205 that are undocked relative to the table top 225 are configured to be moved manually under an impedance mode or admittance mode (e.g., by a high impact force) during the first movement of the table top 225.

In some embodiments, the one or more preset conditions that are maintained during the first movement of the table top 225 include a preset condition that prohibits (283-2) one or more of: the one or more kinematic chains and the table top 225 from reaching within a threshold distance of one another during the first movement of the table top 225 and kinematic chains of the one or more kinematic chains from reaching within a threshold distance (e.g., a fixed distance, a dynamically variable threshold distance based on a potential well) of one another during the first movement of the table top 225 (e.g., to avoid collision or impact between the moving table top 225 and the one or more robotic arms 205, including robotic arms 205 that are stationary or that are in coordinated motion with the table top 225). In some embodiments, to maintain the preset condition, the method 280 include moving one or more of the robotic arms 205 out of the way to avoid a robotic arm 205 from getting too close to another moving robotic arm or the moving table top 225. In some embodiments, to maintain the preset condition, the instructions cause the one or more processors to change the configurations of one or more of the robotic arms 205 (e.g., during translation along an adjustable arm support 210) to avoid a robotic arm 205 from getting too close to another robotic arm 205 (e.g., moving or stationary) or to the moving table top 225.

In some embodiments, the one or more kinematic chains include a first kinematic chain that includes a first joint 235 (e.g., a joint 235 of a robotic arm 205), and the one or more preset conditions that are maintained during the first movement of the table top 225 include a preset condition that prevents (283-3) the joint 235 from reaching beyond a joint limit.

In some embodiments, the one or more kinematic chains include at least a first robotic arm 205 (e.g., robotic arm 205-1) that has an attached medical tool 234 at its ADM 232 during the first movement of the table top 225, and the one or more preset conditions that are maintained during the first movement of the table top 225 include a preset condition that maintains (283-4) a fixed spatial relationship between the attached medical tool 234 and the table top 225 during the first movement of the table top 225. For example, the one or more preset conditions may require that the relative position and/or relative orientation of a tool tip 236 of the medical tool 234 is maintained during movement of the table top 225.

In some embodiments, the one or more preset conditions include maintaining (283-5) an aligned spatial relationship to a tele-operation input device of the attached medical tool 234 relative to the table top 225.

In some embodiments, the one or more kinematic chains include one or more robotic arms 205 that are in an undocked state relative to the table top 225, and the one or more preset conditions permit (283-6) spatial relationships (e.g., relative positions and orientations) between the table top 225 and respective distal portions of the one or more robotic arms 205 (e.g., the distal portions of the robotic arms 205 that are not coupled to the adjustable arm support, such as ADM(s) 232 of the one or more robotic arms 205 or medical tool(s) 234 attached to any of the one or more robotic arms 205) to change by more than a threshold amount during the first movement of the table top 225. For example, spatial relationships between the distal portion(s) of the one or more undocked robotic arms 205 and the table top 225 may change freely or may change by more than the threshold amount of movement imposed upon docked arms with no attached tools or with retracted tools.

In some embodiments, the one or more kinematic chains include one or more robotic arms 205 that are in an undocked state relative to the table top 225 (and, optionally, one or more robotic arms 205 that are in a docked state relative to the table top 225, and/or one or more adjustable arm supports 210 to which the robotic arms 205 are movably coupled), and the one or more undocked robotic 205 arms remain (284) in non-interfering configurations (e.g., retracted, folded up, fully extended in direction out of the way of other robotic arms 205) during the first movement of the table top 225. For example, in some embodiments, the one or more undocked robotic arms are maintained (e.g., placed and remain) in positions where the one or more undocked robotic arms do not interfere with movement of kinematic chains engaged in coordinated motion with the first movement of the table top 225.

In some embodiments, the one or more kinematic chains include at least a first robotic arm 205 or an adjustable arm support 210 on which the first robotic arm 205 is positioned. The method 280 includes moving the adjustable arm support 210 (e.g., translating and/or rotating of the adjustable arm support 210 in one or more directions relative to the rigid base 224) and the first robotic arm 205 (e.g., translating and/or rotating the first robotic arm 205 in one or more directions relative to the base or the adjustable arm support 210, and/or changing the configuration of the first robotic arm) in coordination with the first movement of the table top 225 such that the one or more preset conditions are maintained during the first movement of the table top 225.

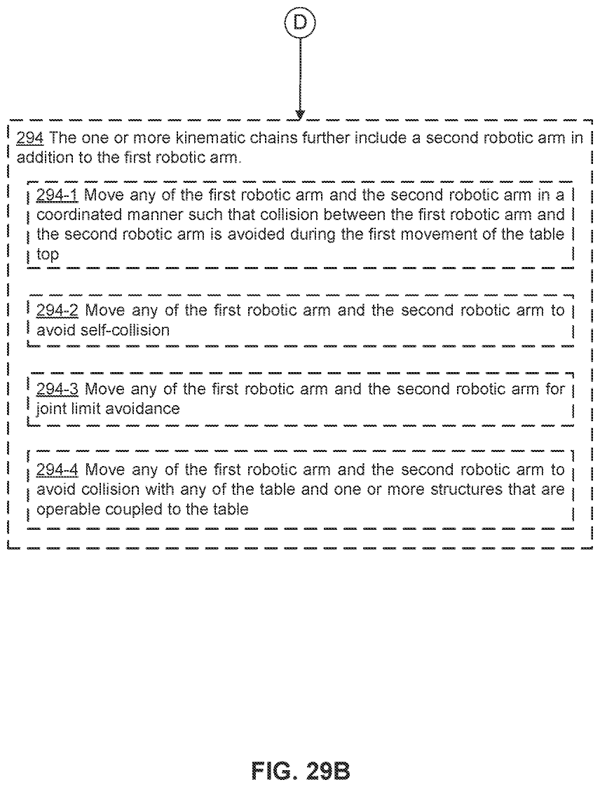

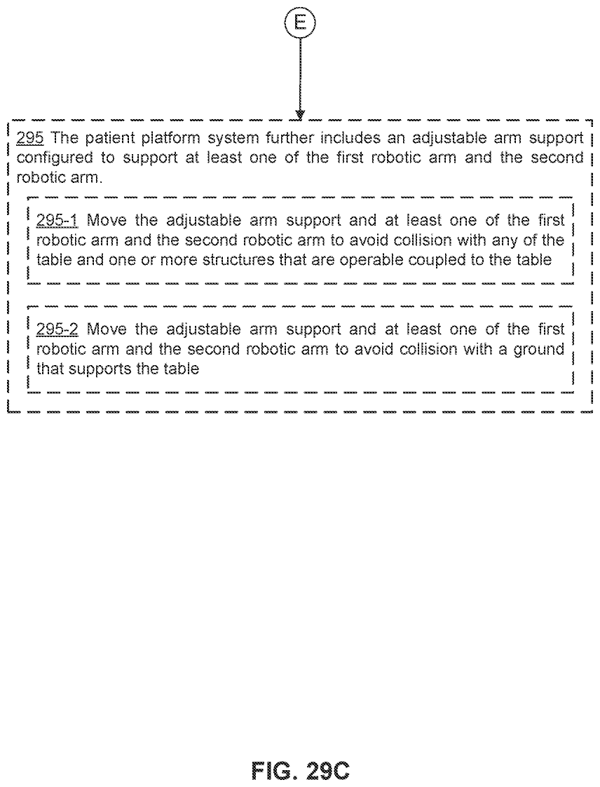

FIGS. 29A-29C show a flow diagram illustrating a method 290 of operating a patient platform system 200 in accordance with some embodiments.

The patient platform system 200 includes a table 202 (e.g., a surgical table, a surgical bed) with a rigid base 224 and a table top 225 that is movable (e.g., manually, tele-operatively, and/or automatically movable, capable of tilting, panning, rotating, and translating) relative to the rigid base 224. The patient platform system 200 also includes a first robotic arm 205 that is coupled (e.g., mechanically coupled, moveably coupled) to the table 202 (e.g., coupled directly to the rigid base 224 of the table 202, to the rigid base 224 via an adjustable arm support 210, to the table top 225). The first robotic arm 205 may be coupled to an adjustable arm support 210. The robotic arm 205 can be moved and configured manually (e.g., through pure manual manipulation, power-assisted manual manipulation), teleoperatively, and/or automatically based on preprogrammed rules and real-time conditions and sensor information.

The method 290 includes initiating (292) first movement of the table top 225 relative to the rigid base 224. The first movement of the table top 225 may include, for example, a preprogrammed fixed movement or dynamically optimized movement of the table top 225 into a preset configuration relative to the rigid base 224. For example, the table top 225 may be moved into a Trendelenburg position or another configuration that is selected or specified by user inputs received via a control user interface. The first movement may also include movement of the table top 225 in response to direct manual manipulation by an operator of the patient platform system 200. In some embodiments, the first movement of the table top 225 is initiated in accordance with a user's request. The user's request may be received via user input(s) at a control device or control user interface (e.g., input device 260), or may be received as manual manipulation of the table top 225 (e.g., while the table top 225 is in a low-impedance mode or an admittance mode). The first movement may be initiated in response to user input corresponding to dynamic user control of the table top 225 via input device(s) or in response a preprogrammed system command.

The method 290 also includes constraining (293) (e.g., through coordinated motion of the first robotic arm 205 and/or adjustable arm support 210 coupled to the first robotic arm 205 that observes one or more preset conditions) a change in a spatial relationship between a first distal portion (e.g., a distal end of the first robotic arm 205 that is configured for attaching a surgical tool, or having an attached surgical tool, such as an ADM 232 of the first robotic arm 205) of the first robotic arm 205 and the table top 225 during the first movement of the table top 225. For example, constraining a change in a spatial relationship between a first distal portion of the first robotic arm 205 and the table top 225 during the first movement of the table top 225 may include limiting an amount of change of a relative position and/or relative orientation of a distal portion of the first robotic arm 205 with respect to the table top 225 to within a preset threshold or a dynamically determined threshold. In another example, constraining a change in a spatial relationship between a first distal portion of the first robotic arm 205 and the table top 225 during the first movement of the table top 225 may include not allowing a relative position and/or relative orientation change of a distal portion of the first robotic arm 205 with respect to the table top 225 to change.

In some embodiments, the method 290 also includes, in accordance with the first movement of the table top 225, moving (293-1) at least a portion of the first robotic arm 205 (e.g., one or more links and joints 235) relative to the table top 225 in a manner (e.g., a manner that observes this and other preset conditions for coordinated motion of one or more kinematic chains of the patient platform system 200) that limits movement of the first distal portion (e.g., the ADM 232, the attached tool 234) of the first robotic arm 205 to less than a threshold amount of movement relative to the table top 225.

In some embodiments, the method 290 includes maintaining (293-2) a remote center of motion 238 associated with the first robotic arm 205 relative to the table top 225.

In some embodiments, the patient platform system 200 further includes an adjustable arm support 210. In some embodiments, the adjustable arm support 210 is movably coupled to the rigid base 224 and movable relative to the table top 225. The method 290 further includes, in accordance with the first movement of the table top 225, moving (293-3) the adjustable arm support 210 relative to the table top 225 in a manner (e.g., a manner that observes this and other preset conditions for coordinated motion of one or more kinematic chains of the patient platform system 200) that limits movement of the first distal portion of the first robotic arm 205 to less than a threshold amount of movement relative to the table top 225. For example, the first robotic arm 205 may be limited to moving the ADM 232 and/or the medical tool 234 attached to the first robotic arm by no more than a predetermined amount (e.g., predetermined distance), such as by no more than 20 mm. In order to maintain the relative position between a distal portion of the robotic arm 205 and the table top 225, the adjustable arm support 210 may be raised to keep the first robotic arm 205 stationary relative to the table top 225 as the table top 225 is moved. In another example, the adjustable arm support 210 may be tilted to keep the first robotic arm 205 stationary relative to the table top 225 as the table top 225 is tilted.

In some embodiments, the patient platform system 200 further includes an adjustable arm support 210. In some embodiments, the adjustable arm support 210 is movably coupled to the rigid base 224 and movable relative to the table top 225. The method 290 further includes, in accordance with the first movement of the table top 225, coordinating (293-4) movement of the first robotic arm 205 relative to the adjustable arm support 210 and movement of the adjustable arm support 210 relative to the table top 225 in a manner (e.g., a manner that observes this and other preset conditions for coordinated motion of one or more kinematic chains of the patient platform system 200) that limits movement of the first distal portion of the first robotic arm 205 to less than a threshold amount of movement relative to the table top 225. For example, the adjustable arm support 210 can move relative to the rigid base 224, and the first robotic arm 205 can move along the adjustable arm support 210 and/or change configuration to keep the first distal portion of the first robotic arm 205 stationary or within a preset range relative to the table top 225.

In some embodiments, the one or more kinematic chains 204 further include (294) a second robotic arm in addition to the first robotic arm (e.g., separately controllable robotic arms that are coupled to the same adjustable arm support 210, such as robotic arms 205-3 and 205-4 shown in FIGS. 25A-25C, or different adjustable arm supports, such as robotic arms 205-1 and 205-2 shown in FIGS. 24A-24C).

In some embodiments, the method 290 includes (a) moving (294-1) any of the first robotic arm and the second robotic arm (e.g., relative to the rigid base 224, relative to the table top 225, relative to the adjustable arm support(s) 210, relative to one another) in a coordinated manner such that collision between the first robotic arm and the second robotic arm is avoided during the first movement of the table top 225. For example, the first robotic arm and/or the second robotic arm may be moved in a coordinated manner such that impact or contact between a portion of the first robotic arm and a portion of the second robotic arm is avoided, a distance between respective portions of the first robotic arm and respective portions of the second robotic arm remain greater than a threshold distance, and/or a force between respective portions of the first robotic arm and respective portions of the second robotic arm remain below a threshold force. For example, referring to robotic arms 205-1 and 205-2 shown in FIGS. 24A-24C, performing coordinated motion includes moving any of the robotic arms 205-1 and 205-2 so that the two robotic arms 205-1 and 205-2 do not collide or come into contact with one another. In another example, referring to FIGS. 25A-25C, performing coordinated motion includes moving any of the robotic arms 205-3, 205-4, and 205-5 so that the none of the robotic arms 205-3, 205-4, and 205-5 collide or come into contact with one another.

In some embodiments, the method 290 includes moving (294-2) any of the first robotic arm and the second robotic arm (e.g., robotic arms 205) to avoid self-collision (e.g., avoid a portion of the robotic arm 205 from making contact with another portion of same the robotic arm 205).

In some embodiments, the method 290 includes moving (294-3) any of the first robotic arm and the second robotic arm (e.g., robotic arms 205) for joint limit avoidance (e.g., so that a joint 235 of a robotic arm 205 does not exceed a joint limit threshold).

In some embodiments, the method 290 includes moving (294-4) any of the first robotic arm and the second robotic arm (e.g., robotic arms 205) to avoid collision with any of the table 225 and one or more structures that are operable coupled to the table. For example, referring to FIGS. 24A-24C, which show two robotic arms 205-1 and 205-2, performing coordinated motion includes moving any of the robotic arms 205-1 and 205-2 so that the two robotic arms 205-1 and 205-2 do not collide or come into contact with the table top 225. In another example, referring to FIGS. 25A-25C, performing coordinated motion includes moving any of the robotic arms 205-3, 205-4, and 205-5 so that the none of the robotic arms 205-3, 205-4, and 205-5 collide or come into contact with one another or the table top 225 or other structures coupled to the table top 225 such as an adjustable arm support 210, rigid base 224, and/or a bed column 203.

In some embodiments, the method 290 includes any combination of operations 294-1 through 294-4.

In some embodiments, the patient platform system 200 further includes (295) an adjustable arm support 210 configured to support at least one of the first robotic arm or the second robotic arm (e.g., robotic arms 205), and the method 290 includes any of: (e) moving (295-1) the adjustable arm support 210 and at least one of the first robotic arm or the second robotic arm to avoid collision with any of the table 202 and one or more structures that are operable coupled to the table 202, and (f) moving the adjustable arm support 210 and at least one of the first robotic arm or the second robotic arm to avoid collision with a ground that supports the table 202.

In some embodiments, the first robotic arm includes at least one kinematically redundant joint that is configured to move (e.g., translate, rotate) while the change in spatial relationship between the first distal portion (e.g., the ADM 232, the attached tool 234) of the first robotic arm 205 and the table top 225 is constrained (e.g., while the first distal portion of the first robotic arm 205 is maintained in a stationary position or within a threshold range of its original position/orientation relative to the table top 225). For example, the first robotic arm 205 has a higher number of degrees of freedom than required to perform medical tasks (e.g., the first robotic arm 205, with or without an associated adjustable arm support 210, may have seven, eight, or nine degrees of freedom or higher) and thus, is configurable to perform a null-space motion where portions of the first robotic arm 205 may be moved without moving (or with negligible movement to) a distal portion of the robotic arm 205.

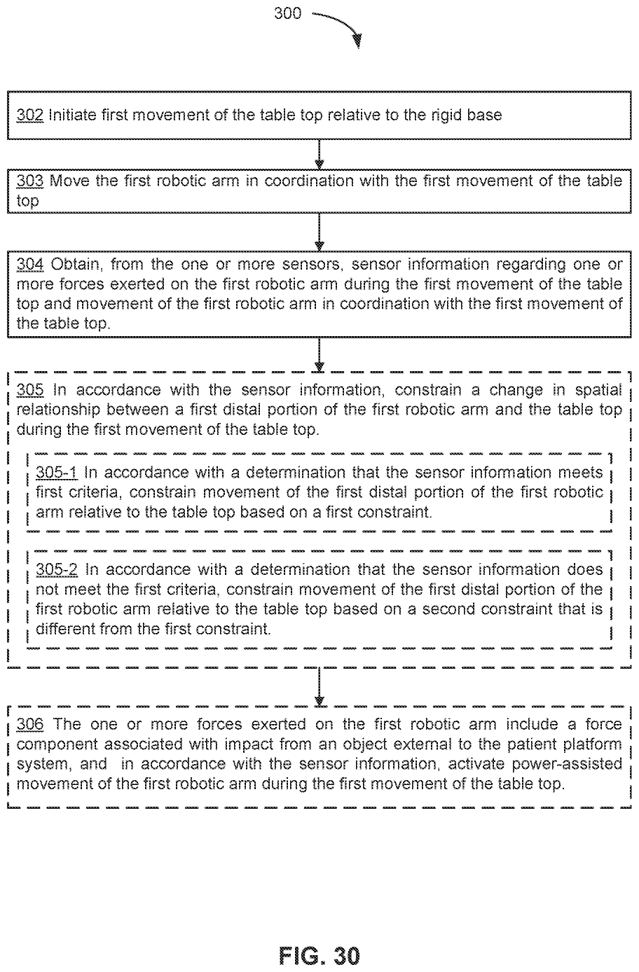

FIG. 30 shows a flow diagram illustrating a method 300 of operating a patient platform system 200 in accordance with some embodiments.

The patient platform system 200 includes a first robotic arm 205, a table 202, and one or more sensors (e.g., force sensors, torque sensors, contact sensors, load cells). The table 202 includes a rigid base 224 and a table top 225 that is movable relative to the rigid base 224. The one or more sensors are positioned to detect one or more forces (e.g., force or physical manifestation of exerted force, sheer, friction, torque, deformation, contact, pressure, load) exerted on the first robotic arm 205 (e.g., on the surface, on a joint 235, and/or on a link of the first robotic arm). The one or more sensors may be located on or underneath the surface of one or more links of the first robotic arm 205, on or inside one or more joints 235 of the first robotic arm 205, and/or on or inside a distal end of the first robotic arm 205. In some embodiments, the one or more sensors include sensors that are located on one or more components (e.g., on an adjustable arm support 210, on the rigid base 224) that are coupled to the first robotic arm 205.

The method 300 includes initiating (302) first movement of the table top 225 relative to the rigid base 224, and moving (303) the first robotic arm 205 in coordination with the first movement of the table top 225. The method 300 also includes obtaining (304), from the one or more sensors, sensor information regarding one or more forces exerted on the first robotic arm 205 during the first movement of the table top 225 and movement of the first robotic arm 205 in coordination with the first movement of the table top 225.

In some embodiments, the one or more forces exerted on the first robotic arm 205 include a force component associated with gravity of a patient positioned on the table top 225. For example, a force exerted on a distal end of the first robotic arm 205 is due to a portion of the patient being in contact with the distal end of the first robotic arm 205 (e.g., due to the weight of the patient or portion thereof).

In some embodiments, the method 300 also includes in accordance with the sensor information, constraining (305) (e.g., through coordinated motion of the first robotic arm 205 and/or an adjustable arm support 210 coupled to the first robotic arm 205) a change in spatial relationship between a first distal portion of the first robotic arm 205 (e.g., the ADM 232 of the first robotic arm 205 that is configured for attaching a surgical tool 234, or having an attached surgical tool 234) and the table top 225 during the first movement of the table top 225. For example, constraining a change in spatial relationship between a first distal portion of the first robotic arm 205 and the table top 225 during the first movement of the table top 225 may include limiting the change in relative position and/or orientation to no change at all, to a preset threshold amount of change, or to a dynamically determined threshold amount of change.

In some embodiments, the method 300 includes, in accordance with a determination that the sensor information meets first criteria (e.g., force exceeds a preset threshold force, force in a preset direction exceeds a first preset threshold force, and/or force on the first distal end of the first robotic arm exceeds a preset threshold force), constraining (305-1) movement of the first distal portion (e.g., the ADM 232, the attached tool 234) of the first robotic arm 205 relative to the table top 225 based on a first constraint (e.g., a relaxed constraint, not maintaining a fixed spatial relationship, permitting change within a first threshold range of change).

In some embodiments, the method 300 includes, in accordance with a determination that the sensor information does not meet first criteria (e.g., force does not exceed the preset threshold force, force in the preset direction does not exceed the first preset threshold force, and/or force on the first distal end of the first robotic arm does not exceed the preset threshold force), constraining (305-2) (e.g., in a strict manner, a strict constraint, maintaining a fixed spatial relationship, permitting change within a second threshold range that is smaller than the first threshold range of change) movement of the first distal portion (e.g., the ADM 232, the attached tool 234) of the first robotic arm 205 relative to the table top 225 based on a second constraint (e.g., a stringent constraint) that is different from the first constraint (e.g., more restrictive than the first constraint).

In some embodiments, the first robotic arm 205 includes an attached surgical tool 234 that is retracted from the first distal portion of the first robotic arm 205 away from the table top 225.

In some embodiments, the first constraint (e.g., the relaxed constraint) is only applied if the surgical tool is retracted from a docked robotic arm. If the surgical tool is not retracted, the first distal portion of the first robotic arm 205 is constrained by the second, more stringent, constraint even if the force on the first distal end exceeds the preset force threshold set by the first criteria.

In some embodiments, the one or more forces exerted on the first robotic arm 205 include (306) a force component associated with impact (e.g., intentional pushing, pulling, inadvertent bumping) from an object external to the patient platform system 200 (such as a patient supported by the patient platform system 200). For example, a force exerted may be exerted on the first robotic arm 205 by the patient's arm or by medical personnel on the surface or joint 235 of the first robotic arm 205. The method 300 includes activating power-assisted movement of the first robotic arm 205 during the first movement of the table top 225 in accordance with the sensor information.

In some embodiments, the method 300 further includes activating power-assisted movement of the first robotic arm (e.g., manual movement under impedance mode, admittance mode) during the first movement of the table top in accordance with the sensor information. For example, in accordance with a determination that the force component associated with impact from an object external to the patient platform system 200 exceeds a preset force threshold, and that the first robotic arm 205 is undocked relative to the table top 225. In another example, in some embodiments, at least a portion of the first robotic arm 205 moves out of the way to resolve impact or pressure on the first robotic arm 205 from an external object (and optionally, from other robotic arms 205). In some embodiments, the movement is a null-space movement that maintains a position of the remote center of motion 238 of top first robotic arm 205 with respect to the table top 225 within a threshold range (e.g., when the first robotic arm 205 is docked relative to the table top). In some embodiments, the movement is not constrained by the position and orientation of the remote center of motion 238 of the first robotic arm 205 if the first robotic arm 205 is not docked.

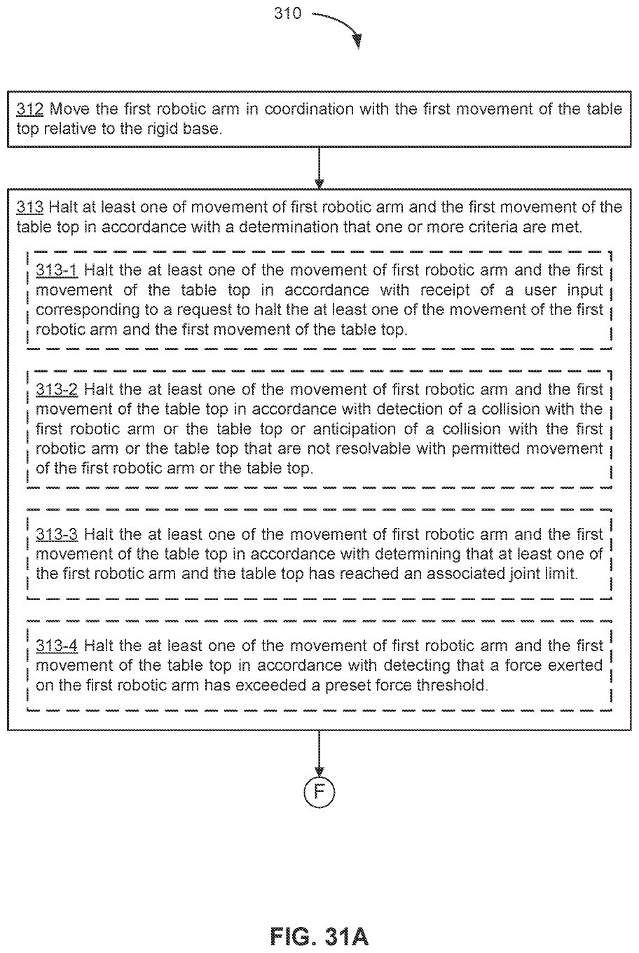

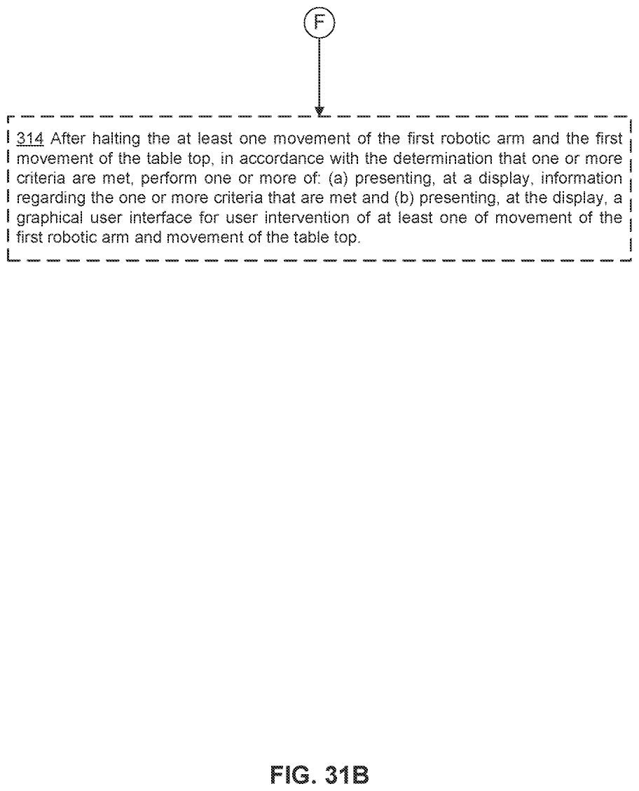

FIGS. 31A-31B show a flow diagram illustrating a method 310 of operating a patient platform system 200 in accordance with some embodiments. The patient platform system 200 includes a first robotic arm 205 and a table 202. The table 202 includes a rigid base 224 and a table top 225.

The method 310 includes moving (312) the first robotic arm 205 in coordination with the first movement of the table top 225 relative to the rigid base 224. In some embodiments, movement of the table top 225 is performed in accordance with a user's request (e.g., in response to user input received via a control device or control user interface and/or in response to manual manipulation of the table top 225 while the patient platform system 200 is in an impedance mode or an admittance mode. In some embodiments, movement of the table top 225 is performed in response to a preprogrammed system command.

The method 310 also includes halting (313) at least one of movement of first robotic arm 205 or the first movement of the table top 225 in accordance with a determination that one or more criteria are met.

In some embodiments, the method 310 includes halting (313-1) the at least one of the movement of first robotic arm 205 or the first movement of the table top 225 in accordance with receipt of (e.g., receiving, detecting) a user input corresponding to a request to halt the at least one of the movement of the first robotic arm 205 or the first movement of the table top 225. For example, movement of the table top 225 and/or the first robotic arm 205 may be halted in response to receiving an active user input (e.g., activating a control, a voice command) or cessation of a continuously maintained user input (e.g., user releases a button, liftoff from a touch-sensitive surface).

In some embodiments, the method 310 includes halting (313-2) the at least one of the movement of first robotic arm 205 or the first movement of the table top 225 in accordance with detection of a collision with the first robotic arm 205 or the table top 225 (e.g., a collision between robotic arms, a collision with the patient or medical personnel, a collision with the table top 225) or anticipation of a collision with the first robotic arm 205 or the table top 225 that are not resolvable with permitted movement of the first robotic arm 205 or the table top 225 (e.g., null space movement, and/or coordinated movement that observe the one or more preset conditions).

In some embodiments, the method 310 includes halting (313-3) the at least one of the movement of first robotic arm 205 or the first movement of the table top 225 in accordance with determining that at least one of the first robotic arm 205 or the table top 225 has reached an associated joint limit (e.g., a positional limit, an angular limit).

In some embodiments, the method 310 includes halting (313-4) the at least one of the movement of first robotic arm 205 or the first movement of the table top 225 in accordance with detecting that a force exerted on the first robotic arm 205 has exceeded a preset force threshold (e.g., in the case where the first robotic arm 205 is docked and has an attached tool 234 that is not retracted away from the table top 225).

In some embodiments, the method 310 further includes, after halting the at least one of movement of the first robotic arm 205 or the first movement of the table top 225, in accordance with the determination that one or more criteria are met, performing (314) one or more of: (a) presenting, at the display (such as display 264 on input device 260), information regarding the one or more criteria that are met or (b) presenting, at the display, a graphical user interface for user intervention of at least one of movement of the first robotic arm 205 or movement of the table top 225.

In some embodiments, the method 310 includes, after halting the at least one of movement of the first robotic arm 205 or the first movement of the table top 225, providing an indication (e.g., via a display 264 or an indicator) to a user corresponding to a reason for halting the movement of the first robotic arm 205 and/or the first movement of the table top 225. In some embodiments, the method 310 includes, after halting the at least one of movement of the first robotic arm 205 or the first movement of the table top 225, displaying a user interface (e.g., via a display such as display 264) that facilitates user intervention to resolve any safety issues or violations of preset conditions that have been detected. For example, the patient platform system 200 may, after halting movement of the table top 225 and/or movement of the robotic arm 205, visually indicate (e.g., highlight or emphasize) two robotic arms 205 of the patient platform system 200 that are anticipated to collide with one another if the coordinated motion were to continue to be performed. The patient platform system 200 may also provide a suggested movement of the robotic arms 205 and/or a user interface to control movement of the robotic arms 205 so that the user may move at least one of the robotic arms 205 thereby resolving the anticipated collision.

In accordance with some embodiments, a patient platform system 200 includes a table 202 with a rigid base 224 and a table top 225 that is movable relative to the rigid base 224, one or more kinematic chains 204 that are coupled to the table 202, one or more processors, and memory storing instructions. The instructions, when executed by the one or more processors, cause the one or more processors to initiate first movement of the table top 225 relative to the rigid base 224 in accordance with a user request, and move the one or more kinematic chains 204 relative to the rigid base 224 in coordination with the first movement of the table top 225 such that one or more preset conditions are maintained during the first movement of the table top 225.

In some embodiments, the one or more kinematic chains 204 include at least a first robotic arm 205.

In some embodiments, the one or more preset conditions that are maintained during the first movement of the table top 225 include a preset condition that limits movement of a first distal portion (e.g., an ADM 232, a remote center of motion 238, a tool tip position of a medical tool 234) of a first kinematic chain 204 to less than a threshold amount of movement relative to the table top 225.

In some embodiments, the preset condition that limits the movement of the first distal portion of the first kinematic chain 204 to less than the threshold amount of movement relative to the table top 225 includes maintaining a remote center of motion 238 associated with the first kinematic chain 204 relative to the table top 225.

In some embodiments, the one or more preset conditions that are maintained during the first movement of the table top 225 include a preset condition that prohibits one or more of: the one or more kinematic chains 204 and the table top 225 from reaching within a threshold distance of one another during the first movement of the table top 225, or the kinematic chains 204 of the one or more kinematic chains from reaching within a threshold distance of one another during the first movement of the table top 225.

In some embodiments, the one or more preset conditions that are maintained during the first movement of the table top 225 include a preset condition that prevents the one or more kinematic chains 204 from reaching within a threshold distance of one or more objects adjacent to the patient platform system 200.

In some embodiments, the one or more kinematic chains 204 include a first kinematic chain that includes a first joint (e.g., a robotic arm 205 that includes a joint 235, an adjustable arm support 210). The one or more preset conditions that are maintained during the first movement of the table top 225 include a preset condition that prevents the first joint from reaching beyond a joint limit (e.g., a joint limit of a joint 235 of a robotic arm 205, a joint limit of an adjustable arm support 210).

In some embodiments, the one or more kinematic chains 204 include at least a first robotic arm 205 that has an attached medical tool 234 at its distal end (e.g., ADM 232) during the first movement of the table top 225.

In some embodiments, the one or more preset conditions that are maintained during the first movement of the table top 225 include a preset condition that maintains a fixed spatial relationship between the attached medical tool 234 and the table top 225 during the first movement of the table top 225.

In some embodiments, the one or more preset conditions include maintaining an aligned spatial relationship to a tele-operation input device of the attached medical tool 234 relative to the table top 225.

In some embodiments, the one or more kinematic chains 204 include a first robotic arm 205 and an adjustable arm support 210 on which the first robotic arm 205 is positioned.

In some embodiments, the instructions, when executed by the one or more processors, cause the one or more processors to move the adjustable arm support 210 and the first robotic arm 205 in coordination with the first movement of the table top 225 such that the one or more preset conditions are maintained during the first movement of the table top 225.

In some embodiments, the one or more kinematic chains 204 include one or more robotic arms 205 that are in an undocked state relative to the table top 225. The one or more preset conditions permit spatial relationships between the table top 225 and respective distal portions of the one or more robotic arms 205 to change by more than a threshold amount during the first movement of the table top 225.

In some embodiments, the one or more kinematic chains 204 include one or more robotic arms 205 that are in an undocked state relative to the table top 225. The one or more undocked robotic arms remain in non-interfering configurations during the first movement of the table top 225.

In accordance with some embodiments, a non-transitory computer readable storage medium stores one or more programs configured for execution by a computer system that has one or more processors, memory, and a display. The one or more programs include instructions for receiving a user request to move a table top 225 of a patient platform system 200. The patient platform system 200 includes a table 202 with the table top 225 and a rigid base 224, and the table top 225 is movable relative to a rigid base 224. The one or more programs also include instructions for initiating first movement of the table top 225 relative to a rigid base 224 in accordance with the user request, and moving one or more kinematic chains 204 relative to the rigid base 224 in coordination with the first movement of the table top 225 such that one or more preset conditions are maintained during the first movement of the table top 225. The one or more kinematic chains 204 are coupled to the table 202.

In accordance with some embodiments, a patient platform system 200 includes a table 202 with a rigid base 224 and a table top 225 that is movable relative to the rigid base 224, a first robotic arm 205 that is coupled to the table 202, one or more processors, and memory storing instructions. The instructions, when executed by the one or more processors, cause the one or more processors to initiate first movement of the table top 225 relative to the rigid base 224, and constrain a change in spatial relationship between a first distal portion of the first robotic arm 205 and the table top 225 during the first movement of the table top 225.

In some embodiments, constraining the change in spatial relationship between the first distal portion of the first robotic arm 205 and the table top 225 during the first movement of the table top 225 includes, in accordance with the first movement of the table top 225, moving at least a portion of the first robotic arm 205 relative to the table top 225 in a manner that limits movement of the first distal portion of the first robotic arm 205 to less than a threshold amount of movement relative to the table top 225.

In some embodiments, constraining the change in spatial relationship between the first distal portion of the first robotic arm 205 and the table top 225 during the first movement of the table top 225 includes maintaining a remote center of motion 238 associated with the first robotic arm 205 relative to the table top 225.

In some embodiments, the patient platform system 200 further includes an adjustable arm support 210, and the first robotic arm 205 is movably coupled to the adjustable arm support 210. Constraining the change in spatial relationship between a first distal end of the first robotic arm 205 and the table top 225 during the first movement of the table top 225 includes, in accordance with the first movement of the table top 225, moving the adjustable arm support 210 relative to the table top 225 in a manner that limits movement of the first distal portion of the first robotic arm 205 to less than a threshold amount of movement relative to the table top 225.

In some embodiments, the patient platform system 200 further includes an adjustable arm support 210, and the first robotic arm 205 is movably coupled to the adjustable arm support 210. Constraining the change in spatial relationship between a first distal end of the first robotic arm 205 and the table top 225 during the first movement of the table top 225 includes, in accordance with the first movement of the table top 225, coordinating movement of the first robotic arm 205 relative to the adjustable arm support 210 and movement of the adjustable arm support 210 relative to the table top 225 in a manner that limits movement of the first distal portion of the first robotic arm 205 to less than a threshold amount of movement relative to the table top 225.

In some embodiments, the first robotic arm 205 includes at least one kinematically redundant joint that is configured to move while the change in spatial relationship between the first distal portion of the first robotic arm 205 and the table top 225 is constrained.

In some embodiments, the patient platform system 200 further includes a second robotic arm in addition to the first robotic arm. The stored instructions, when executed by the one or more processors, cause the one or more processors to perform any of (a) moving any of the first robotic arm and the second robotic arm in a coordinated manner such that collision between the first robotic arm and the second robotic arm is avoided during the first movement of the table top 225, (b) moving any of the first robotic arm and the second robotic arm to avoid self-collision, (c) moving any of the first robotic arm and the second robotic arm for joint limit avoidance, and (d) moving any of the first robotic arm and the second robotic arm to avoid collision with any of the table 202 and one or more structures (e.g., bed column 203, set-up joints 215, adjustable arm support 210) that are operable coupled to the table 202.

In some embodiments, the patient platform system 200 further includes an adjustable arm support 210 configured to support at least one of the first robotic arm or the second robotic arm. The stored instructions, when executed by the one or more processors, cause the one or more processors to perform any of (e) moving the adjustable arm support 210 and at least one of the first robotic arm or the second robotic arm to avoid collision with any of the table 202 and one or more structures that are operable coupled to the table 202, and (f) moving the adjustable arm support 210 and at least one of the first robotic arm or the second robotic arm to avoid collision with a ground that supports the table 202.

In accordance with some embodiments, a non-transitory computer readable storage medium stores one or more programs configured for execution by a computer system that has one or more processors, memory, and a display. The one or more programs include instructions for initiating first movement of a table top 225 of a patient platform system 200. The patient platform system 200 includes a table 202 with a rigid base 224 and the table top 225, and the table top 225 is movable relative to the rigid base 224. The one or more programs also include instructions for constraining a change in spatial relationship between a first distal portion of a first robotic arm 205 and the table top 225 during the first movement of the table top 225. The first robotic arm 205 is coupled to the table 202.

In accordance with some embodiments, a patient platform system 200 includes a table 202 with a rigid base 224 and a table top 225 that is movable relative to the rigid base 224, a first robotic arm 205, one or more sensors that are positioned to detect one or more forces exerted on the first robotic arm 205, one or more processor, and memory storing instructions. The instructions, when executed by the one or more processors, cause the one or more processors to initiate first movement of the table top 225 relative to the rigid base 224, move the first robotic arm 205 in coordination with the first movement of the table top 225, and obtain sensor information from the one or more sensors. The sensor information includes information regarding one or more forces exerted on the first robotic arm 205 during the first movement of the table top 225 and movement of the first robotic arm 205 in coordination with the first movement of the table top 225.

In some embodiments, the one or more forces exerted on the first robotic arm 205 include a force component associated with gravity of a patient positioned on the table top 225.

In some embodiments, the stored instructions, when executed by the one or more processors, cause the one or more processors to, in accordance with the sensor information, constrain a change in spatial relationship between a first distal portion of the first robotic arm 206 and the table top 225 during the first movement of the table top 225.

In some embodiments, constraining the change in spatial relationship between the first distal portion of the first robotic arm 205 and the table top 225 during the first movement of the table top 225 includes: in accordance with a determination that the sensor information meets first criteria, constraining movement of the first distal portion of the first robotic arm 205 relative to the table top 225 based on a first constraint, and in accordance with a determination that the sensor information does not meet the first criteria, constraining movement of the first distal portion of the first robotic arm 205 relative to the table top 225 based on a second constraint that is different from the first constraint.

In some embodiments, the first robotic arm 205 includes an attached surgical tool 234 that is retracted from the first distal portion of the first robotic arm 205 away from the table top 225.

In some embodiments, the one or more forces exerted on the first robotic arm 205 include a force component associated with impact from an object external to the patient platform system 200.

In some embodiments, the stored instructions further include instructions, which, when executed by the one or more processors, cause the one or more processors to, in accordance with the sensor information, activate power-assisted movement of the first robotic arm 205 during the first movement of the table top 225.

In accordance with some embodiments, a non-transitory computer readable storage medium stores one or more programs configured for execution by a computer system that has one or more processors, memory, and a display. The one or more programs include instructions for initiating first movement of a table top 225 of a patient platform system 200. The patient platform system includes a first robotic arm 205, a table 202 with a rigid base 224 and a table top 225, and one or more sensors positioned to detect one or more forces exerted on the first robotic arm 205. The table top 225 is movable relative to the rigid base 224. The one or more programs also include instructions for moving the first robotic arm 205 in coordination with the first movement of the table top 225, and obtaining sensor information from one or more sensors that are positioned to detect one or more forces exerted on the first robotic arm 205. The sensor information includes information regarding one or more forces exerted on the first robotic arm 205 during the first movement of the table top 225 and movement of the first robotic arm 205 in coordination with the first movement of the table top 225.

In accordance with some embodiments, a patient platform system 200 includes a table 202 with a rigid base 224 and a table top 225 that is movable relative to the rigid base 224, a first robotic arm 205, one or more processors, and memory storing instructions. The instructions, when executed by the one or more processors, cause the one or more processors to move the first robotic arm 205 in coordination with first movement of the table top 225 relative to the rigid base 224 and halt at least one of movement of first robotic arm 205 or the first movement of the table top 225 in accordance with a determination that one or more criteria are met.

In some embodiments, halting the at least one of the movement of first robotic arm 205 or the first movement of the table top 225 in accordance with the determination that the one or more criteria are met includes halting the at least one of the movement of first robotic arm 205 or the first movement of the table top 225 in accordance with receipt of a user input corresponding to a request to halt the at least one of the movement of the first robotic arm 205 or the first movement of the table top 225.

In some embodiments, halting the at least one of the movement of first robotic arm 205 or the first movement of the table top 225 in accordance with the determination that the one or more criteria are met includes halting the at least one of the movement of first robotic arm 205 or the first movement of the table top 225 in accordance with detection of a collision with the first robotic arm 205 or the table top 225 or anticipation of a collision with the first robotic arm 205 or the table top 225 that are not resolvable with permitted movement of the first robotic arm 205 or the table top 225.

In some embodiments, halting the at least one of the movement of first robotic arm 205 or the first movement of the table top 225 in accordance with a determination that the one or more criteria are met includes halting the at least one of the movement of first robotic arm 205 or the first movement of the table top 225 in accordance with determining that at least one of the first robotic arm 205 or the table top 225 has reached an associated joint limit.

In some embodiments, halting the at least one of the movement of first robotic arm 205 or the first movement of the table top 225 in accordance with a determination that the one or more criteria are met includes halting the at least one of the movement of first robotic arm 205 or the first movement of the table top 225 in accordance with detecting that a force exerted on the first robotic arm 205 has exceeded a preset force threshold.

In some embodiments, the patient platform system 200 further includes a display (e.g., display 264). The stored instructions further include instructions, which, when executed by the one or more processors, cause the one or more processors to, after halting the at least one of movement of the first robotic arm 205 or the first movement of the table top 225 and in accordance with the determination that one or more criteria are met, present information regarding the one or more criteria that are met at the display 264, and/or present a graphical user interface for user intervention of movement of the first robotic arm 205 and/or movement of the table top 225.

In accordance with some embodiments, a non-transitory computer readable storage medium stores one or more programs configured for execution by a computer system that has one or more processors, memory, and a display. The one or more programs include instructions for moving a first robotic arm 205 in coordination with first movement of a table top 225 relative to a rigid base 224, and halting at least one of movement of first robotic arm 205 or the first movement of the table top 225 in accordance with a determination that one or more criteria are met.

### 3. Implementing Systems and Terminology

FIG. 32 is a schematic diagram illustrating electronic components of the patient platform system 200 in accordance with some embodiments.

The patient platform system 200 includes one or more processors 320, which are in communication with a computer readable storage medium 322 (e.g., computer memory devices, such as random-access memory, read-only memory, static random-access memory, and non-volatile memory, and other storage devices, such as a hard drive, an optical disk, a magnetic tape recording, or any combination thereof) storing instructions for performing any methods described herein (e.g., operations described with respect to FIGS. 28A-28D, 29A-29C, 30, and 31A-31B). The one or more processors 320 are also in communication with an input/output controller 324 (via a system bus or any electrical circuit). The input/output controller 324 receives instructions and/or data from an input device (e.g., a user input device 326 that corresponds to input device 260) and relays the received instructions and/or data to the one or more processors 320 (e.g., with or without any translation, conversion, and/or data processing). The input/output controller 324 also receives instructions and/or data from the one or more processors 320 and relays the instructions and/or data to one or more actuators, such as mechanisms 330 responsible for motion of components of the patient platform system 200 (e.g., mechanisms for movement of the table top 225, the robotic arms 205, the adjustable arm supports 210, etc.). In some embodiments, the input/output controller 324 is coupled to one or more actuator controllers 332 and provides instructions and/or data to at least a subset of the one or more actuator controllers 332, which, in turn, provide control signals to selected actuators. In some embodiments, the one or more actuator controllers 332 are integrated with the input/output controller 324 and the input/output controller 324 provides control signals directly to the one or more actuators (without a separate actuator controller). Although FIG. 32 shows one actuator controller 332, in some embodiments, any number of actuator controllers may be used (e.g., one actuator controller for the entire patient platform system 200, or one actuator controller for each robotic arm 205).

Implementations disclosed herein provide systems, methods and apparatus for coordinated movement between a table top and one or more robotic arms of a patient platform system.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The coordinated motion of a table top and one or more robotic arms of a patient platform system described herein may be stored as one or more instructions on a processor-readable or computer-readable medium. The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

The previous description of the disclosed implementations is provided to enable any person skilled in the art to make or use the present invention. Various modifications to these implementations will be readily apparent to those skilled in the art, and the generic principles defined herein may be applied to other implementations without departing from the scope of the invention. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present invention is not intended to be limited to the implementations shown herein but is to be accorded the widest scope consistent with the principles and novel features disclosed herein.

Some embodiments or implementations are described with respect to the following clauses:

Clause 1. A patient platform system comprising: a table with a rigid base and a table top that is movable relative to the rigid base; one or more kinematic chains that are coupled to the table; one or more processors; and memory storing instructions, which, when executed by the one or more processors, cause the one or more processors to: initiate first movement of the table top relative to the rigid base in accordance with a user request; and move the one or more kinematic chains relative to the rigid base in coordination with the first movement of the table top such that one or more preset conditions are maintained during the first movement of the table top.

Clause 2. The patient platform system of Clause 1, wherein the one or more kinematic chains include at least a first robotic arm.

Clause 3. The patient platform system of Clause 1 or 2, wherein the one or more preset conditions that are maintained during the first movement of the table top include a preset condition that limits movement of a first distal portion of a first kinematic chain to less than a threshold amount of movement relative to the table top.

Clause 4. The patient platform system of Clause 3, wherein the preset condition that limits the movement of the first distal portion of the first kinematic chain to less than the threshold amount of movement relative to the table top comprises maintaining a remote center of motion associated with the first kinematic chain relative to the table top.

Clause 5. The patient platform system of any of Clauses 1-4, wherein the one or more preset conditions that are maintained during the first movement of the table top include a preset condition that prohibits one or more of: the one or more kinematic chains and the table top from reaching within a threshold distance of one another during the first movement of the table top; or kinematic chains of the one or more kinematic chains from reaching within a threshold distance of one another during the first movement of the table top.

Clause 6. The patient platform system of any of Clauses 1-5, wherein the one or more preset conditions that are maintained during the first movement of the table top include a preset condition that prevents the one or more kinematic chains from reaching within a threshold distance of one or more objects adjacent to the patient platform system.

Clause 7. The patient platform system of any of Clauses 1-6, wherein the one or more kinematic chains include a first kinematic chain that includes a first joint, wherein the one or more preset conditions that are maintained during the first movement of the table top include a preset condition that prevents the first joint from reaching beyond a joint limit.

Clause 8. The patient platform system of any of Clauses 1-7, wherein the one or more kinematic chains include at least a first robotic arm that has an attached medical tool at its distal end during the first movement of the table top.

Clause 9. The patient platform system of Clause 8, wherein the one or more preset conditions that are maintained during the first movement of the table top include a preset condition that maintains a fixed spatial relationship between the attached medical tool and the table top during the first movement of the table top.

Clause 10. The patient platform system of Clause 8 or 9, wherein the one or more preset conditions maintain an aligned spatial relationship to a tele-operation input device of the attached medical tool relative to the table top.

Clause 11. The patient platform system of any of Clauses 1-10, wherein the one or more kinematic chains include a first robotic arm and an adjustable arm support on which the first robotic arm is positioned.

Clause 12. The patient platform system of Clause 11, wherein the instructions, when executed by the one or more processors, cause the one or more processors to move the adjustable arm support and the first robotic arm in coordination with the first movement of the table top such that the one or more preset conditions are maintained during the first movement of the table top.

Clause 13. The patient platform system of any of Clauses 1-12, wherein: the one or more kinematic chains include one or more robotic arms that are in an undocked state relative to the table top; and the one or more preset conditions permit spatial relationships between the table top and respective distal portions of the one or more robotic arms to change by more than a threshold amount during the first movement of the table top.

Clause 14. The patient platform system of any of Clauses 1-13, wherein the one or more kinematic chains include one or more robotic arms that are in an undocked state relative to the table top, and the one or more undocked robotic arms remain in non-interfering configurations during the first movement of the table top.

Clause 15. A method of operating a patient platform system that includes a table with a rigid base and a table top, the method comprising: receiving user request to move the table top relative to the rigid base; initiating first movement of the table top relative to the rigid base in accordance with the user request; and moving one or more kinematic chains relative to the rigid base in coordination with the first movement of the table top such that one or more preset conditions are maintained during the first movement of the table top, the one or more kinematic chains being coupled to the table.

Clause 16. The method of Clause 15, wherein the one or more kinematic chains include at least a first robotic arm.

Clause 17. The method of Clause 15 or 16, wherein the one or more preset conditions that are maintained during the first movement of the table top include a preset condition that limits movement of a first distal portion of a first kinematic chain to less than a threshold amount of movement relative to the table top.

Clause 18. The method of Clause 17, wherein the preset condition that limits movement of the first distal portion of the first kinematic chain to less than the threshold amount of movement relative to the table top comprises maintaining a remote center of motion associated with the first kinematic chain relative to the table top.

Clause 19. The method of any of Clauses 15-18, wherein the one or more preset conditions that are maintained during the first movement of the table top include a preset condition that prohibits one or more of: the one or more kinematic chains and the table top from reaching within a threshold distance of one another during the first movement of the table top; or kinematic chains of the one or more kinematic chains from reaching within a threshold distance of one another during the first movement of the table top.

Clause 20. The method of any of Clauses 15-19, wherein the one or more preset conditions that are maintained during the first movement of the table top include a preset condition that prevents the one or more kinematic chains from reaching within a threshold distance of one or more objects adjacent to the patient platform system.

Clause 21. The method of any of Clauses 15-20, wherein the one or more kinematic chains include a first kinematic chain that includes a first joint, wherein the one or more preset conditions that are maintained during the first movement of the table top include a preset condition that prevents the first joint from reaching beyond a joint limit.

Clause 22. The method of any of Clauses 15-21, wherein the one or more kinematic chains include at least a first robotic arm that has an attached medical tool at its distal end during the first movement of the table top.

Clause 23. The method of Clause 22, wherein the one or more preset conditions that are maintained during the first movement of the table top include a preset condition that maintains a fixed spatial relationship between the attached medical tool and the table top during the first movement of the table top.

Clause 24. The method of Clause 23, wherein the one or more preset conditions maintain an aligned spatial relationship to a tele-operation input device of the attached medical tool relative to the table top.

Clause 25. The method of any of Clauses 15-24, wherein the one or more kinematic chains include a first robotic arm and an adjustable arm support on which the first robotic arm is positioned.

Clause 26. The method of Clause 25, wherein moving the one or more kinematic chains relative to the rigid base in coordination with the first movement of the table top such that one or more preset conditions are maintained during the first movement of the table top includes moving the adjustable arm support and the first robotic arm in coordination with the first movement of the table top.

Clause 27. The method of any of Clauses 15-26, wherein: the one or more kinematic chains include one or more robotic arms that are in an undocked state relative to the table top; and the one or more preset conditions permit spatial relationships between the table top and respective distal portions of the one or more robotic arms to change by more than a threshold amount during the first movement of the table top.

Clause 28. The method of any of Clauses 15-27, wherein: the one or more kinematic chains include one or more robotic arms that are in an undocked state relative to the table top; and the one or more undocked robotic arms remain in non-interfering configurations during the first movement of the table top.

Clause 29. A non-transitory computer readable storage medium storing one or more programs configured for execution by one or more processors, the one or more programs comprising instructions for: receiving a user request to move a table top of a patient platform system, wherein: the patient platform system includes a table with the table top and a rigid base; and the table top is movable relative to a rigid base; initiating first movement of the table top relative to a rigid base in accordance with the user request; and moving one or more kinematic chains relative to the rigid base in coordination with the first movement of the table top such that one or more preset conditions are maintained during the first movement of the table top, wherein the one or more kinematic chains are coupled to the table.

Clause 30. The computer readable storage medium of Clause 29, wherein the one or more kinematic chains include at least a first robotic arm.

Clause 31. The computer readable storage medium of Clause 29 or 30, wherein the one or more preset conditions that are maintained during the first movement of the table top include a first preset condition that limits movement of a first distal portion of a first kinematic chain to less than a threshold amount of movement relative to the table top.

Clause 32. The computer readable storage medium of Clause 31, wherein the preset condition that limits movement of the first distal portion of the first kinematic chain to less than the threshold amount of movement relative to the table top comprises maintaining a remote center of motion associated with the first kinematic chain relative to the table top.

Clause 33. The computer readable storage medium of any of Clauses 29-32, wherein the one or more preset conditions that are maintained during the first movement of the table top include a preset condition that prohibits one or more of: the one or more kinematic chains and the table top from reaching within a threshold distance of one another during the first movement of the table top; or kinematic chains of the one or more kinematic chains from reaching within a threshold distance of one another during the first movement of the table top.

Clause 34. The computer readable storage medium of any of Clauses 29-33, wherein the one or more preset conditions that are maintained during the first movement of the table top include a preset condition that prevents the one or more kinematic chains from reaching within a threshold distance of one or more objects adjacent to the patient platform system.

Clause 35. The computer readable storage medium of any of Clauses 29-34, wherein the one or more kinematic chains include a first kinematic chain that includes a first joint, wherein the one or more preset conditions that are maintained during the first movement of the table top include a preset condition that prevents the first joint from reaching beyond a joint limit.

Clause 36. The computer readable storage medium of any of Clauses 29-35, wherein the one or more kinematic chains include at least a first robotic arm that has an attached medical tool at its distal end during the first movement of the table top.

Clause 37. The computer readable storage medium of Clause 36, wherein the one or more preset conditions that are maintained during the first movement of the table top include a preset condition that maintains a fixed spatial relationship between the attached medical tool and the table top during the first movement of the table top.

Clause 38. The computer readable storage medium of Clause 37, wherein the one or more preset conditions maintain an aligned spatial relationship to a tele-operation input device of the attached medical tool relative to the table top.

Clause 39. The computer readable storage medium of any of Clauses 29-38, wherein the one or more kinematic chains include a first robotic arm and an adjustable arm support on which the first robotic arm is positioned.

Clause 40. The computer readable storage medium of Clause 39, wherein the one or more programs further include instructions for moving the adjustable arm support and the first robotic arm in coordination with the first movement of the table top such that the one or more preset conditions are maintained during the first movement of the table top.

Clause 41. The computer readable storage medium of any of Clauses 29-40, wherein: the one or more kinematic chains include one or more robotic arms that are in an undocked state relative to the table top; and the one or more preset conditions permit spatial relationships between the table top and respective distal portions of the one or more robotic arms to change by more than a threshold amount during the first movement of the table top.

Clause 42. The computer readable storage medium of any of Clauses 29-41, wherein the one or more kinematic chains include one or more robotic arms that are in an undocked state relative to the table top, and the one or more undocked robotic arms remain in non-interfering configurations during the first movement of the table top.

Clause 43. A patient platform system, comprising: a table with a rigid base and a table top that is movable relative to the rigid base; a first robotic arm that is coupled to the table; one or more processors; and memory storing instructions, which, when executed by the one or more processors, cause the one or more processors to: initiate first movement of the table top relative to the rigid base; and constrain a change in spatial relationship between a first distal portion of the first robotic arm and the table top during the first movement of the table top.

Clause 44. The patient platform system of Clause 43, wherein constraining the change in spatial relationship between the first distal portion of the first robotic arm and the table top during the first movement of the table top includes in accordance with the first movement of the table top, moving at least a portion of the first robotic arm relative to the table top in a manner that limits movement of the first distal portion of the first robotic arm to less than a threshold amount of movement relative to the table top.

Clause 45. The patient platform system of Clause 44, wherein constraining the change in spatial relationship between the first distal portion of the first robotic arm and the table top during the first movement of the table top includes maintaining a remote center of motion associated with the first robotic arm relative to the table top.

Clause 46. The patient platform system of any of Clauses 43-45, further comprising an adjustable arm support, wherein: the first robotic arm is movably coupled to the adjustable arm support; and constraining the change in spatial relationship between a first distal end of the first robotic arm and the table top during the first movement of the table top includes: in accordance with the first movement of the table top, moving the adjustable arm support relative to the table top in a manner that limits movement of the first distal portion of the first robotic arm to less than a threshold amount of movement relative to the table top.

Clause 47. The patient platform system of any of Clauses 43-46, further comprising an adjustable arm support, wherein: the first robotic arm is movably coupled to the adjustable arm support; and constraining the change in spatial relationship between a first distal end of the first robotic arm and the table top during the first movement of the table top includes: in accordance with the first movement of the table top, coordinating movement of the first robotic arm relative to the adjustable arm support and movement of the adjustable arm support relative to the table top in a manner that limits movement of the first distal portion of the first robotic arm to less than a threshold amount of movement relative to the table top.

Clause 48. The patient platform system of any of Clauses 43-47, wherein the first robotic arm includes at least one kinematically redundant joint that is configured to move while the change in spatial relationship between the first distal portion of the first robotic arm and the table top is constrained.

Clause 49. The patient platform system of any of Clauses 43-48, further comprising a second robotic arm in addition to the first robotic arm, wherein the stored instructions, when executed by the one or more processors, cause the one or more processors to perform any of: (a) moving any of the first robotic arm and the second robotic arm in a coordinated manner such that collision between the first robotic arm and the second robotic arm is avoided during the first movement of the table top; (b) moving any of the first robotic arm and the second robotic arm to avoid self-collision; (c) moving any of the first robotic arm and the second robotic arm for joint limit avoidance; and (d) moving any of the first robotic arm and the second robotic arm to avoid collision with any of the table and one or more structures that are operable coupled to the table.

Clause 50. The patient platform system of Clause 49, further comprising an adjustable arm support configured to support at least one of the first robotic arm or the second robotic arm, wherein the stored instructions, when executed by the one or more processors, cause the one or more processors to perform any of: (e) moving the adjustable arm support and at least one of the first robotic arm or the second robotic arm to avoid collision with any of the table and one or more structures that are operable coupled to the table; and (f) moving the adjustable arm support and at least one of the first robotic arm or the second robotic arm to avoid collision with a ground that supports the table.

Clause 51. A method of operating a patient platform system that includes a table with a rigid base and a table top, the method comprising: initiating first movement of a table top relative to a rigid base; and constraining a change in spatial relationship between a first distal portion of a first robotic arm and the table top during the first movement of the table top, wherein the first robotic arm is coupled to the table.

Clause 52. The method of Clause 51, wherein constraining the change in spatial relationship between the first distal portion of the first robotic arm and the table top during the first movement of the table top includes in accordance with the first movement of the table top, moving at least a portion of the first robotic arm relative to the table top in a manner that limits movement of the first distal portion of the first robotic arm to less than a threshold amount of movement relative to the table top.

Clause 53. The method of Clause 52, wherein constraining the change in spatial relationship between the first distal portion of the first robotic arm and the table top during the first movement of the table top includes maintaining a remote center of motion associated with the first robotic arm relative to the table top.

Clause 54. The method of any of Clauses 51-53, wherein: the patient platform system further includes an adjustable arm support; the first robotic arm is movably coupled to the adjustable arm support; and constraining the change in spatial relationship between a first distal end of the first robotic arm and the table top during the first movement of the table top includes: in accordance with the first movement of the table top, moving the adjustable arm support relative to the table top in a manner that limits movement of the first distal portion of the first robotic arm to less than a threshold amount of movement relative to the table top.

Clause 55. The method of any of Clauses 51-54, wherein: the patient platform system further includes an adjustable arm support; the first robotic arm is movably coupled to the adjustable arm support; and constraining the change in spatial relationship between a first distal end of the first robotic arm and the table top during the first movement of the table top includes: in accordance with the first movement of the table top, coordinating movement of the first robotic arm relative to the adjustable arm support and movement of the adjustable arm support relative to the table top in a manner that limits movement of the first distal portion of the first robotic arm to less than a threshold amount of movement relative to the table top.

Clause 56. The method of any of Clauses 51-55, wherein the first robotic arm includes at least one kinematically redundant joint that is configured to move while the change in spatial relationship between the first distal portion of the first robotic arm and the table top is constrained.

Clause 57. The method of any of Clauses 51-56, wherein the one or more kinematic chains further include a second robotic arm in addition to the first robotic arm, the method further comprising any of: (a) moving any of the first robotic arm and the second robotic arm in a coordinated manner such that collision between the first robotic arm and the second robotic arm is avoided during the first movement of the table top; (b) moving any of the first robotic arm and the second robotic arm to avoid self-collision; (c) moving any of the first robotic arm and the second robotic arm for joint limit avoidance; and (d) moving any of the first robotic arm and the second robotic arm to avoid collision with any of the table and one or more structures that are operable coupled to the table.

Clause 58. The method of Clause 57, further comprising an adjustable arm configured to support at least one of the first robotic arm or the second robotic arm, the method further comprising any of: (e) moving the adjustable arm support and at least one of the first robotic arm or the second robotic arm to avoid collision with any of the table and one or more structures that are operable coupled to the table; and (f) moving the adjustable arm support and at least one of the first robotic arm or the second robotic arm to avoid collision with a ground that supports the table.

Clause 59. A non-transitory computer readable storage medium storing one or more programs configured for execution by a computer system having one or more processors, memory, and a display, the one or more programs comprising instructions for: initiating first movement of a table top of a patient platform system, wherein: the patient platform system includes a table with a rigid base and the table top; and the table top is movable relative to the rigid base; and constraining a change in spatial relationship between a first distal portion of a first robotic arm and the table top during the first movement of the table top, the first robotic arm being coupled to the table.

Clause 60. The computer readable storage medium of Clause 59, wherein constraining the change in spatial relationship between the first distal portion of the first robotic arm and the table top during the first movement of the table top includes in accordance with the first movement of the table top, moving at least a portion of the first robotic arm relative to the table top in a manner that limits movement of the first distal portion of the first robotic arm to less than a threshold amount of movement relative to the table top.

Clause 61. The computer readable storage medium of Clause 60, wherein constraining the change in spatial relationship between the first distal portion of the first robotic arm and the table top during the first movement of the table top includes maintaining a remote center of motion associated with the first robotic arm relative to the table top.

Clause 62. The computer readable storage medium of any of Clauses 59-61, wherein: the patient platform system further includes an adjustable arm support; the first robotic arm is movably coupled to the adjustable arm support; and constraining the change in spatial relationship between a first distal end of the first robotic arm and the table top during the first movement of the table top includes: in accordance with the first movement of the table top, moving the adjustable arm support relative to the table top in a manner that limits movement of the first distal portion of the first robotic arm to less than a threshold amount of movement relative to the table top.

Clause 63. The computer readable storage medium of any of Clauses 59-62, wherein: the patient platform system further includes an adjustable arm support; the first robotic arm is movably coupled to the adjustable arm support; and constraining the change in spatial relationship between a first distal end of the first robotic arm and the table top during the first movement of the table top includes: in accordance with the first movement of the table top, coordinating movement of the first robotic arm relative to the adjustable arm support and movement of the adjustable arm support relative to the table top in a manner that limits movement of the first distal portion of the first robotic arm to less than a threshold amount of movement relative to the table top.

Clause 64. The computer readable storage medium of any of Clauses 59-63, wherein the first robotic arm includes at least one kinematically redundant joint that is configured to move while the change in spatial relationship between the first distal portion of the first robotic arm and the table top is constrained.

Clause 65. The computer readable storage medium of any of Clauses 59-64, wherein the patient platform system further includes a second robotic arm in addition to the first robotic arm, the one or more programs further include instructions for any of: (a) moving any of the first robotic arm and the second robotic arm in a coordinated manner such that collision between the first robotic arm and the second robotic arm is avoided during the first movement of the table top; (b) moving any of the first robotic arm and the second robotic arm to avoid self-collision; (c) moving any of the first robotic arm and the second robotic arm for joint limit avoidance; and (d) move any of the first robotic arm and the second robotic arm to avoid collision with any of the table and one or more structures that are operable coupled to the table.

Clause 66. The computer readable storage medium of Clause 65, wherein the patient platform system further includes an adjustable arm support configured to support at least one of the first robotic arm or the second robotic arm, the one or more programs further include instructions for any of: (e) move the adjustable arm support and at least one of the first robotic arm or the second robotic arm to avoid collision with any of the table and one or more structures that are operable coupled to the table; and (f) move the adjustable arm support and at least one of the first robotic arm or the second robotic arm to avoid collision with a ground that supports the table.

Clause 67. The computer readable storage medium of any of Clauses 59-66, wherein: the one or more robotic arms further include a second robotic arm in addition to the first robotic arm; and the one or more programs further include instructions for moving the second robotic arm and the first robotic arm in a coordinated manner such that collision between the first robotic arm and the second robotic arm is avoided during the first movement of the table top.

Clause 68. A patient platform system, comprising: a table with a rigid base and a table top that is movable relative to the rigid base; a first robotic arm; one or more sensors that are positioned to detect one or more forces exerted on the first robotic arm; one or more processors; and memory storing instructions, which, when executed by the one or more processors, cause the one or more processors to: initiate first movement of the table top relative to the rigid base; move the first robotic arm in coordination with the first movement of the table top; and obtain, from the one or more sensors, sensor information regarding one or more forces exerted on the first robotic arm during the first movement of the table top and movement of the first robotic arm in coordination with the first movement of the table top.

Clause 69. The patient platform system of Clause 68, wherein the one or more forces exerted on the first robotic arm include a force component associated with gravity of a patient positioned on the table top.

Clause 70. The patient platform system of Clause 68 or 69, wherein the stored instructions, when executed by the one or more processors, cause the one or more processors to in accordance with the sensor information, constrain a change in spatial relationship between a first distal portion of the first robotic arm and the table top during the first movement of the table top.

Clause 71. The patient platform system of Clause 70, wherein constraining the change in spatial relationship between the first distal portion of the first robotic arm and the table top during the first movement of the table top includes: in accordance with a determination that the sensor information meets first criteria, constraining movement of the first distal portion of the first robotic arm relative to the table top based on a first constraint; and in accordance with a determination that the sensor information does not meet the first criteria, constraining movement of the first distal portion of the first robotic arm relative to the table top based on a second constraint that is different from the first constraint.

Clause 72. The patient platform system of Clause 70 or 71, wherein the first robotic arm includes an attached surgical tool that is retracted from the first distal portion of the first robotic arm away from the table top.

Clause 73. The patient platform system of any of Clauses 68-72, wherein the one or more forces exerted on the first robotic arm include a force component associated with impact from an object external to the patient platform system.

Clause 74. The patient platform system of Clause 73, wherein the stored instructions further include instructions, which, when executed by the one or more processors, cause the one or more processors to in accordance with the sensor information, activate power-assisted movement of the first robotic arm during the first movement of the table top.

Clause 75. A method of operating a patient platform system that includes a first robotic arm, a table with a rigid base and a table top, and one or more sensors positioned to detect one or more forces exerted on the first robotic arm, the method comprising: initiating first movement of the table top relative to the rigid base; moving the first robotic arm in coordination with the first movement of the table top; and obtaining, from the one or more sensors, sensor information regarding one or more forces exerted on the first robotic arm during the first movement of the table top and movement of the first robotic arm in coordination with the first movement of the table top.

Clause 76. The method of Clause 75, wherein the one or more forces exerted on the first robotic arm include a force component associated with gravity of a patient positioned on the table top.

Clause 77. The method of Clause 75 or 76, further comprising in accordance with the sensor information, constraining a change in spatial relationship between a first distal portion of the first robotic arm and the table top during the first movement of the table top.

Clause 78. The method of Clause 77, wherein constraining the change in spatial relationship between the first distal portion of the first robotic arm and the table top during the first movement of the table top includes: in accordance with a determination that the sensor information meets first criteria, constraining movement of the first distal portion of the first robotic arm relative to the table top based on a first constraint; and in accordance with a determination that the sensor information does not meet the first criteria, constraining movement of the first distal portion of the first robotic arm relative to the table top based on a second constraint that is different from the first constraint.

Clause 79. The method of Clause 77 or 78, wherein the first robotic arm includes an attached surgical tool that is retracted from the first distal portion of the first robotic arm away from the table top.

Clause 80. The method of any of Clauses 75-79, wherein the one or more forces exerted on the first robotic arm include a force component associated with impact from an object external to the patient platform system.

Clause 81. The method of Clause 80, further comprising in accordance with the sensor information, activating power-assisted movement of the first robotic arm during the first movement of the table top.

Clause 82. A non-transitory computer readable storage medium storing one or more programs configured for execution by one or more processors, the one or more programs comprising instructions for: initiating first movement of a table top of a patient platform system, wherein: the patient platform system includes a first robotic arm, a table with a rigid base and the table top, and one or more sensors that are positioned to detect one or more forces exerted on the first robotic arm; and the table top is movable relative to the rigid base; moving the first robotic arm in coordination with the first movement of the table top; and obtaining, from one or more sensors, sensor information regarding one or more forces exerted on the first robotic arm during the first movement of the table top and movement of the first robotic arm in coordination with the first movement of the table top.

Clause 83. The computer readable storage medium of Clause 82, wherein the one or more forces exerted on the first robotic arm include a force component associated with gravity of a patient positioned on the table top.

Clause 84. The computer readable storage medium of Clause 82 or 83, wherein the one or more programs further include instructions for in accordance with the sensor information, constraining a change in spatial relationship between a first distal portion of the first robotic arm and the table top during the first movement of the table top.

Clause 85. The computer readable storage medium of Clause 84, wherein constraining the change in spatial relationship between the first distal portion of the first robotic arm and the table top during the first movement of the table top includes: in accordance with a determination that the sensor information meets first criteria, constraining movement of the first distal portion of the first robotic arm relative to the table top based on a first constraint; and in accordance with a determination that the sensor information does not meet the first criteria, constraining movement of the first distal portion of the first robotic arm relative to the table top based on a second constraint that is different from the first constraint.

Clause 86. The computer readable storage medium of Clause 84 or 85, wherein the first robotic arm includes an attached surgical tool that is retracted from the first distal portion of the first robotic arm away from the table top.

Clause 87. The computer readable storage medium of any of Clauses 82-86, wherein the one or more forces exerted on the first robotic arm include a force component associated with impact from an object external to the patient platform system.

Clause 88. The computer readable storage medium of Clause 87, wherein the one or more programs further comprising instructions for in accordance with the sensor information, activating power-assisted movement of the first robotic arm during the first movement of the table top.

Clause 89. A patient platform system, comprising: a table with a rigid base and a table top that is movable relative to the rigid base; a first robotic arm; one or more processors; and memory storing instructions, which, when executed by the one or more processors, cause the one or more processors to: move the first robotic arm in coordination with first movement of the table top relative to the rigid base; and halt at least one of movement of first robotic arm or the first movement of the table top in accordance with a determination that one or more criteria are met.

Clause 90. The patient platform system of Clause 89, wherein halting the at least one of the movement of first robotic arm or the first movement of the table top in accordance with the determination that the one or more criteria are met includes halting the at least one of the movement of first robotic arm or the first movement of the table top in accordance with receipt of a user input corresponding to a request to halt the at least one of the movement of the first robotic arm or the first movement of the table top.

Clause 91. The patient platform system of Clause 89 or 90, wherein halting the at least one of the movement of first robotic arm or the first movement of the table top in accordance with the determination that the one or more criteria are met includes halting the at least one of the movement of first robotic arm or the first movement of the table top in accordance with detection of a collision with the first robotic arm or the table top or anticipation of a collision with the first robotic arm or the table top that are not resolvable with permitted movement of the first robotic arm or the table top.

Clause 92. The patient platform system of any of Clauses 89-91, wherein halting the at least one of the movement of first robotic arm or the first movement of the table top in accordance with a determination that the one or more criteria are met includes halting the at least one of the movement of first robotic arm or the first movement of the table top in accordance with determining that at least one of the first robotic arm or the table top has reached an associated joint limit.

Clause 93. The patient platform system of any of Clauses 89-92, wherein halting the at least one of the movement of first robotic arm or the first movement of the table top in accordance with a determination that the one or more criteria are met includes halting the at least one of the movement of first robotic arm or the first movement of the table top in accordance with detecting that a force exerted on the first robotic arm has exceeded a preset force threshold.

Clause 94. The patient platform system of any of Clauses 89-93, further comprising a display, wherein the stored instructions further include instructions, which, when executed by the one or more processors, cause the one or more processors to: after halting the at least one of movement of the first robotic arm or the first movement of the table top, in accordance with the determination that one or more criteria are met, performing one or more of: presenting, at the display, information regarding the one or more criteria that are met; or presenting, at the display, a graphical user interface for user intervention of at least one of: movement of the first robotic arm or movement of the table top.

Clause 95. A method of operating a patient platform system that includes a first robotic arm and a table with a rigid base and a table top, the method comprising: moving the first robotic arm in coordination with first movement of the table top relative to the rigid base; and halting at least one of movement of first robotic arm or the first movement of the table top in accordance with a determination that one or more criteria are met.

Clause 96. The method of Clause 95, wherein halting the at least one of the movement of first robotic arm or the first movement of the table top in accordance with the determination that the one or more criteria are met includes halting the at least one of the movement of first robotic arm or the first movement of the table top in accordance with receipt of a user input corresponding to a request to halt the at least one of the movement of the first robotic arm or the first movement of the table top.

Clause 97. The method of Clause 95 or 96, wherein halting the at least one of the movement of first robotic arm or the first movement of the table top in accordance with the determination that the one or more criteria are met includes halting the at least one of the movement of first robotic arm or the first movement of the table top in accordance with detection of a collision with the first robotic arm or the table top or anticipation of a collision with the first robotic arm or the table top that are not resolvable with permitted movement of the first robotic arm or the table top.

Clause 98. The method of any of Clauses 95-97, wherein halting the at least one of the movement of first robotic arm or the first movement of the table top in accordance with a determination that the one or more criteria are met includes halting the at least one of the movement of first robotic arm or the first movement of the table top in accordance with determining that at least one of the first robotic arm or the table top has reached an associated joint limit.

Clause 99. The method of any of Clauses 95-98, wherein halting the at least one of the movement of first robotic arm or the first movement of the table top in accordance with a determination that the one or more criteria are met includes halting the at least one of the movement of first robotic arm or the first movement of the table top in accordance with detecting that a force exerted on the first robotic arm has exceeded a preset force threshold.

Clause 100. The method of any of Clauses 95-99, wherein the patient platform system further comprises a display, the method further comprising, after halting the at least one of movement of the first robotic arm or the first movement of the table top, in accordance with the determination that one or more criteria are met, performing one or more of: presenting, at the display, information regarding the one or more criteria that are met; or presenting, at the display, a graphical user interface for user intervention of at least one of: movement of the first robotic arm or movement of the table top.

Clause 101. A non-transitory computer readable storage medium storing one or more programs configured for execution by one or more processors, the one or more programs comprising instructions for: moving a first robotic arm in coordination with first movement of a table top relative to a rigid base; and halting at least one of movement of first robotic arm or the first movement of the table top in accordance with a determination that one or more criteria are met.

Clause 102. The computer readable storage medium of Clause 101, wherein halting the at least one of the movement of first robotic arm or the first movement of the table top in accordance with the determination that the one or more criteria are met includes halting the at least one of the movement of first robotic arm or the first movement of the table top in accordance with receipt of a user input corresponding to a request to halt the at least one of the movement of the first robotic arm or the first movement of the table top.

Clause 103. The computer readable storage medium of Clause 101 or 102, wherein halting the at least one of the movement of first robotic arm or the first movement of the table top in accordance with the determination that the one or more criteria are met includes halting the at least one of the movement of first robotic arm or the first movement of the table top in accordance with detection of a collision with the first robotic arm or the table top or anticipation of a collision with the first robotic arm or the table top that are not resolvable with permitted movement of the first robotic arm or the table top.

Clause 104. The computer readable storage medium of any of Clauses 101-103, wherein halting the at least one of the movement of first robotic arm or the first movement of the table top in accordance with a determination that the one or more criteria are met includes halting the at least one of the movement of first robotic arm or the first movement of the table top in accordance with determining that at least one of the first robotic arm or the table top has reached an associated joint limit.

Clause 105. The computer readable storage medium of any of Clauses 101-104, wherein halting the at least one of the movement of first robotic arm or the first movement of the table top in accordance with a determination that the one or more criteria are met includes halting the at least one of the movement of first robotic arm or the first movement of the table top in accordance with detecting that a force exerted on the first robotic arm has exceeded a preset force threshold.

Clause 106. The computer readable storage medium of any of Clauses 101-105, wherein the one or more programs further comprise instructions for: after halting the at least one of movement of the first robotic arm or the first movement of the table top, in accordance with the determination that one or more criteria are met, performing one or more of: presenting, at a display, information regarding the one or more criteria that are met; or presenting, at the display, a graphical user interface for user intervention of at least one of: movement of the first robotic arm or movement of the table top.

1. A patient platform system comprising:
a table with a rigid base and a table top that is movable relative to the rigid base;
one or more kinematic chains that are coupled to the table;
one or more processors; and
memory storing instructions, which, when executed by the one or more processors, cause the one or more processors to:
initiate first movement of the table top relative to the rigid base in accordance with a user request; and
move the one or more kinematic chains relative to the rigid base in coordination with the first movement of the table top such that one or more preset conditions are maintained during the first movement of the table top.

2. The patient platform system of claim 1, wherein the one or more kinematic chains include at least a first robotic arm.

3. The patient platform system of claim 1, wherein the one or more preset conditions that are maintained during the first movement of the table top include a preset condition that limits movement of a first distal portion of a first kinematic chain to less than a threshold amount of movement relative to the table top.

4. The patient platform system of claim 3, wherein the preset condition that limits the movement of the first distal portion of the first kinematic chain to less than the threshold amount of movement relative to the table top comprises maintaining a remote center of motion associated with the first kinematic chain relative to the table top.

5. The patient platform system of claim 1, wherein the one or more preset conditions that are maintained during the first movement of the table top include a preset condition that prohibits one or more of:
the one or more kinematic chains and the table top from reaching within a threshold distance of one another during the first movement of the table top; or
kinematic chains of the one or more kinematic chains from reaching within a threshold distance of one another during the first movement of the table top.

6. The patient platform system of claim 1, wherein the one or more preset conditions that are maintained during the first movement of the table top include a preset condition that prevents the one or more kinematic chains from reaching within a threshold distance of one or more objects adjacent to the patient platform system.

7. The patient platform system of claim 1, wherein the one or more kinematic chains include a first kinematic chain that includes a first joint, wherein the one or more preset conditions that are maintained during the first movement of the table top include a preset condition that prevents the first joint from reaching beyond a joint limit.

8. The patient platform system of claim 1, wherein the one or more kinematic chains include at least a first robotic arm that has an attached medical tool at its distal end during the first movement of the table top.

9. The patient platform system of claim 8, wherein the one or more preset conditions that are maintained during the first movement of the table top include a preset condition that maintains a fixed spatial relationship between the attached medical tool and the table top during the first movement of the table top.

10. The patient platform system of claim 8, wherein the one or more preset conditions maintain an aligned spatial relationship to a tele-operation input device of the attached medical tool relative to the table top.

11. The patient platform system of claim 1, wherein the one or more kinematic chains include a first robotic arm and an adjustable arm support on which the first robotic arm is positioned.

12. The patient platform system of claim 11, wherein the instructions, when executed by the one or more processors, cause the one or more processors to move the adjustable arm support and the first robotic arm in coordination with the first movement of the table top such that the one or more preset conditions are maintained during the first movement of the table top.

13. The patient platform system of claim 1, wherein:
the one or more kinematic chains include one or more robotic arms that are in an undocked state relative to the table top; and
the one or more preset conditions permit spatial relationships between the table top and respective distal portions of the one or more robotic arms to change by more than a threshold amount during the first movement of the table top.

14. The patient platform system of claim 1, wherein the one or more kinematic chains include one or more robotic arms that are in an undocked state relative to the table top, and the one or more undocked robotic arms remain in non-interfering configurations during the first movement of the table top.

15. A method of operating a patient platform system that includes a table with a rigid base and a table top, the method comprising:
receiving user request to move the table top relative to the rigid base;
initiating first movement of the table top relative to the rigid base in accordance with the user request; and
moving one or more kinematic chains relative to the rigid base in coordination with the first movement of the table top such that one or more preset conditions are maintained during the first movement of the table top, the one or more kinematic chains being coupled to the table.

16. The method of claim 15, wherein the one or more kinematic chains include at least a first robotic arm.

17. The method of claim 15, wherein the one or more preset conditions that are maintained during the first movement of the table top include a preset condition that limits movement of a first distal portion of a first kinematic chain to less than a threshold amount of movement relative to the table top.

18. The method of claim 17, wherein the preset condition that limits movement of the first distal portion of the first kinematic chain to less than the threshold amount of movement relative to the table top comprises maintaining a remote center of motion associated with the first kinematic chain relative to the table top.

19. The method of claim 15, wherein the one or more preset conditions that are maintained during the first movement of the table top include a preset condition that prohibits one or more of:
the one or more kinematic chains and the table top from reaching within a threshold distance of one another during the first movement of the table top; or
kinematic chains of the one or more kinematic chains from reaching within a threshold distance of one another during the first movement of the table top.

20. The method of claim 15, wherein the one or more preset conditions that are maintained during the first movement of the table top include a preset condition that prevents the one or more kinematic chains from reaching within a threshold distance of one or more objects adjacent to the patient platform system.

21. The method of claim 15, wherein the one or more kinematic chains include a first kinematic chain that includes a first joint, wherein the one or more preset conditions that are maintained during the first movement of the table top include a preset condition that prevents the first joint from reaching beyond a joint limit.

22. The method of claim 15, wherein the one or more kinematic chains include at least a first robotic arm that has an attached medical tool at its distal end during the first movement of the table top.

23. The method of claim 22, wherein the one or more preset conditions that are maintained during the first movement of the table top include a preset condition that maintains a fixed spatial relationship between the attached medical tool and the table top during the first movement of the table top.

24. The method of claim 23, wherein the one or more preset conditions maintain an aligned spatial relationship to a tele-operation input device of the attached medical tool relative to the table top.

25. The method of claim 15, wherein the one or more kinematic chains include a first robotic arm and an adjustable arm support on which the first robotic arm is positioned.

26. The method of claim 25, wherein moving the one or more kinematic chains relative to the rigid base in coordination with the first movement of the table top such that one or more preset conditions are maintained during the first movement of the table top includes moving the adjustable arm support and the first robotic arm in coordination with the first movement of the table top.

27. The method of claim 15, wherein:
the one or more kinematic chains include one or more robotic arms that are in an undocked state relative to the table top; and
the one or more preset conditions permit spatial relationships between the table top and respective distal portions of the one or more robotic arms to change by more than a threshold amount during the first movement of the table top.

28. The method of claim 15, wherein:
the one or more kinematic chains include one or more robotic arms that are in an undocked state relative to the table top; and
the one or more undocked robotic arms remain in non-interfering configurations during the first movement of the table top.

29. A non-transitory computer readable storage medium storing one or more programs configured for execution by one or more processors, the one or more programs comprising instructions for:
receiving a user request to move a table top of a patient platform system, wherein:
the patient platform system includes a table with the table top and a rigid base; and
the table top is movable relative to a rigid base;
initiating first movement of the table top relative to a rigid base in accordance with the user request; and
moving one or more kinematic chains relative to the rigid base in coordination with the first movement of the table top such that one or more preset conditions are maintained during the first movement of the table top, wherein the one or more kinematic chains are coupled to the table.

30. The computer readable storage medium of claim 29, wherein the one or more kinematic chains include at least a first robotic arm.

31. The computer readable storage medium of claim 29, wherein the one or more preset conditions that are maintained during the first movement of the table top include a first preset condition that limits movement of a first distal portion of a first kinematic chain to less than a threshold amount of movement relative to the table top.

32. The computer readable storage medium of claim 31, wherein the preset condition that limits movement of the first distal portion of the first kinematic chain to less than the threshold amount of movement relative to the table top comprises maintaining a remote center of motion associated with the first kinematic chain relative to the table top.

33. The computer readable storage medium of claim 29, wherein the one or more preset conditions that are maintained during the first movement of the table top include a preset condition that prohibits one or more of:
the one or more kinematic chains and the table top from reaching within a threshold distance of one another during the first movement of the table top; or
kinematic chains of the one or more kinematic chains from reaching within a threshold distance of one another during the first movement of the table top.

34. The computer readable storage medium of claim 29, wherein the one or more preset conditions that are maintained during the first movement of the table top include a preset condition that prevents the one or more kinematic chains from reaching within a threshold distance of one or more objects adjacent to the patient platform system.

35. The computer readable storage medium of claim 29, wherein the one or more kinematic chains include a first kinematic chain that includes a first joint, wherein the one or more preset conditions that are maintained during the first movement of the table top include a preset condition that prevents the first joint from reaching beyond a joint limit.

36. The computer readable storage medium of claim 29, wherein the one or more kinematic chains include at least a first robotic arm that has an attached medical tool at its distal end during the first movement of the table top.

37. The computer readable storage medium of claim 36, wherein the one or more preset conditions that are maintained during the first movement of the table top include a preset condition that maintains a fixed spatial relationship between the attached medical tool and the table top during the first movement of the table top.

38. The computer readable storage medium of claim 37, wherein the one or more preset conditions maintain an aligned spatial relationship to a tele-operation input device of the attached medical tool relative to the table top.

39. The computer readable storage medium of claim 29, wherein the one or more kinematic chains include a first robotic arm and an adjustable arm support on which the first robotic arm is positioned.

40. The computer readable storage medium of claim 39, wherein the one or more programs further include instructions for moving the adjustable arm support and the first robotic arm in coordination with the first movement of the table top such that the one or more preset conditions are maintained during the first movement of the table top.

41. The computer readable storage medium of claim 29, wherein:
the one or more kinematic chains include one or more robotic arms that are in an undocked state relative to the table top; and
the one or more preset conditions permit spatial relationships between the table top and respective distal portions of the one or more robotic arms to change by more than a threshold amount during the first movement of the table top.

42. The computer readable storage medium of claim 29, wherein the one or more kinematic chains include one or more robotic arms that are in an undocked state relative to the table top, and the one or more undocked robotic arms remain in non-interfering configurations during the first movement of the table top.

43.-106. (canceled)
